SEQUENCE LISTING (65 sequences)

SEQ ID NO: 1 - DNA, 30 bp, Artificial sequence
  primer
gacgacgacaagatgattttagatgtggat

SEQ ID NO: 2 - DNA, 30 bp, Artificial sequence
  primer
ggaacaagacccgtctaggattttttaatg

SEQ ID NO: 3 - DNA, 23 bp, Artificial sequence
  primer
gacgttgtaaaacgacggccagn

SEQ ID NO: 4 - DNA, 22 bp, Artificial sequence
  primer
acgttgtaaaacgacggccagt

SEQ ID NO: 5 - DNA, 31 bp, Artificial sequence
  primer
caatttcacacaggaaacagctatgaccatg

SEQ ID NO: 6 - DNA, 37 bp, Artificial sequence
  primer
gaacatccccaagatgaacccactattagagaaaaag

SEQ ID NO: 7 - DNA, 37 bp, Artificial sequence
  primer
ctttttctctaatagtgggttcatcttggggatgttc

SEQ ID NO: 8 - DNA, 37 bp, Artificial sequence
  primer
gaacatccccaagatagacccactattagagaaaaag

SEQ ID NO: 9 - DNA, 37 bp, Artificial sequence
  primer
ctttttctctaatagtgggtctatcttggggatgttc

SEQ ID NO: 10 - DNA, 37 bp, Artificial sequence
  primer
gaacatccccaagataaccccactattagagaaaaag

SEQ ID NO: 11 - DNA, 37 bp, Artificial sequence
  primer
ctttttctctaatagtggggttatcttggggatgttc

SEQ ID NO: 12 - DNA, 37 bp, Artificial sequence
  primer
gaacatccccaagatcaccccactattagagaaaaag

SEQ ID NO: 13 - DNA, 37 bp, Artificial sequence
  primer
ctttttctctaatagtggggtgatcttggggatgttc

SEQ ID NO: 14 - DNA, 37 bp, Artificial sequence
  primer
gaacatccccaagatnnkcccactattagagaaaaag

SEQ ID NO: 15 - DNA, 18 bp, Artificial sequence
  primer
gacagtcactccggcccg

SEQ ID NO: 16 - DNA, 18 bp, Artificial sequence
  primer
cgacgactcgtggagccc

SEQ ID NO: 17 - DNA, 2328 bp, Pyrococcus furiosus
  misc_feature (277)..(279): nnn= AGA, AGG, CGA, CGC, CGG, CGT (ALL POSSIBLE CODONS FOR ARGININE)
atgattttagatgtggattacataactgaagaaggaaaacctgttattaggctattcaaaaaagagaacggaaaatttaagatagagcatgatagaacttttagaccatacatttacgctcttctcagggatgattcaaagattgaagaagttaagaaaataacgggggaaaggcatggaaagattgtgagaattgttgatgtagagaaggttgagaaaaagtttctcggcaagcctattaccgtgtggaaactttatttggaacatccccaagatnnncccactattagagaaaaagttagagaacatccagcagttgtggacatcttcgaatacgatattccatttgcaaagagatacctcatcgacaaaggcctaataccaatggagggggaagaagagctaaagattcttgccttcgatatagaaaccctctatcacgaaggagaagagtttggaaaaggcccaattataatgattagttatgcagatgaaaatgaagcaaaggtgattacttggaaaaacatagatcttccatacgttgaggttgtatcaagcgagagagagatgataaagagatttctcaggattatcagggagaaggatcctgacattatagttacttataatggagactcattcgcattcccatatttagcgaaaagggcagaaaaacttgggattaaattaaccattggaagagatggaagcgagcccaagatgcagagaataggcgatatgacggctgtagaagtcaagggaagaatacatttcgacttgtatcatgtaataacaaggacaataaatctcccaacatacacactagaggctgtatatgaagcaatttttggaaagccaaaggagaaggtatacgccgacgagatagcaaaagcctgggaaagtggagagaaccttgagagagttgccaaatactcgatggaagatgcaaaggcaacttatgaactcgggaaagaattccttccaatggaaattcagctttcaagattagttggacaacctttatgggatgtttcaaggtcaagcacagggaaccttgtagagtggttcttacttaggaaagcctacgaaagaaacgaagtagctccaaacaagccaagtgaagaggagtatcaaagaaggctcagggagagctacacaggtggattcgttaaagagccagaaaaggggttgtgggaaaacatagtatacctagattttagagccctatatccctcgattataattacccacaatgtttctcccgatactctaaatcttgagggatgcaagaactatgatatcgctcctcaagtaggccacaagttctgcaaggacatccctggttttataccaagtctcttgggacatttgttagaggaaagacaaaagattaagacaaaaatgaaggaaactcaagatcctatagaaaaaatactccttgactatagacaaaaagcgataaaactcttagcaaattctttctacggatattatggctatgcaaaagcaagatggtactgtaaggagtgtgctgagagcgttactgcctggggaagaaagtacatcgagttagtatggaaggagctcgaagaaaagtttggatttaaagtcctctacattgacactgatggtctctatgcaactatcccaggaggagaaagtgaggaaataaagaaaaaggctctagaatttgtaaaatacataaattcaaagctccctggactgctagagcttgaatatgaagggttttataagaggggattcttcgttacgaagaagaggtatgcagtaatagatgaagaaggaaaagtcattactcgtggtttagagatagttaggagagattggagtgaaattgcaaaagaaactcaagctagagttttggagacaatactaaaacacggagatgttgaagaagctgtgagaatagtaaaagaagtaatacaaaagcttgccaattatgaaattccaccagagaagctcgcaatatatgagcagataacaagaccattacatgagtataaggcgataggtcctcacgtagctgttgcaaagaaactagctgctaaaggagttaaaataaagccaggaatggtaattggatacatagtacttagaggcgatggtccaattagcaatagggcaattctagctgaggaatacgatcccaaaaagcacaagtatgacgcagaatattacatggagaaccaggttcttccagcggtacttaggatattggagggatttggatacagaaaggaagacctcagataccaaaagacaagacaagtcggcctaacttcctggcttaacattaaaaaatcctag

SEQ ID NO: 18 - DNA, 2328 bp, Pyrococcus furiosus; identical to SEQ ID NO: 17
  misc_feature (277)..(279): nnn = GAA, GAG (ALL CODONS FOR GLUTAMIC ACID)

SEQ ID NO: 19 - DNA, 2328 bp, Pyrococcus furiosus
  misc_feature (277)..(279): nnn = AGA, AGG, CGA, CGC, CGG, CGT (ALL POSSIBLE CODONS FOR ARGININE)
atgattttagatgtggattacataactgaagaaggaaaacctgttattaggctattcaaaaaagagaacggaaaatttaagatagagcatgatagaacttttagaccatacatttacgctcttctcagggatgattcaaagattgaagaagttaagaaaataacgggggaaaggcatggaaagattgtgagaattgttgatgtagagaaggttgagaaaaagtttctcggcaagcctattaccgtgtggaaactttatttggaacatccccaagatnnncccactattagagaaaaagttagagaacatccagcagttgtggacatcttcgaatacgatattccatttgcaaagagatacctcatcgacaaaggcctaataccaatggagggggaagaagagctaaagattcttgccttcgatatagaaaccctctatcacgaaggagaagagtttggaaaaggcccaattataatgattagttatgcagatgaaaatgaagcaaaggtgattacttggaaaaacatagatcttccatacgttgaggttgtatcaagcgagagagagatgataaagagatttctcaggattatcagggagaaggatcctgacattatagttacttataatggagactcattcgcattcccatatttagcgaaaagggcagaaaaacttgggattaaattaaccattggaagagatggaagcgagcccaagatgcagagaataggcgatatgacggctgtagaagtcaagggaagaatacatttcgacttgtatcatgtaataacaaggacaataaatctcccaacatacacactagaggctgtatatgaagcaatttttggaaagccaaaggagaaggtatacgccgacgagatagcaaaagcctgggaaagtggagagaaccttgagagagttgccaaatactcgatggaagatgcaaaggcaacttatgaactcgggaaagaattccttccaatggaaattcagctttcaagattagttggacaacctttatgggatgtttcaaggtcaagcacagggaaccttgtagagtggttcttacttaggaaagcctacgaaagaaacgaagtagctccaaacaagccaagtgaagaggagtatcaaagaaggctcagggagagctacacaccnggattcgttaaagagccagaaaaggggttgtgggaaaacatagtatacctagattttagagccctatatccctcgattataattacccacaatgtttctcccgatactctaaatcttgagggatgcaagaactatgatatcgctcctcaagtaggccacaagttctgcaaggacatccctggttttataccaagtctcttgggacatttgttagaggaaagacaaaagattaagacaaaaatgaaggaaactcaagatcctatagaaaaaatactccttgactatagacaaaaagcgataaaactcttagcaaattctttctacggatattatggctatgcaaaagcaagatggtactgtaaggagtgtgctgagagcgttactgcctggggaagaaagtacatcgagttagtatggaaggagctcgaagaaaagtttggatttaaagtcctctacattgacactgatggtctctatgcaactatcccaggaggagaaagtgaggaaataaagaaaaaggctctagaatttgtaaaatacataaattcaaagctccctggactgctagagcttgaatatgaagggttttataagaggggattcttcgttacgaagaagaggtatgcagtaatagatgaagaaggaaaagtcattactcgtggtttagagatagttaggagagattggagtgaaattgcaaaagaaactcaagctagagttttggagacaatactaaaacacggagatgttgaagaagctgtgagaatagtaaaagaagtaatacaaaagcttgccaattatgaaattccaccagagaagctcgcaatatatgagcagataacaagaccattacatgagtataaggcgataggtcctcacgtagctgttgcaaagaaactagctgctaaaggagttaaaataaagccaggaatggtaattggatacatagtacttagaggcgatggtccaattagcaatagggcaattctagctgaggaatacgatcccaaaaagcacaagtatgacgcagaatattacatggagaaccaggttcttccagcggtacttaggatattggagggatttggatacagaaaggaagacctcagataccaaaagacaagacaagtcggcctaacttcctggcttaacattaaaaaatcctag

SEQ ID NO: 20 - DNA, 2328 bp, Pyrococcus furiosus; identical to SEQ ID NO: 19
  misc_feature (1159)..(1161): CCN is the codon for Proline where N = C, G, A, or T

SEQ ID NO: 21 - DNA, 2328 bp, Pyrococcus furiosus
  misc_feature (277)..(279): nnn = AGA, AGG, CGA, CGC, CGG, CGT (ALL POSSIBLE CODONS FOR ARGININE)
atgattttagatgtggattacataactgaagaaggaaaacctgttattaggctattcaaaaaagagaacggaaaatttaagatagagcatgatagaacttttagaccatacatttacgctcttctcagggatgattcaaagattgaagaagttaagaaaataacgggggaaaggcatggaaagattgtgagaattgttgatgtagagaaggttgagaaaaagtttctcggcaagcctattaccgtgtggaaactttatttggaacatccccaagatnnncccactattagagaaaaagttagagaacatccagcagttgtggacatcttcgaatacgatattccatttgcaaagagatacctcatcgacaaaggcctaataccaatggagggggaagaagagctaaagattcttgccttcgcnatagcnaccctctatcacgaaggagaagagtttggaaaaggcccaattataatgattagttatgcagatgaaaatgaagcaaaggtgattacttggaaaaacatagatcttccatacgttgaggttgtatcaagcgagagagagatgataaagagatttctcaggattatcagggagaaggatcctgacattatagttacttataatggagactcattcgcattcccatatttagcgaaaagggcagaaaaacttgggattaaattaaccattggaagagatggaagcgagcccaagatgcagagaataggcgatatgacggctgtagaagtcaagggaagaatacatttcgacttgtatcatgtaataacaaggacaataaatctcccaacatacacactagaggctgtatatgaagcaatttttggaaagccaaaggagaaggtatacgccgacgagatagcaaaagcctgggaaagtggagagaaccttgagagagttgccaaatactcgatggaagatgcaaaggcaacttatgaactcgggaaagaattccttccaatggaaattcagctttcaagattagttggacaacctttatgggatgtttcaaggtcaagcacagggaaccttgtagagtggttcttacttaggaaagcctacgaaagaaacgaagtagctccaaacaagccaagtgaagaggagtatcaaagaaggctcagggagagctacacaccnggattcgttaaagagccagaaaaggggttgtgggaaaacatagtatacctagattttagagccctatatccctcgattataattacccacaatgtttctcccgatactctaaatcttgagggatgcaagaactatgatatcgctcctcaagtaggccacaagttctgcaaggacatccctggttttataccaagtctcttgggacatttgttagaggaaagacaaaagattaagacaaaaatgaaggaaactcaagatcctatagaaaaaatactccttgactatagacaaaaagcgataaaactcttagcaaattctttctacggatattatggctatgcaaaagcaagatggtactgtaaggagtgtgctgagagcgttactgcctggggaagaaagtacatcgagttagtatggaaggagctcgaagaaaagtttggatttaaagtcctctacattgacactgatggtctctatgcaactatcccaggaggagaaagtgaggaaataaagaaaaaggctctagaatttgtaaaatacataaattcaaagctccctggactgctagagcttgaatatgaagggttttataagaggggattcttcgttacgaagaagaggtatgcagtaatagatgaagaaggaaaagtcattactcgtggtttagagatagttaggagagattggagtgaaattgcaaaagaaactcaagctagagttttggagacaatactaaaacacggagatgttgaagaagctgtgagaatagtaaaagaagtaatacaaaagcttgccaattatgaaattccaccagagaagctcgcaatatatgagcagataacaagaccattacatgagtataaggcgataggtcctcacgtagctgttgcaaagaaactagctgctaaaggagttaaaataaagccaggaatggtaattggatacatagtacttagaggcgatggtccaattagcaatagggcaattctagctgaggaatacgatcccaaaaagcacaagtatgacgcagaatattacatggagaaccaggttcttccagcggtacttaggatattggagggatttggatacagaaaggaagacctcagataccaaaagacaagacaagtcggcctaacttcctggcttaacattaaaaaatcctag

SEQ ID NO: 22 - DNA, 2328 bp, Pyrococcus furiosus; identical to SEQ ID NO: 21
  misc_feature (277)..(279): nnn = GAA, GAG (ALL CODONS FOR GLUTAMIC ACID)

SEQ ID NO: 23 - DNA, 2325 bp, Pyrococcus sp.
  misc_feature (277)..(279): nnn = AGA, AGG, CGA, CGC, CGG, CGT (ALL POSSIBLE CODONS FOR ARGININE)
atgatcctcgacactgactacataaccgaggatggaaagcctgtcataagaattttcaagaaggaaaacggcgagtttaagattgagtacgaccggacttttgaaccctacttctacgccctcctgaaggacgattctgccattgaggaagtcaagaagataaccgccgagaggcacgggacggttgtaacggttaagcgggttgaaaaggttcagaagaagttcctcgggagaccagttgaggtctggaaactctactttactcatccgcaggacnnnccagcgataagggacaagatacgagagcatccagcagttattgacatctacgagtacgacatacccttcgccaagcgctacctcatagacaagggattagtgccaatggaaggcgacgaggagctgaaaatgctcgccttcgacattgaaactctctaccatgagggcgaggagttcgccgaggggccaatccttatgataagctacgccgacgaggaaggggccagggtgataacttggaagaacgtggatctcccctacgttgacgtcgtctcgacggagagggagatgataaagcgcttcctccgtgttgtgaaggagaaagacccggacgttctcataacctacaacggcgacaacttcgacttcgcctatctgaaaaagcgctgtgaaaagctcggaataaacttcgccctcggaagggatggaagcgagccgaagattcagaggatgggcgacaggtttgccgtcgaagtgaagggacggatacacttcgatctctatcctgtgataagacggacgataaacctgcccacatacacgcttgaggccgtttatgaagccgtcttcggtcagccgaaggagaaggtttacgctgaggaaataaccacagcctgggaaaccggcgagaaccttgagagagtcgcccgctactcgatggaagatgcgaaggtcacatacgagcttgggaaggagttccttccgatggaggcccagctttctcgcttaatcggccagtccctctgggacgtctcccgctccagcactggcaacctcgttgagtggttcctcctcaggaaggcctatgagaggaatgagctggccccgaacaagcccgatgaaaaggagctggccagaagacggcagagctatgaaggaggctatgtaaaagagcccgagagagggttgtgggagaacatagtgtacctagattttagatccctgtacccctcaatcatcatcacccacaacgtctcgccggatacgctcaacagagaaggatgcaaggaatatgacgttgccccacaggtcggccaccgcttctgcaaggacttcccaggatttatcccgagcctgcttggagacctcctagaggagaggcagaagataaagaagaagatgaaggccacgattgacccgatcgagaggaagctcctcgattacaggcagagggccatcaagatcctggcaaacagctactacggttactacggctatgcaagggcgcgctggtactgcaaggagtgtgcagagagcgtaacggcctggggaagggagtacataacgatgaccatcaaggagatagaggaaaagtacggctttaaggtaatctacagcgacaccgacggattttttgccacaatacctggagccgatgctgaaaccgtcaaaaagaaggctatggagttcctcaagtatatcaacgccaaacttccgggcgcgcttgagctcgagtacgagggcttctacaaacgcggcttcttcgtcacgaagaagaagtatgcggtgatagacgaggaaggcaagataacaacgcgcggacttgagattgtgaggcgtgactggagcgagatagcgaaagagacgcaggcgagggttcttgaagctttgctaaaggacggtgacgtcgagaaggccgtgaggatagtcaaagaagttaccgaaaagctgagcaagtacgaggttccgccggagaagctggtgatccacgagcagataacgagggatttaaaggactacaaggcaaccggtccccacgttgccgttgccaagaggttggccgcgagaggagtcaaaatacgccctggaacggtgataagctacatcgtgctcaagggctctgggaggataggcgacagggcgataccgttcgacgagttcgacccgacgaagcacaagtacgacgccgagtactacattgagaaccaggttctcccagccgttgagagaattctgagagccttcggttaccgcaaggaagacctgcgctaccagaagacgagacaggttggtttgagtgcttggctgaagccgaagggaacttga

SEQ ID NO: 24 - DNA, 2325 bp, Pyrococcus sp.; identical to SEQ ID NO: 23
  misc_feature (277)..(279): nnn = GAA, GAG (ALL CODONS FOR GLUTAMIC ACID)

SEQ ID NO: 25 - DNA, 2325 bp, Thermococcus litoralis
  misc_feature (277)..(279): nnn = AGA, AGG, CGA, CGC, CGG, CGT (ALL POSSIBLE CODONS FOR ARGININE)
atgatactggacactgattacataacaaaagatggcaagcctataatccgaatttttaagaaagagaacggggagtttaaaatagaacttgaccctcattttcagccctatatatatgctcttctcaaagatgactccgctattgaggagataaaggcaataaagggcgagagacatggaaaaactgtgagagtgctcgatgcagtgaaagtcaggaaaaaatttttgggaagggaagttgaagtctggaagctcattttcgagcatccccaagacnnnccagctatgcggggcaaaataagggaacatccagctgtggttgacatttacgaatatgacataccctttgccaagcgttatctcatagacaagggcttgattcccatggagggagacgaggagcttaagctccttgcctttgatattgaaacgttttatcatgagggagatgaatttggaaagggcgagataataatgattagttatgccgatgaagaagaggccagagtaatcacatggaaaaatatcgatttgccgtatgtcgatgttgtgtccaatgaaagagaaatgataaagcgttttgttcaagttgttaaagaaaaagaccccgatgtgataataacttacaatggggacaattttgatttgccgtatctcataaaacgggcagaaaagctgggagttcggcttgtcttaggaagggacaaagaacatcccgaacccaagattcagaggatgggtgatagttttgctgtggaaatcaagggtagaatccactttgatcttttcccagttgtgcgaaggacgataaacctcccaacgtatacgcttgaggcagtttatgaagcagttttaggaaaaaccaaaagcaaattaggagcagaggaaattgccgctatatgggaaacagaagaaagcatgaaaaaactagcccagtactcaatggaagatgctagggcaacgtatgagctcgggaaggaattcttccccatggaagctgagctggcaaagctgataggtcaaagtgtatgggacgtctcgagatcaagcaccggcaacctcgtggagtggtatcttttaagggtggcatacgcgaggaatgaacttgcaccgaacaaacctgatgaggaagagtataaacggcgcttaagaacaacttacctgggaggatatgtaaaagagccagaaaaaggtttgtgggaaaatatcatttatttggatttccgcagtctgtacccttcaataatagttactcacaacgtatccccagatacccttgaaaaagagggctgtaagaattacgatgttgctccgatagtaggatataggttctgcaaggactttccgggctttattccctccatactcggggacttaattgcaatgaggcaagatataaagaagaaaatgaaatccacaattgacccgatcgaaaagaaaatgctcgattataggcaaagggctattaaattgcttgcaaacagctattacggctatatggggtatcctaaggcaagatggtactcgaaggaatgtgctgaaagcgttaccgcatgggggagacactacatagagatgacgataagagaaatagaggaaaagttcggctttaaggttctttatgcggacactgacggcttttatgccacaatacccggggaaaagcctgaactcattaaaaagaaagccaaggaattcctaaactacataaactccaaacttccaggtctgcttgagcttgagtatgagggcttttacttgagaggattctttgttacaaaaaagcgctatgcagtcatagatgaagagggcaggataacaacaaggggcttggaagtagtaaggagagattggagtgagatagctaaggagactcaggcaaaggttttagaggctatacttaaagagggaagtgttgaaaaagctgtagaagttgttagagatgttgtagagaaaatagcaaaatacagggttccacttgaaaagcttgttatccatgagcagattaccagggatttaaaggactacaaagccattggccctcatgtcgcgatagcaaaaagacttgccgcaagagggataaaagtgaaaccgggcacaataataagctatatcgttctcaaagggagcggaaagataagcgatagggtaattttacttacagaatacgatcctagaaaacacaagtacgatccggactactacatagaaaaccaagttttgccggcagtacttaggatactcgaagcgtttggatacagaaaggaggatttaaggtatcaaagctcaaaacaaaccggcttagatgcatggctcaagaggtag

SEQ ID NO: 26 - DNA, 2325 bp, Thermococcus litoralis; identical to SEQ ID NO: 25
  misc_feature (277)..(279): nnn = GAA, GAG (ALL CODONS FOR GLUTAMIC ACID)

SEQ ID NO: 27 - DNA, 2328 bp, Pyrococcus sp.
  misc_feature (277)..(279): nnn = AGA, AGG, CGA, CGC, CGG, CGT (ALL POSSIBLE CODONS FOR ARGININE)
atgatacttgacgctgactacatcaccgaggatgggaagccgattataaggattttcaagaaagaaaacggcgagtttaaggttgagtacgacagaaactttagaccttacatttacgctctcctcaaagatgactcgcagattgatgaggttaggaagataaccgccgagaggcatgggaagatagtgagaattatagatgccgaaaaggtaaggaagaagttcctggggaggccgattgaggtatggaggctgtactttgaacaccctcaggacnnncccgcaataagggataagataagagagcattccgcagttattgacatctttgagtacgacattccgttcgcgaagaggtacctaatagacaaaggcctaattccaatggaaggcgatgaagagctcaagttgctcgcatttgacatagaaaccctctatcacgaaggggaggagttcgcgaaggggcccattataatgataagctatgctgatgaggaagaagccaaagtcataacgtggaaaaagatcgatctcccgtacgtcgaggtagtttccagcgagagggagatgataaagcggttcctcaaggtgataagggagaaagatcccgatgttataattacctacaacggcgattctttcgaccttccctatctagttaagagggccgaaaagctcgggataaagctacccctgggaagggacggtagtgagccaaagatgcagaggcttggggatatgacagcggtggagataaagggaaggatacactttgacctctaccacgtgattaggagaacgataaacctcccaacatacaccctcgaggcagtttatgaggcaatcttcggaaagccaaaggagaaagtttacgctcacgagatagctgaggcctgggagactggaaagggactggagagagttgcaaagtattcaatggaggatgcaaaggtaacgtacgagctcggtagggagttcttcccaatggaggcccagctttcaaggttagtcggccagcccctgtgggatgtttctaggtcttcaactggcaacttggtggagtggtacctcctcaggaaggcctacgagaggaatgaattggctccaaacaagccggatgagagggagtacgagagaaggctaagggagagctacgctgggggatacgttaaggagccggagaaagggctctgggaggggttagtttccctagatttcaggagcctgtacccctcgataataatcacccataacgtctcaccggatacgctgaacagggaagggtgtagggaatacgatgtcgccccagaggttgggcacaagttctgcaaggacttcccggggtttatccccagcctgctcaagaggttattggatgaaaggcaagaaataaaaaggaagatgaaagcttctaaagacccaatcgagaagaagatgcttgattacaggcaacgggcaatcaaaatcctggcaaacagctattatgggtattatgggtacgcaaaagcccgttggtactgtaaggagtgcgcagagagcgttacggcctgggggagggaatatatagagttcgtaaggaaggaactggaggaaaagttcgggttcaaagtcttatacatagacacagatggactctacgccacaattcctggggcaaaacccgaggagataaagaagaaagccctagagttcgtagattatataaacgccaagctcccagggctgttggagcttgagtacgagggcttctacgtgagagggttcttcgtgacgaagaagaagtatgcgttgatagatgaggaagggaagataatcactagggggcttgaaatagtcaggagggactggagcgaaatagccaaagaaacccaagcaaaagtcctagaggctatcctaaagcatggcaacgttgaggaggcagtaaagatagttaaggaggtaactgaaaagctgagcaagtacgaaatacctccagaaaagctagttatttacgagcagatcacgaggccccttcacgagtacaaggctataggtccgcacgttgccgtggcaaaaaggttagccgctagaggagtaaaggtgaggcctggcatggtgatagggtacatagtgctgaggggagacgggccaataagcaagagggctatccttgcagaggagttcgatctcaggaagcataagtatgacgctgagtattacatagaaaatcaggttttacctgccgttcttagaatattagaggcctttgggtacaggaaagaagacctcaggtggcagaagactaaacagacaggtcttacggcatggcttaacatcaagaagaagtaa

SEQ ID NO: 28 - DNA, 2328 bp, Pyrococcus sp.; identical to SEQ ID NO: 27
  misc_feature (277)..(279): nnn = GAA, GAG (ALL CODONS FOR GLUTAMIC ACID)

SEQ ID NO: 29 - DNA, 2331 bp, Thermococcus sp.
  misc_feature (277)..(279): nnn = AGA, AGG, CGA, CGC, CGG, CGT (ALL POSSIBLE CODONS FOR ARGININE)
atgatccttgacgttgattacatcaccgagaatggaaagcccgtcatcagggtcttcaagaaggagaacggcgagttcaggattgaatacgaccgcgagttcgagccctacttctacgcgctcctcagggacgactctgccatcgaagaaatcaaaaagataaccgcggagaggcacggcagggtcgttaaggttaagcgcgcggagaaggtgaagaaaaagttcctcggcaggtctgtggaggtctgggtcctctacttcacgcacccgcaggacnnnccggcaatccgcgacaaaataaggaagcaccccgcggtcatcgacatctacgagtacgacatacccttcgccaagcgctacctcatagacaagggcctaatcccgatggaaggtgaggaagagcttaaactcatgtccttcgacatcgagacgctctaccacgagggagaagagtttggaaccgggccgattctgatgataagctacgccgatgaaagcgaggcgcgcgtgataacctggaagaagatcgaccttccttacgttgaggttgtctccaccgagaaggagatgattaagcgcttcttgagggtcgttaaggagaaggacccggacgtgctgataacatacaacggcgacaacttcgacttcgcctacctgaaaaagcgctgtgagaagcttggcgtgagctttaccctcgggagggacgggagcgagccgaagatacagcgcatgggggacaggtttgcggtcgaggtgaagggcagggtacacttcgacctttatccagtcataaggcgcaccataaacctcccgacctacacccttgaggctgtatacgaggcggttttcggcaagcccaaggagaaggtctacgccgaggagatagccaccgcctgggagaccggcgaggggcttgagagggtcgcgcgctactcgatggaggacgcgagggttacctacgagcttggcagggagttcttcccgatggaggcccagctttccaggctcatcggccaaggcctctgggacgtttcccgctccagcaccggcaacctcgtcgagtggttcctcctaaggaaggcctacgagaggaacgaactcgctcccaacaagcccgacgagagggagctggcgaggagaagggggggctacgccggtggctacgtcaaggagccggagcggggactgtgggacaatatcgtgtatctagactttcgtagtctctacccttcaatcataatcacccacaacgtctcgccagatacgctcaaccgcgaggggtgtaggagctacgacgttgcccccgaggtcggtcacaagttctgcaaggacttccccggcttcattccgagcctgctcggaaacctgctggaggaaaggcagaagataaagaggaagatgaaggcaactctcgacccgctggagaagaatctcctcgattacaggcaacgcgccatcaagattctcgccaacagctactacggctactacggctatgccagggcaagatggtactgcagggagtgcgccgagagcgttacggcatggggaagggagtacatcgaaatggtcatcagagagcttgaggaaaagttcggttttaaagtcctctatgcagacacagacggtctccatgccaccattcctggagcggacgctgaaacagtcaagaaaaaggcaatggagttcttaaactatatcaatcccaaactgcccggccttctcgaactcgaatacgagggcttctacgtcaggggcttcttcgtcacgaagaaaaagtacgcggtcatcgacgaggagggcaagataaccacgcgcgggcttgagatagtcaggcgcgactggagcgagatagcgaaggagacgcaggcgagggttttggaggcgatactcaggcacggtgacgttgaagaggccgtcagaattgtcagggaagtcaccgaaaagctgagcaagtacgaggttccgccggagaagctggttatccacgagcagataacgcgcgagctcaaggactacaaggccaccggcccgcacgtagccatagcgaagcgtttggccgccagaggtgttaaaatccggcccggaactgtgataagctacatcgttctgaagggctccggaaggataggcgacagggcgattcccttcgacgagttcgacccgacgaagcacaagtacgatgcggactactacatcgagaaccaggttctgccggcagttgagagaatcctcagggccttcggctaccgcaaggaagacctgcgctaccagaagacgaggcaggtcgggcttggcgcgtggctgaagccgaaggggaagaagaagtga

SEQ ID NO: 30 - DNA, 2331 bp, Thermococcus sp.; identical to SEQ ID NO: 29
  misc_feature (277)..(279): nnn = GAA, GAG (all codons FOR GLUTAMIC ACID)

SEQ ID NO: 31 - protein, 775 aa, Pyrococcus furiosus
MILDVDYITEEGKPVIRLFKKENGKFKIEHDRTFRPYIYALLRDDSKIEEVKKITGERHGKIVRIVDVEKVEKKFLGKPITVWKLYLEHPQDRPTIREKVREHPAVVDIFEYDIPFAKRYLIDKGLIPMEGEEELKILAFDIETLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLEAVYEAIFGKPKEKVYADEIAKAWESGENLERVAKYSMEDAKATYELGKEFLPMEIQLSRLVGQPLWDVSRSSTGNLVEWFLLRKAYERNEVAPNKPSEEEYQRRLRESYTGGFVKEPEKGLWENIVYLDFRALYPSIIITHNVSPDTLNLEGCKNYDIAPQVGHKFCKDIPGFIPSLLGHLLEERQKIKTKMKETQDPIEKILLDYRQKAIKLLANSFYGYYGYAKARWYCKECAESVTAWGRKYIELVWKELEEKFGFKVLYIDTDGLYATIPGGESEEIKKKALEFVKYINSKLPGLLELEYEGFYKRGFFVTKKRYAVIDEEGKVITRGLEIVRRDWSEIAKETQARVLETILKHGDVEEAVRIVKEVIQKLANYEIPPEKLAIYEQITRPLHEYKAIGPHVAVAKKLAAKGVKIKPGMVIGYIVLRGDGPISNRAILAEEYDPKKHKYDAEYYIENQVLPAVLRILEGFGYRKEDLRYQKTRQVGLTSWLNIKKS

SEQ ID NO: 32 - protein, 775 aa, Pyrococcus furiosus
MILDVDYITEEGKPVIRLFKKENGKFKIEHDRTFRPYIYALLRDDSKIEEVKKITGERHGKIVRIVDVEKVEKKFLGKPITVWKLYLEHPQDEPTIREKVREHPAVVDIFEYDIPFAKRYLIDKGLIPMEGEEELKILAFDIETLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLEAVYEAIFGKPKEKVYADEIAKAWESGENLERVAKYSMEDAKATYELGKEFLPMEIQLSRLVGQPLWDVSRSSTGNLVEWFLLRKAYERNEVAPNKPSEEEYQRRLRESYTGGFVKEPEKGLWENIVYLDFRALYPSIIITHNVSPDTLNLEGCKNYDIAPQVGHKFCKDIPGFIPSLLGHLLEERQKIKTKMKETQDPIEKILLDYRQKAIKLLANSFYGYYGYAKARWYCKECAESVTAWGRKYIELVWKELEEKFGFKVLYIDTDGLYATIPGGESEEIKKKALEFVKYINSKLPGLLELEYEGFYKRGFFVTKKRYAVIDEEGKVITRGLEIVRRDWSEIAKETQARVLETILKHGDVEEAVRIVKEVIQKLANYEIPPEKLAIYEQITRPLHEYKAIGPHVAVAKKLAAKGVKIKPGMVIGYIVLRGDGPISNRAILAEEYDPKKHKYDAEYYIENQVLPAVLRILEGFGYRKEDLRYQKTRQVGLTSWLNIKKS

SEQ ID NO: 33 - protein, 775 aa, Pyrococcus furiosus
MILDVDYITEEGKPVIRLFKKENGKFKIEHDRTFRPYIYALLRDDSKIEEVKKITGERHGKIVRIVDVEKVEKKFLGKPITVWKLYLEHPQDRPTIREKVREHPAVVDIFEYDIPFAKRYLIDKGLIPMEGEEELKILAFDIETLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLEAVYEAIFGKPKEKVYADEIAKAWESGENLERVAKYSMEDAKATYELGKEFLPMEIQLSRLVGQPLWDVSRSSTGNLVEWFLLRKAYERNEVAPNKPSEEEYQRRLRESYTPGFVKEPEKGLWENIVYLDFRALYPSIIITHNVSPDTLNLEGCKNYDIAPQVGHKFCKDIPGFIPSLLGHLLEERQKIKTKMKETQDPIEKILLDYRQKAIKLLANSFYGYYGYAKARWYCKECAESVTAWGRKYIELVWKELEEKFGFKVLYIDTDGLYATIPGGESEEIKKKALEFVKYINSKLPGLLELEYEGFYKRGFFVTKKRYAVIDEEGKVITRGLEIVRRDWSEIAKETQARVLETILKHGDVEEAVRIVKEVIQKLANYEIPPEKLAIYEQITRPLHEYKAIGPHVAVAKKLAAKGVKIKPGMVIGYIVLRGDGPISNRAILAEEYDPKKHKYDAEYYIENQVLPAVLRILEGFGYRKEDLRYQKTRQVGLTSWLNIKKS

SEQ ID NO: 34 - protein, 775 aa, Pyrococcus furiosus
MILDVDYITEEGKPVIRLFKKENGKFKIEHDRTFRPYIYALLRDDSKIEEVKKITGERHGKIVRIVDVEKVEKKFLGKPITVWKLYLEHPQDRPTIREKVREHPAVVDIFEYDIPFAKRYLIDKGLIPMEGEEELKILAFAIATLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLEAVYEAIFGKPKEKVYADEIAKAWESGENLERVAKYSMEDAKATYELGKEFLPMEIQLSRLVGQPLWDVSRSSTGNLVEWFLLRKAYERNEVAPNKPSEEEYQRRLRESYTGGFVKEPEKGLWENIVYLDFRALYPSIIITHNVSPDTLNLEGCKNYDIAPQVGHKFCKDIPGFIPSLLGHLLEERQKIKTKMKETQDPIEKILLDYRQKAIKLLANSFYGYYGYAKARWYCKECAESVTAWGRKYIELVWKELEEKFGFKVLYIDTDGLYATIPGGESEEIKKKALEFVKYINSKLPGLLELEYEGFYKRGFFVTKKRYAVIDEEGKVITRGLEIVRRDWSEIAKETQARVLETILKHGDVEEAVRIVKEVIQKLANYEIPPEKLAIYEQITRPLHEYKAIGPHVAVAKKLAAKGVKIKPGMVIGYIVLRGDGPISNRAILAEEYDPKKHKYDAEYYIENQVLPAVLRILEGFGYRKEDLRYQKTRQVGLTSWLNIKKS

SEQ ID NO: 35 - protein, 775 aa, Pyrococcus furiosus
MILDVDYITEEGKPVIRLFKKENGKFKIEHDRTFRPYIYALLRDDSKIEEVKKITGERHGKIVRIVDVEKVEKKFLGKPITVWKLYLEHPQDEPTIREKVREHPAVVDIFEYDIPFAKRYLIDKGLIPMEGEEELKILAFDIETLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLEAVYEAIFGKPKEKVYADEIAKAWESGENLERVAKYSMEDAKATYELGKEFLPMEIQLSRLVGQPLWDVSRSSTGNLVEWFLLRKAYERNEVAPNKPSEEEYQRRLRESYTPGFVKEPEKGLWENIVYLDFRALYPSIIITHNVSPDTLNLEGCKNYDIAPQVGHKFCKDIPGFIPSLLGHLLEERQKIKTKMKETQDPIEKILLDYRQKAIKLLANSFYGYYGYAKARWYCKECAESVTAWGRKYIELVWKELEEKFGFKVLYIDTDGLYATIPGGESEEIKKKALEFVKYINSKLPGLLELEYEGFYKRGFFVTKKRYAVIDEEGKVITRGLEIVRRDWSEIAKETQARVLETILKHGDVEEAVRIVKEVIQKLANYEIPPEKLAIYEQITRPLHEYKAIGPHVAVAKKLAAKGVKIKPGMVIGYIVLRGDGPISNRAILAEEYDPKKHKYDAEYYIENQVLPAVLRILEGFGYRKEDLRYQKTRQVGLTSWLNIKKS

SEQ ID NO: 36 - protein, 775 aa, Pyrococcus furiosus; identical to SEQ ID NO: 34

SEQ ID NO: 37 - protein, 775 aa, Pyrococcus sp.
MILDADYITEDGKPIIRIFKKENGEFKVEYDRNFRPYIYALLKDDSQIDEVRKITAERHGKIVRIIDAEKVRKKFLGRPIEVWRLYFEHPQDRPAIRDKIREHSAVIDIFEYDIPFAKRYLIDKGLIPMEGDEELKLLAFDIETLYHEGEEFAKGPIIMISYADEEEAKVITWKKIDLPYVEVVSSEREMIKRFLKVIREKDPDVIITYNGDSFDLPYLVKRAEKLGIKLPLGRDGSEPKMQRLGDMTAVEIKGRIHFDLYHVIRRTINLPTYTLEAVYEAIFGKPKEKVYAHEIAEAWETGKGLERVAKYSMEDAKVTYELGREFFPMEAQLSRLVGQPLWDVSRSSTGNLVEWYLLRKAYERNELAPNKPDEREYERRLRESYAGGYVKEPEKGLWEGLVSLDFRSLYPSIIITHNVSPDTLNREGCREYDVAPEVGHKFCKDFPGFIPSLLKRLLDERQEIKRKMKASKDPIEKKMLDYRQRAIKILANSYYGYYGYAKARWYCKECAESVTAWGREYIEFVRKELEEKFGFKVLYIDTDGLYATIPGAKPEEIKKKALEFVDYINAKLPGLLELEYEGFYVRGFFVTKKKYALIDEEGKIITRGLEIVRRDWSEIAKETQAKVLEAILKHGNVEEAVKIVKEVTEKLSKYEIPPEKLVIYEQITRPLHEYKAIGPHVAVAKRLAARGVKVRPGMVIGYIVLRGDGPISKRAILAEEFDLRKHKYDAEYYIENQVLPAVLRILEAFGYRKEDLRWQKTKQTGLTAWLNIKKK

SEQ ID NO: 38 - protein, 775 aa, Pyrococcus sp.
MILDADYITEDGKPIIRIFKKENGEFKVEYDRNFRPYIYALLKDDSQIDEVRKITAERHGKIVRIIDAEKVRKKFLGRPIEVWRLYFEHPQDEPAIRDKIREHSAVIDIFEYDIPFAKRYLIDKGLIPMEGDEELKLLAFDIETLYHEGEEFAKGPIIMISYADEEEAKVITWKKIDLPYVEVVSSEREMIKRFLKVIREKDPDVIITYNGDSFDLPYLVKRAEKLGIKLPLGRDGSEPKMQRLGDMTAVEIKGRIHFDLYHVIRRTINLPTYTLEAVYEAIFGKPKEKVYAHEIAEAWETGKGLERVAKYSMEDAKVTYELGREFFPMEAQLSRLVGQPLWDVSRSSTGNLVEWYLLRKAYERNELAPNKPDEREYERRLRESYAGGYVKEPEKGLWEGLVSLDFRSLYPSIIITHNVSPDTLNREGCREYDVAPEVGHKFCKDFPGFIPSLLKRLLDERQEIKRKMKASKDPIEKKMLDYRQRAIKILANSYYGYYGYAKARWYCKECAESVTAWGREYIEFVRKELEEKFGFKVLYIDTDGLYATIPGAKPEEIKKKALEFVDYINAKLPGLLELEYEGFYVRGFFVTKKKYALIDEEGKIITRGLEIVRRDWSEIAKETQAKVLEAILKHGNVEEAVKIVKEVTEKLSKYEIPPEKLVIYEQITRPLHEYKAIGPHVAVAKRLAARGVKVRPGMVIGYIVLRGDGPISKRAILAEEFDLRKHKYDAEYYIENQVLPAVLRILEAFGYRKEDLRWQKTKQTGLTAWLNIKKK

SEQ ID NO: 39 - protein, 773 aa, Thermococcus gorgonarius
MILDTDYITEDGKPVIRIFKKENGEFKIDYDRNFEPYIYALLKDDSAIEDVKKITAERHGTTVRVVRAEKVKKKFLGRPIEVWKLYFTHPQDRPAIRDKIKEHPAVVDIYEYDIPFAKRYLIDKGLIPMEGDEELKMLAFDIETLYHEGEEFAEGPILMISYADEEGARVITWKNIDLPYVDVVSTEKEMIKRFLKVVKEKDPDVLITYNGDNFDFAYLKKRSEKLGVKFILGREGSEPKIQRMGDRFAVEVKGRIHFDLYPVIRRTINLPTYTLEAVYEAIFGQPKEKVYAEEIAQAWETGEGLERVARYSMEDAKVTYELGKEFFPMEAQLSRLVGQSLWDVSRSSTGNLVEWFLLRKAYERNELAPNKPDERELARRRESYAGGYVKEPERGLWENIVYLDFRSLYPSIIITHNVSPDTLNREGCEEYDVAPQVGHKFCKDFPGFIPSLLGDLLEERQKVKKKMKATIDPIEKKLLDYRQRAIKILANSFYGYYGYAKARWYCKECAESVTAWGRQYIETTIREIEEKFGFKVLYADTDGFFATIPGADAETVKKKAKEFLDYINAKLPGLLELEYEGFYKRGFFVTKKKYAVIDEEDKITTRGLEIVRRDWSEIAKETQARVLEAILKHGDVEEAVRIVKEVTEKLSKYEVPPEKLVIYEQITRDLKDYKATGPHVAVAKRLAARGIKIRPGTVISYIVLKGSGRIGDRAIPFDEFDPAKHKYDAEYYIENQVLPAVERILRAFGYRKEDLRYQKTRQVGLGAWLKPKT

SEQ ID NO: 40 - protein, 773 aa, Thermococcus gorgonarius
MILDTDYITEDGKPVIRIFKKENGEFKIDYDRNFEPYIYALLKDDSAIEDVKKITAERHGTTVRVVRAEKVKKKFLGRPIEVWKLYFTHPQDEPAIRDKIKEHPAVVDIYEYDIPFAKRYLIDKGLIPMEGDEELKMLAFDIETLYHEGEEFAEGPILMISYADEEGARVITWKNIDLPYVDVVSTEKEMIKRFLKVVKEKDPDVLITYNGDNFDFAYLKKRSEKLGVKFILGREGSEPKIQRMGDRFAVEVKGRIHFDLYPVIRRTINLPTYTLEAVYEAIFGQPKEKVYAEEIAQAWETGEGLERVARYSMEDAKVTYELGKEFFPMEAQLSRLVGQSLWDVSRSSTGNLVEWFLLRKAYERNELAPNKPDERELARRRESYAGGYVKEPERGLWENIVYLDFRSLYPSIIITHNVSPDTLNREGCEEYDVAPQVGHKFCKDFPGFIPSLLGDLLEERQKVKKKMKATIDPIEKKLLDYRQRAIKILANSFYGYYGYAKARWYCKECAESVTAWGRQYIETTIREIEEKFGFKVLYADTDGFFATIPGADAETVKKKAKEFLDYINAKLPGLLELEYEGFYKRGFFVTKKKYAVIDEEDKITTRGLEIVRRDWSEIAKETQARVLEAILKHGDVEEAVRIVKEVTEKLSKYEVPPEKLVIYEQITRDLKDYKATGPHVAVAKRLAARGIKIRPGTVISYIVLKGSGRIGDRAIPFDEFDPAKHKYDAEYYIENQVLPAVERILRAFGYRKEDLRYQKTRQVGLGAWLKPKT

SEQ ID NO: 41 - protein, 774 aa, Pyrococcus sp.
MILDTDYITEDGKPVIRIFKKENGEFKIEYDRTFEPYFYALLKDDSAIEEVKKITAERHGTVVTVKRVEKVQKKFLGRPVEVWKLYFTHPQDRPAIRDKIREHGAVIDIYEYDIPFAKRYLIDKGLVPMEGDEELKMLAFDIQTLYHEGEEFAEGPILMISYADEEGARVITWKNVDLPYVDVVSTEREMIKRFLRVVKEKDPDVLITYNGDNFDFAYLKKRCEKLGINFALGRDGSEPKIQRMGDRFAVEVKGRIHFDLYPVIRRTINLPTYTLEAVYEAVFGQPKEKVYAEEITPAWETGENLERVARYSMEDAKVTYELGKEFLPMEAQLSRLIGQSLWDVSRSSTGNLVEWFLLRKAYERNELAPNKPDEKELARRRQSYEGGYVKEPERGLWENIVYLDFRSLYPSIIITHNVSPDTLNREGCKEYDVAPQVGHRFCKDFPGFIPSLLGDLLEERQKIKKKMKATIDPIERKLLDYRQRAIKILANSYYGYYGYARARWYCKECAESVTAWGREYITMTIKEIEEKYGFKVIYSDTDGFFATIPGADAETVKKKAMEFLNYINAKLPGALELEYEGFYKRGFFVTKKKYAVIDEEGKITTRGLEIVRRDWSEIAKETQARVLEALLKDGDVEKAVRIVKEVTEKLSKYEVPPEKLVIHEQITRDLKDYKATGPHVAVAKRLAARGVKIRPGTVISYIVLKGSGRIGDRAIPFDEFDPTKHKYDAEYYIENQVLPAVERILRAFGYRKEDLRYQKTRQVGLSAWLKPKGT

SEQ ID NO: 42 - protein, 774 aa, Pyrococcus sp.
MILDTDYITEDGKPVIRIFKKENGEFKIEYDRTFEPYFYALLKDDSAIEEVKKITAERHGTVVTVKRVEKVQKKFLGRPVEVWKLYFTHPQDEPAIRDKIREHGAVIDIYEYDIPFAKRYLIDKGLVPMEGDEELKMLAFDIQTLYHEGEEFAEGPILMISYADEEGARVITWKNVDLPYVDVVSTEREMIKRFLRVVKEKDPDVLITYNGDNFDFAYLKKRCEKLGINFALGRDGSEPKIQRMGDRFAVEVKGRIHFDLYPVIRRTINLPTYTLEAVYEAVFGQPKEKVYAEEITPAWETGENLERVARYSMEDAKVTYELGKEFLPMEAQLSRLIGQSLWDVSRSSTGNLVEWFLLRKAYERNELAPNKPDEKELARRRQSYEGGYVKEPERGLWENIVYLDFRSLYPSIIITHNVSPDTLNREGCKEYDVAPQVGHRFCKDFPGFIPSLLGDLLEERQKIKKKMKATIDPIERKLLDYRQRAIKILANSYYGYYGYARARWYCKECAESVTAWGREYITMTIKEIEEKYGFKVIYSDTDGFFATIPGADAETVKKKAMEFLNYINAKLPGALELEYEGFYKRGFFVTKKKYAVIDEEGKITTRGLEIVRRDWSEIAKETQARVLEALLKDGDVEKAVRIVKEVTEKLSKYEVPPEKLVIHEQITRDLKDYKATGPHVAVAKRLAARGVKIRPGTVISYIVLKGSGRIGDRAIPFDEFDPTKHKYDAEYYIENQVLPAVERILRAFGYRKEDLRYQKTRQVGLSAWLKPKGT

SEQ ID NO: 43 - protein, 774 aa, Thermococcus litoralis
MILDTDYITKDGKPIIRIFKKENGEFKIELDPHFQPYIYALLKDDSAIEEIKAIKGERHGKTVRVLDAVKVRKKFLGREVEVWKLIFEHPQDRPAMRGKIREHPAVVDIYEYDIPFAKRYLIDKGLIPMEGDEELKLLAFDIETFYHEGDEFGKGEIIMISYADEEEARVITWKNIDLPYVDVVSNEREMIKRFVQVVKEKDPDVIITYNGDNFDLPYLIKRAEKLGVRLVLGRDKEHPEPKIQRMGDSFAVEIKGRIHFDLFPVVRRTINLPTYTLEAVYEAVLGKTKSKLGAEEIAAIWETEESMKKLAQYSMEDARATYELGKEFFPMEAELAKLIGQSVWDVSRSSTGNLVEWYLLRVAYARNELAPNKPDEEEYKRRLRTTYLGGYVKEPEKGLWENIIYLDFRSLYPSIIVTHNVSPDTLEKEGCKNYDVAPIVGYRFCKDFPGFIPSILGDLIAMRQDIKKKMKSTIDPIEKKMLDYRQRAIKLLANSYYGYMGYPKARWYSKECAESVTAWGRHYIEMTIREIEEKFGFKVLYADTDGFYATIPGEKPELIKKKAKEFLNYINSKLPGLLELEYEGFYLRGFFVTKKRYAVIDEEGRITTRGLEVVRRDWSEIAKETQAKVLEAILKEGSVEKAVEVVRDVVEKIAKYRVPLEKLVIHEQITRDLKDYKAIGPHVAIAKRLAARGIKVKPGTIISYIVLKGSGKISDRVILLTEYDPRKHKYDPDYYIENQVLPAVLRILEAFGYRKEDLRYQSSKQTGLDAWLKR

SEQ ID NO: 44 - protein, 774 aa, Thermococcus litoralis
MILDTDYITKDGKPIIRIFKKENGEFKIELDPHFQPYIYALLKDDSAIEEIKAIKGERHGKTVRVLDAVKVRKKFLGREVEVWKLIFEHPQDEPAMRGKIREHPAVVDIYEYDIPFAKRYLIDKGLIPMEGDEELKLLAFDIETFYHEGDEFGKGEIIMISYADEEEARVITWKNIDLPYVDVVSNEREMIKRFVQVVKEKDPDVIITYNGDNFDLPYLIKRAEKLGVRLVLGRDKEHPEPKIQRMGDSFAVEIKGRIHFDLFPVVRRTINLPTYTLEAVYEAVLGKTKSKLGAEEIAAIWETEESMKKLAQYSMEDARATYELGKEFFPMEAELAKLIGQSVWDVSRSSTGNLVEWYLLRVAYARNELAPNKPDEEEYKRRLRTTYLGGYVKEPEKGLWENIIYLDFRSLYPSIIVTHNVSPDTLEKEGCKNYDVAPIVGYRFCKDFPGFIPSILGDLIAMRQDIKKKMKSTIDPIEKKMLDYRQRAIKLLANSYYGYMGYPKARWYSKECAESVTAWGRHYIEMTIREIEEKFGFKVLYADTDGFYATIPGEKPELIKKKAKEFLNYINSKLPGLLELEYEGFYLRGFFVTKKRYAVIDEEGRITTRGLEVVRRDWSEIAKETQAKVLEAILKEGSVEKAVEVVRDVVEKIAKYRVPLEKLVIHEQITRDLKDYKAIGPHVAIAKRLAARGIKVKPGTIISYIVLKGSGKISDRVILLTEYDPRKHKYDPDYYIENQVLPAVLRILEAFGYRKEDLRYQSSKQTGLDAWLKR

SEQ ID NO: 45 - protein, 776 aa, Thermococcus sp.
MILDVDYITENGKPVIRVFKKENGEFRIEYDREFEPYFYALLRDDSAIEEIKKITAERHGRVVKVKRAEKVKKKFLGRSVEVWVLYFTHPQDRPAIRDKIRKHPAVIDIYEYDIPFAKRYLIDKGLIPMEGEEELKLMSFDIETLYHEGEEFGTGPILMISYADESEARVITWKKIDLPYVEVVSTEKEMIKRFLRVVKEKDPDVLITYNGDNFDFAYLKKRCEKLGVSFTLGRDGSEPKIQRMGDRFAVEVKGRVHFDLYPVIRRTINLPTYTLEAVYEAVFGKPKEKVYAEEIATAWETGEGLERVARYSMEDARVTYELGREFFPMEAQLSRLIGQGLWDVSRSSTGNLVEWFLLRKAYERNELAPNKPDERELARRRGGYAGGYVKEPERGLWDNIVYLDFRSLYPSIIITHNVSPDTLNREGCRSYDVAPEVGHKFCKDFPGFIPSLLGNLLEERQKIKRKMKATLDPLEKNLLDYRQRAIKILANSYYGYYGYARARWYCRECAESVTAWGREYIEMVIRELEEKFGFKVLYADTDGLHATIPGADAETVKKKAMEFLNYINPKLPGLLELEYEGFYVRGFFVTKKKYAVIDEEGKITTRGLEIVRRDWSEIAKETQARVLEAILRHGDVEEAVRIVREVTEKLSKYEVPPEKLVIHEQITRELKDYKATGPHVAIAKRLAARGVKIRPGTVISYIVLKGSGRIGDRAIPFDEFDPTKHKYDADYYIENQVLPAVERILRAFGYRKEDLRYQKTRQVGLGAWLKPKGKKK

SEQ ID NO: 46 - protein, 776 aa, Thermococcus sp.
MILDVDYITENGKPVIRVFKKENGEFRIEYDREFEPYFYALLRDDSAIEEIKKITAERHGRVVKVKRAEKVKKKFLGRSVEVWVLYFTHPQDEPAIRDKIRKHPAVIDIYEYDIPFAKRYLIDKGLIPMEGEEELKLMSFDIETLYHEGEEFGTGPILMISYADESEARVITWKKIDLPYVEVVSTEKEMIKRFLRVVKEKDPDVLITYNGDNFDFAYLKKRCEKLGVSFTLGRDGSEPKIQRMGDRFAVEVKGRVHFDLYPVIRRTINLPTYTLEAVYEAVFGKPKEKVYAEEIATAWETGEGLERVARYSMEDARVTYELGREFFPMEAQLSRLIGQGLWDVSRSSTGNLVEWFLLRKAYERNELAPNKPDERELARRRGGYAGGYVKEPERGLWDNIVYLDFRSLYPSIIITHNVSPDTLNREGCRSYDVAPEVGHKFCKDFPGFIPSLLGNLLEERQKIKRKMKATLDPLEKNLLDYRQRAIKILANSYYGYYGYARARWYCRECAESVTAWGREYIEMVIRELEEKFGFKVLYADTDGLHATIPGADAETVKKKAMEFLNYINPKLPGLLELEYEGFYVRGFFVTKKKYAVIDEEGKITTRGLEIVRRDWSEIAKETQARVLEAILRHGDVEEAVRIVREVTEKLSKYEVPPEKLVIHEQITRELKDYKATGPHVAIAKRLAARGVKIRPGTVISYIVLKGSGRIGDRAIPFDEFDPTKHKYDADYYIENQVLPAVERILRAFGYRKEDLRYQKTRQVGLGAWLKPKGKKK

SEQ ID NO: 47 - DNA, 2322 bp, Thermococcus gorgonarius
  misc_feature (277)..(279): nnn = AGA, AGG, CGA, CGC, CGG, CGT
atgatcctcgatacagactacataactgaggatggaaagcccgtcatcaggatcttcaagaaggagaacggcgagttcaccatagactacgacagaaactttgagccatacatctacgcgctcttgaaggacgactctccgattgaggacgtcaagaagataactgccgagaggcacggcactaccgttagggttgtcagggccgagaaagtgaagaagaagttcctaggcaggccgatagaggtctggaagctctacttcactcacccccaggacnnncccgcaatcagggacaagataaaggagcatcctgccgttgtggacatctacgagtacgacatccccttcgcgaagcgctacctcatagacaaaggcttaatcccgatggagggcgacgaggaacttaagatgctcgccttcgacatcgagacgctctatcacgagggcgaggagttcgccgaagggcctatcctgatgataagctacgccgacgaggaaggggcgcgcgttattacctggaagaatatcgaccttccctatgtcgacgtcgtttccaccgagaaggagatgataaagcgcttcctcaaggtcgtcaaggaaaaggatcccgacgtcctcataatctacaacggcgacaacttcgacttcgcctacctcaagaagcgctccgagaagctcggagtcaagttcatcctcggaagggaagggagcgaaccgaaaatccagcgcatgggcgatcgctttgcggtggaggtcaagggaaggattcacttcgacctctaccccgtcattaggagaacgattaacctccccacttacacccttgaggcagtatatgaagccatctttggacagccgaaggagaaggtctacgctgaggagatagcgcaggcctgggaaacgggcgagggattagaaagggtggcccgctactcgatggaggacgcgaaggtaacctatgaactcggaaaagagttcttccctatggaagcccagctctcgcgcctcgtaggccagagcctctgggatgtatctcgctcgagtaccggaaacctcgtcgagtggtttttgctgaggaaggcctacgagaggaatgaacttgcaccaaacaagccggacgagagggagctggcaagaagaagggagagctacgcgggtggatacgtcaaggagcccgaaaggggactgtgggagaacatcgtgtatctggacttccgctccctgtatccttcgataataatcacccataacgtctcccctgatacactcaacagggagggttgtgaggagtacgacgtggctcctcaggtaggccataagttctgcaaggacttccccggcttcatcccaagcctcctcggagacctcttggaggagagacagaaggtaaagaagaagatgaaggccactatagacccaatcgagaagaaactcctcgattacaggcaacgagcaatcaaaatccttgctaatagcttctacggttactacggctatacaaaggcccgctggtactacaaggagtgcgccgagagcgttaccggttggggcagggagtacatcgagaccacgataagggaaatagaggagaaatttggctttaaagtcctctacgcggacacagatggatttttcgcaacaatacctggagcggacgccgaaaccgtcaaaaagaaggcaaaggagttcctggactacatcaacgccaaactgcccggcctgctcgaactcgaatacgagggcttctacaagcgcggcttcttcgtgacgaagaagaagtacgcggttatagacgaggaggacaagataacgacgcgcgggcttgaaatagttaggcgtgactggagcgagatagcgaaggagacgcaggcgagggttcttgaggcgatactaaagcacggtgacgttgaagaagcggtaaggattgtcaaagaggttacggagaagctgagcaagtacgaggttccaccggagaagctggtcatctacgagcagataacccgcgacctgaaggactacaaggccaccgggccgcatgtggctgttgcaaaacgcctcgccgcaagggggataaaaatccggcccggaacggtcataagctacatcgtgctcaaaggctcgggaaggattggggacagggctataccctttgacgaatttgacccggcaaagcacaagtacgatgcagaatactacatcgagaaccaggttcttccagctgtggagaggattctgagggcctttggttaccgtaaagaagatttaaggtatcagaaaacgcggcaggttggcttgggggcgtggctaaaacctaagacatga

SEQ ID NO: 48 - protein, 773 aa, Thermococcus gorgonarius
  misc_feature (93)..(93): The ′Xaa′ at location 93 stands for Lys, Asn, Arg, Ser, Thr, Ile, Met, Glu, Asp, Gly, Ala, Val, Gln, His, Pro, Leu, Tyr, Trp, Cys, or Phe.
MILDTDYITEDGKPVIRIFKKENGEFTIDYDRNFEPYIYALLKDDSPIEDVKKITAERHGTTVRVVRAEKVKKKFLGRPIEVWKLYFTHPQDXPAIRDKIKEHPAVVDIYEYDIPFAKRYLIDKGLIPMEGDEELKMLAFDIETLYHEGEEFAEGPILMISYADEEGARVITWKNIDLPYVDVVSTEKEMIKRFLKVVKEKDPDVLIIYNGDNFDFAYLKKRSEKLGVKFILGREGSEPKIQRMGDRFAVEVKGRIHFDLYPVIRRTINLPTYTLEAVYEAIFGQPKEKVYAEEIAQAWETGEGLERVARYSMEDAKVTYELGKEFFPMEAQLSRLVGQSLWDVSRSSTGNLVEWFLLRKAYERNELAPNKPDERELARRRESYAGGYVKEPERGLWENIVYLDFRSLYPSIIITHNVSPDTLNREGCEEYDVAPQVGHKFCKDFPGFIPSLLGDLLEERQKVKKKMKATIDPIEKKLLDYRQRAIKILANSFYGYYGYTKARWYYKECAESVTGWGREYIETTIREIEEKFGFKVLYADTDGFFATIPGADAETVKKKAKEFLDYINAKLPGLLELEYEGFYKRGFFVTKKKYAVIDEEDKITTRGLEIVRRDWSEIAKETQARVLEAILKHGDVEEAVRIVKEVTEKLSKYEVPPEKLVIYEQITRDLKDYKATGPHVAVAKRLAARGIKIRPGTVISYIVLKGSGRIGDRAIPFDEFDPAKHKYDAEYYIENQVLPAVERILRAFGYRKEDLRYQKTRQVGLGAWLKPKT

SEQ ID NO: 49 - DNA, 2322 bp, Thermococcus gorgonarius; identical to SEQ ID NO: 47
  misc_feature (277)..(279): nnn = GAA, GAG

SEQ ID NO: 50 - protein, 773 aa, Thermococcus gorgonarius; identical to SEQ ID NO: 48
  misc_feature (93)..(93): The ′Xaa′ at location 93 stands for Lys, Asn, Arg, Ser, Thr, Ile, Met, Glu, Asp, Gly, Ala, Val, Gln, His, Pro, Leu, Tyr, Trp, Cys, or Phe.

SEQ ID NO: 51 - DNA, 3499 bp, Pyrococcus furiosus
  misc_feature (1)..(3499): n=any nucleotide
ccctggtcctgggtccacatatatgttcttactcgcctttatgaagaatcccccagtcgctctaacctgggttatagtgacaaatcttcctccaccaccgcccaagaaggttatttctatcaactctacacctcccctattttctctcttatgagatttttaagtatagttatagagaaggttttatactccaaactgagttagtagatatgtggggagcataatgattttagatgtggattacataactgaagaaggaaaacctgttattaggctattcaaaaaagagaacggaaaatttaagatagagcatgatagaacttttagaccatacatttacgctcttctcagggatgattcaaagattgaagaagttaagaaaataacgggggaaaggcatggaaagattgtgagaattgttgatgtagagaaggttgagaaaaagtttctcggcaagcctattaccgtgtggaaactttatttggaacatccccaagatgttcccactattagagaaaaagttagagaacatccagcagttgtggacatcttcgaatacgatattccatttgcaaagagatacctcatcgacaaaggcctaataccaatggagggggaagaagagctaaagattcttgccttcgatatagaaaccctctatcacgaaggagaagagtttggaaaaggcccaattataatgattagttatgcagatgaaaatgaagcaaaggtgattacttggaaaaacatagatcttccatacgttgaggttgtatcaagcgagagagagatgataaagagatttctcaggattatcagggagaaggatcctgacattatagttacttataatggagactcattcgacttcccatatttagcgaaaagggcagaaaaacttgggattaaattaaccattggaagagatggaagcgagcccaagatgcagagaataggcgatatgacggctgtagaagtcaagggaagaatacatttcgacttgtatcatgtaataacaaggacaataaatctcccaacatacacactagaggctgtatatgaagcaatttttggaaagccaaaggagaaggtatacgccgacgagatagcaaaagcctgggaaagtggagagaaccttgagagagttgccaaatactcgatggaagatgcaaaggcaacttatgaactcgggaaagaattccttccaatggaaattcagctttcaagattagttggacaacctttatgggatgtttcaaggtcaagcacagggaaccttgtagagtggttcttacttaggaaagcctacgaaagaaacgaagtagctccaaacaagccaagtgaagaggagtatcaaagaaggctcagggagagctacacaggtggattcgttaaagagccagaaaaggggttgtgggaaaacatagtatacctagattttagagccctatatccctcgattataattacccacaatgtttctcccgatactctaaatcttgagggatgcaagaactatgatatcgctcctcaagtaggccacaagttctgcaaggacatccctggttttataccaagtctcttgggacatttgttagaggaaagacaaaagattaagacaaaaatgaaggaaactcaagatcctatagaaaaaatactccttgactatagacaaaaagcgataaaactcttagcaaattctttctacggatattatggctatgcaaaagcaagatggtactgtaaggagtgtgctgagagcgttactgcctggggaagaaagtacatcgagttagtatggaaggagctcgaagaaaagtttggatttaaagtcctctacattgacactgatggtctctatgcaactatcccaggaggagaaagtgaggaaataaagaaaaaggctctagaatttgtaaaatacataaattcaaagctccctggactgctagagcttgaatatgaagggttttataagaggggattcttcgttacgaagaagaggtatgcagtaatagatgaagaaggaaaagtcattactcgtggtttagagatagttaggagagattggagtgaaattgcaaaagaaactcaagctagagttttggagacaatactaaaacacggagatgttgaagaagctgtgagaatagtaaaagaagtaatacaaaagcttgccaattatgaaattccaccagagaagctcgcaatatatgagcagataacaagaccattacatgagtataaggcgataggtcctcacgtagctgttgcaaagaaactagctgctaaaggagttaaaataaagccaggaatggtaattggatacatagtacttagaggcgatggtccaattagcaatagggcaattctagctgaggaatacgatcccaaaaagcacaagtatgacgcagaatattacattgagaaccaggttcttccagcggtacttaggatattggagggatttggatacagaaaggaagacctcagataccaaaagacaagacaagtcggcctaacttcctggcttaacattaaaaaatcctagaaaagcgatagatatcaacttttattctttctaacctttttctatgaaagaagaactgagcaggaattaccagttcttccgttattttatgggtaattaaaaacccatgctcttgggagaatcttcgaataaaatccctaacttcaggctttgctaagtgaatagaataaacaacatcactcacttcaaacgccttcgttagaaatggtctatctgcatgcttctctggctcggaannggaggattcataacaacagtatcaacattctcagagaattgagaaacatcagaaactttgacttctacaacatttctaactttgcaactcttcaagattttctaaaagaattttaacggcctcctcgtcaatttcgacgacgtagatcttttttgctccaagcagagccgctccaatggataacacccctgttcccgcacccaagtccgctacaattttttccttgtatctcctaatgtataagcaagccaaaggagagtagatgctacctttccgggagttttgtattgctctagccaaggtttgggatttttgaatcctttaactctggaaagtataatttcaagctccttcttcttcatgacagatgaaaaattgttttgtctctttttaacttttacagaaataactgtctcaaattatgacaactcttgacatttttacttcattaccagggtaatgtttttaagtatgaaatttttctttcatagaggaggnnnnnngtcctctcctcgatttccttggttgtgctccatatgataagcttccaaagtgggtgttcagacttttagacactcaaataccagacgacaatggtgtgctcactcaagccccatatgggttgagaaaagtagaagcggcactactcagatgcttccccaggaatgaggttgttgtagctcntcccngaaagattgagatgttcttgg

SEQ ID NO: 52 - protein, 775 aa, Pyrococcus furiosus
MILDVDYITEEGKPVIRLFKKENGKFKIEHDRTFRPYIYALLRDDSKIEEVKKITGERHGKIVRIVDVEKVEKKFLGKPITVWKLYLEHPQDVPTIREKVREHPAVVDIFEYDIPFAKRYLIDKGLIPMEGEEELKILAFDIETLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLEAVYEAIFGKPKEKVYADEIAKAWESGENLERVAKYSMEDAKATYELGKEFLPMEIQLSRLVGQPLWDVSRSSTGNLVEWFLLRKAYERNEVAPNKPSEEEYQRRLRESYTGGFVKEPEKGLWENIVYLDFRALYPSIIITHNVSPDTLNLEGCKNYDIAPQVGHKFCKDIPGFIPSLLGHLLEERQKIKTKMKETQDPIEKILLDYRQKAIKLLANSFYGYYGYAKARWYCKECAESVTAWGRKYIELVWKELEEKFGFKVLYIDTDGLYATIPGGESEEIKKKALEFVKYINSKLPGLLELEYEGFYKRGFFVTKKRYAVIDEEGKVITRGLEIVRRDWSEIAKETQARVLETILKHGDVEEAVRIVKEVIQKLANYEIPPEKLAIYEQITRPLHEYKAIGPHVAVAKKLAAKGVKIKPGMVIGYIVLRGDGPISNRAILAEEYDPKKHKYDAEYYIENQVLPAVLRILEGFGYRKEDLRYQKTRQVGLTSWLNIKKS

SEQ ID NO: 53 - DNA, 31 bp, Artificial sequence
  Primer
gaacatccccaagataaacccactattagag

SEQ ID NO: 54 - DNA, 31 bp, Artificial sequence
  Primer
ctctaatagtgggtttatcttggggatgttc

SEQ ID NO: 55 - DNA, 37 bp, Artificial sequence
  Primer
gaacatccccaagatgcacccactattagagaaaaag

SEQ ID NO: 56 - DNA, 37 bp, Artificial sequence
  Primer
gaacatccccaagatgaccccactattagagaaaaag

SEQ ID NO: 57 - DNA, 38 bp, Artificial sequence
  Primer
gaacatccccaagattgcccccactattagagaaaaag

SEQ ID NO: 58 - DNA, 37 bp, Artificial sequence
  Primer
gaacatccccaagatatacccactattagagaaaaag

SEQ ID NO: 59 - DNA, 37 bp, Artificial sequence
  Primer
gaacatccccaagatatgcccactattagagaaaaag

SEQ ID NO: 60 - DNA, 37 bp, Artificial sequence
  Primer
gaacatccccaagatttccccactattagagaaaaag

SEQ ID NO: 61 - DNA, 37 bp, Artificial sequence
  Primer
gaacatccccaagatcctcccactattagagaaaaag

SEQ ID NO: 62 - DNA, 37 bp, Artificial sequence
  Primer
gaacatccccaagatagccccactattagagaaaaag

SEQ ID NO: 63 - DNA, 37 bp, Artificial sequence
  Primer
gaacatccccaagatacacccactattagagaaaaag

SEQ ID NO: 64 - DNA, 37 bp, Artificial sequence
  Primer
gaacatccccaagattaccccactattagagaaaaag

SEQ ID NO: 65 - DNA, 37 bp, Artificial sequence
  Primer
gaacatccccaagattggcccactattagagaaaaag

(19) United States
(12) Patent Application Publication
(10) Pub. No.: US 2004/0091873 A1
(43) Pub. Date: May 13, 2004

(54) DNA polymerases with reduced base analog detection activity

(75) Inventors: Joseph A. Sorge, Wilson, WY (US); Holly Hogrefe, San Diego, CA (US); Madhushree Ghosh, San Diego, CA (US)

Correspondence Address: PALMER & DODGE, LLP, KATHLEEN M. WILLIAMS / STR, 111 HUNTINGTON AVENUE, BOSTON, MA, 02199, US

(73) Assignee: Stratagene

(21) Appl. No.: 10/298,680

(22) Filed: Nov. 18, 2002

Related U.S. Application Data
(63) Continuation-in-part of application No. 10/280,962, filed on Oct. 25, 2002.

(51) Int. Cl. C12Q 1/68; C07H 21/04; C12P 19/34; C12N 9/22; C12N 1/21

(52) U.S. Cl. 435/6; 435/69.1; 435/91.2; 435/199; 435/252.3; 435/320.1; 536/23.2

(57) ABSTRACT

The invention relates to the generation and characterization of archaeal DNA polymerase mutants with reduced base analog detection activity. The invention further provides for archaeal DNA polymerase mutants with reduced base analog detection activity containing additional mutations that modulate other DNA polymerase activities including DNA polymerization or 3′-5′ exonuclease activity. The invention also discloses methods and applications of DNA polymerases with reduced base analog detection activity.

### FIELD OF THE INVENTION

The invention relates to mutant archaeal DNA polymerases with reduced base analog detection activity.

### BACKGROUND

Unlike Taq, archaeal DNA polymerases (e.g., Pfu, Vent) possess a “read-ahead” function that detects uracil (dU) residues in the template strand and stalls synthesis (Greagg et al., 1999, PNAS USA, 96:9405). Uracil detection is thought to represent the first step in a pathway to repair DNA cytosine deamination (dCMP→dUMP) in archaea (Greagg et al, 1999, Supra). Stalling of DNA synthesis opposite uracil has significant implications for high-fidelity PCR amplification with archaeal DNA polymerases. Techniques requiring dUTP (e.g., dUTP/UDG decontamination methods, Longo et al. 1990, Gene, 93:125) or uracil-containing oligonucleotides can not be performed with proofreading DNA polymerases (Slupphaug et al. 1993, Anal. Biochem., 211:164; Sakaguchi et al. 1996, Biotechniques, 21:368). But more importantly, uracil stalling has been shown to compromise the performance of archaeal DNA polymerases under standard PCR conditions (Hogrefe et al. 2002, PNAS USA, 99:596).

During PCR amplification, a small amount of dCTP undergoes deamination to dUTP (% dUTP varies with cycling time), and is subsequently incorporated by archaeal DNA polymerases. Once incorporated, uracil-containing DNA inhibits archaeal DNA polymerases, limiting their efficiency. We found that adding a thermostable dUTPase (dUTP→dUMP+PPᵢ) to amplification reactions carried out with Pfu, KOD, Vent, and Deep Vent DNA polymerases significantly increases PCR product yields by preventing dUTP incorporation (Hogrefe et al. 2002, Supra). Moreover, the target-length capability of Pfu DNA polymerase is dramatically improved in the presence of dUTPase (from <2 kb to 14 kb), indicating that uracil poisoning severely limits long-range PCR due to the use of prolonged extension times (1-2 min per kb @ 72° C.) that promote dUTP formation.

In addition to dUTP incorporation, uracil may also arise as a result of cytosine deamination in template DNA. The extent to which cytosine deamination occurs during temperature cycling has not been determined; however, any uracil generated would presumably impair the PCR performance of archaeal DNA polymerases. Uracil arising from cytosine deamination in template DNA is unaffected by adding dUTPase, which only prevents incorporation of dUTP (created by dCTP deamination). Adding enzymes such as uracil DNA glycosylase (UGD), which excise uracil from the sugar backbone of DNA, or mismatch-specific UDGs (MUG), which additionally excise G:T mismatches, is one way to eliminate template uracil that impedes polymerization.

Alternatively, the problem of uracil stalling may be overcome by introducing mutations or deletions in archaeal DNA polymerases that reduce, or ideally, eliminate uracil detection, and therefore, allow synthesis to continue opposite incorporated uracil (non-mutagenic uracil) and deaminated cytosine (pro-mutagenic uracil). Such mutants would be expected to produce higher product yields and amplify longer targets compared to wild type archaeal DNA polymerases. Moreover, mutants that lack uracil detection should be compatible with dUTP/UNG decontamination methods employed in real-time Q-PCR. At present, only Taq and Taq-related enzymes can be used in clean-up methods based on dUTP incorporation.

There is therefore a need for thermostable DNA polymerases that can amplify DNA in the presence of dUTP without compromising proofreading or polymerization activity and efficiency.

Pavlov et al., 2002, Proc. Natl. Acad. Sci. USA, 99:13510-13515 and WO 01/92501 A1 describe polymerase chimeras comprising a domain that increases processivity and or increases salt resistance.

There is also a need in the art for thermostable DNA polymerases that can amplify DNA in the presence of dUTP without compromising proofreading or polymerization activity and efficiency, and wherein the thermostable DNA polymerase exhibits increased processivity and/or increased salt resistance.

### SUMMARY OF THE INVENTION

The invention relates to the construction and characterization of archacal Family B-type DNA polymerases mutants with reduced base analog detection activity that retain the essential PCR attributes of proofreading DNA polymerases (e.g., polymerase activity, 3′-5′ exonuclease activity, fidelity) and also improve the success rate of long-range amplification, e.g., higher yield, longer targets amplified.

The invention relates to mutant archaeal DNA polymerases, and in particular mutant Pfu DNA polymerases, with a reduced base analog detection activity, and comprising a mutation at position V93, that is a Valine substituted to Arginine, Glutamic acid, Lysine, Aspartic acid or Asparagine, or wherein the mutant archaeal DNA polymerase comprises a truncation, deletion or insertion, as defined herein.

The invention also provides for mutant archael DNA polymerases, including mutant Pfu DNA polymerases that further comprise a Glycine to Proline substitution at amino acid position 387 (G387P) that confers a reduced DNA polymerization phenotype to said mutant DNA polymerases or that further comprise an Aspartate to Alanine substitution at amino acid 141 (D141A) and a Glutamic acid to Alanine substitution at amino acid position 143 (D141A/E143A) that renders said mutant DNA polymerases 3′-5′ exonuclease deficient.

The invention also provides for a mutant archael DNA polymerase that is a chimera further comprising a polypeptide that increases the processivity of the polymerase and/or increases the salt resistance of the polymerase.

The invention also provides for isolated polynucleotide comprising a nucleotide sequence encoding these mutant archaeal DNA polymerases.

The invention also provides for a composition comprising a mutant archaeal DNA polymerase, including a Pfu DNA polymerase, having a reduced base analog detection activity, and comprising a mutation at position V93, wherein the mutation is a Valine substituted to Arginine, Glutamic acid, Lysine, Aspartic acid or Asparagine or wherein the mutant archaeal DNA polymerase comprises a truncation, deletion or insertion as defined herein. The invention also provides for a composition comprising a mutant archael DNA polymerase wherein the mutant archael DNA polymerase is a chimera comprising a polypeptide that increases processivity and or salt resistance. These compositions can further comprise Taq DNA polymerase or any DNA polymerase known in the art. In one embodiment, Taq DNA polymerase is at a 5 fold, 10 fold or 100 fold lower concentration than said mutant Pfu DNA polymerase. These compositions can also comprise a 3′ to 5′ exonuclease activity such as Pfu G387P or a Pfu chimera comprising a fusion Pfu polymerase wherein the Pfu polymerase is fused to a polypeptide that increases processivity and/or salt resistance. The invention also provides for compositions further comprising, a Pfu G387P/V93R or G387P/V93 E or G387P/V93 K or G387P/V93 D or G387P/V93N double mutant DNA polymerase, a Pfu V93R/D141A/E143A, Pfu V93E/D141A/E143A, Pfu V93K/D141A/E143A, Pfu V93D/D141A/E143A or Pfu V93N/D141A/E143A triple mutant DNA polymerase, Taq in combination with a Pfu G387P/V93R or G387P/V93 E or G387P/V93 K or G387P/V93 D or G387P/V93N double mutant, a Thermus DNA ligase or a FEN-1 nuclease, either alone or in combination with a PCR enhancing factor and/or an additive. The invention also provides for compositions comprising any of the single, double or triple mutant archael DNA polymerases described herein, any mutant archael DNA polymerases comprising an insertion, described herein, or any of the truncated, or deleted mutant archael DNA polymerases described herein, in combination with a polypeptide that increases processivity and or salt resistance, thereby forming a chimera, as defined herein. These chimeras can be provided in combination with a PCR enhancing factor and/or an additive.

The invention also provides for kits comprising a mutant archaeal DNA polymerase, including a Pfu DNA polymerase, having a reduced base analog detection activity, wherein the mutant archaeal DNA polymerase comprises a mutation at position V93 that is a Valine substituted to Arginine, Glutamic acid, Lysine, Aspartic acid or Asparagine wherein the mutant archaeal DNA polymerase comprises a truncation, deletion or insertion as defined herein, and packaging materials therefore. The kits of the invention may further comprise a PCR enhancing factor and/or an additive, Taq DNA polymerase, for example wherein said Taq DNA polymerase is at a 5 fold, 10 fold or 100 fold lower concentration than said mutant Pfu DNA polymerase, either alone or in combination with a PCR enhancing factor and/or an additive, or a Pfu G387P/V93R or G387P/V93 E or G387P/V93 K or G387P/V93 D or G387P/V93N double mutant DNA polymerase, a Pfu V93R/D141A/E143A, Pfu V93E/D141A/E143A, Pfu V93K/D141A/E143A, Pfu V93D/D141A/E143A or Pfu V93N/D141A/E143A triple mutant DNA polymerase, a Thermus DNA ligase or a FEN-1 nuclease, either alone or in combination with a PCR enhancing factor and/or an additive. The invention also provides for kits comprising any of the single, double or triple mutant archael DNA polymerases described herein, any mutant archael DNA polymerases comprising an insertion, described herein, or any of the truncated, or deleted mutant archael DNA polymerases described herein, in combination with a polypeptide that increases processivity and or salt resistance, thereby forming a chimera, as defined herein. These chimeras can be provided in combination with a PCR enhancing factor and/or an additive. The compositions of the invention can further comprise a chimera comprising a wild-type polymerase in combination with a polypeptide that increases processivity and/or salt resistance.

The invention also provides for a method for DNA synthesis comprising providing a mutant archaeal DNA polymerase of the invention; and contacting the enzyme with a nucleic acid template, wherein the enzyme permits DNA synthesis.

In one embodiment, DNA synthesis is performed in the presence of dUTP, for example as described in Example 3.

The invention also provides for a method for cloning of a DNA synthesis product comprising providing a mutant archaeal DNA polymerase of the invention, contacting the mutant archaeal DNA polymerase with a nucleic acid template, wherein the mutant archaeal DNA polymerase permits DNA synthesis to generate a synthesized DNA product; and inserting the synthesized DNA product into a cloning vector.

Any of the methods of amplification or cloning of the invention can further comprise a Thermus DNA ligase or a FEN-1 nuclease.

The invention also provides for a method for sequencing DNA comprising the step of providing a mutant archaeal DNA polymerase of the invention, generating chain terminated fragments from the DNA template to be sequenced with the mutant archaeal DNA polymerase in the presence of at least one chain terminating agent and one or more nucleotide triphosphates, and determining the sequence of the DNA from the sizes of said fragments. This method can be performed in the presence of Taq DNA polymerase, for example, wherein the Taq DNA polymerase is at a 5 fold, 10 fold or 100 fold lower concentration than said mutant Pfu DNA polymerase. Preferably sequencing is performed using an polymerase that is deficient in 3′ to 5′ exonuclease activity, for example D141A/E143A.

This method can also be carried out in the presence of a double or triple mutant DNA polymerase, as described herein, either alone or in combination with PCR enhancing factor and/or an additive.

Definitions

As used herein, “reduced base analog detection” refers to a DNA polymerase with a reduced ability to recognize a base analog, for example, uracil or inosine, present in a DNA template. In this context, mutant DNA polymerase with “reduced” base analog detection activity is a DNA polymerase mutant having a base analog detection activity which is lower than that of the wild-type enzyme, i.e., having less than 10% (e.g., less than 8%, 6%, 4%, 2% or less than 1%) of the base analog detection activity of that of the wild-type enzyme, base analog detection activity may be determined according to the assays similar to those described for the detection of DNA polymerases having a reduced uracil detection as described in Greagg et al. (1999) Proc. Natl. Acad. Sci. 96, 9045-9050 and Example 3. Alternatively, “reduced” base analog detection refers to a mutant DNA polymerase with a reduced ability to recognize a base analog, the “reduced” recognition of a base analog being evident by an increase in the amount of >10 Kb PCR of at least 10%, preferably 50%, more preferably 90%, most preferably 99% or more, as compared to a wild type DNA polymerase without a reduced base analog detection activity. The amount of a >10 Kb PCR product is measured either by spectorophotometer-absorbance assays of gel eluted >10 Kb PCR DNA product or by fluorometric analysis of >10 Kb PCR products in an ethidium bromide stained agarose electrophoresis gel using, for example, a Molecular Dynamics (MD) FluorImager™ (Amersham Biosciences, catalogue #63-0007-79).

As used herein, “reduced uracil detection” refers to a DNA polymerase with a reduced ability to recognize a uracil base present in a DNA template. In this context, mutant DNA polymerase with “reduced” uracil detection activity is a DNA polymerase mutant having a uracil detection activity which is lower than that of the wild-type enzyme, i.e., having less than 10% (e.g., less than 8%, 6%, 4%, 2% or less than 1%) of the uracil detection activity of that of the wild-type enzyme. Uracil detection activity may be determined according to the assays described in Greagg et al. (1999) Proc. Natl. Acad. Sci. 96, 9045-9050 and Example 3. Alternatively, “reduced” uracil detection refers to a mutant DNA polymerase with a reduced ability to recognize uracil, the “reduced” recognition of uracil being evident by an increase in the amount of >10 Kb PCR of at least 10%, preferably 50%, more preferably 90%, most preferably 99% or more, as compared to a wild type DNA polymerase without a reduced uracil detection activity. The amount of a >10 Kb PCR product is measured either by spectorophotometer-absorbance assays of gel eluted >10 Kb PCR DNA product or by fluorometric analysis of >10 Kb PCR products in an ethidium bromide stained agarose electrophoresis gel using, for example, a Molecular Dynamics (MD) FluorImager™ (Amersham Biosciences, catalogue #63-0007-79).

The invention contemplates mutant DNA polymerase that exhibits reduced base analog detection (for example, reduced detection of a particular base analog such as uracil or inosine or reduced detection of at least two base analogs).

As used herein, “base analogs” refer to bases that have undergone a chemical modification as a result of the elevated temperatures required for PCR reactions. In a preferred embodiment, “base analog” refers to uracil that is generated by deamination of cytosine. In another preferred embodiment, “base analog” refers to inosine that is generated by deamination of adenine.

As used herein, “synthesis” refers to any in vitro method for making new strand of polynucleotide or elongating existing polynucleotide (i.e., DNA or RNA) in a template dependent manner. Synthesis, according to the invention, includes amplification, which increases the number of copies of a polynucleotide template sequence with the use of a polymerase. Polynucleotide synthesis (e.g., amplification) results in the incorporation of nucleotides into a polynucleotide (i.e., a primer), thereby forming a new polynucleotide molecule complementary to the polynucleotide template. The formed polynucleotide molecule and its template can be used as templates to synthesize additional polynucleotide molecules.

“DNA synthesis”, according to the invention, includes, but is not limited to, PCR, the labelling of polynucleotide (i.e., for probes and oligonucleotide primers), polynucleotide sequencing.

As used herein, “polymerase” refers to an enzyme that catalyzes the polymerization of nucleotide (i.e., the polymerase activity). Generally, the enzyme will initiate synthesis at the 3′-end of the primer annealed to a polynucleotide template sequence, and will proceed toward the 5′ end of the template strand. “DNA polymerase” catalyzes the polymerization of deoxynucleotides. In a preferred embodiment, the “DNA polymerase” of the invention is an archaeal DNA polymerase. A “DNA polymerase” useful according to the invention includes, but is not limited to those included in the section of the present specification entitled “Polymerases”.

In a preferred embodiment, the DNA polymerase according to the invention is thermostable. In another preferred embodiment, the DNA polymerase according to the invention is Pfu DNA polymerase.

As used herein, “archaeal” DNA polymerase refers to DNA polymerases that belong to either the Family B/pol I-type group (e.g., Pfu, KOD, Pfx, Vent, Deep Vent, Tgo, Pwo) or the pol II group (e.g., Pyrococcus furiosus DP1/DP2 2-subunit DNA polymerase). In one embodiment, “archaeal” DNA polymerase refers to thermostable archaeal DNA polymerases (PCR-able) and include, but are not limited to, DNA polymerases isolated from Pyrococcus species (furiosus, species GB-D, woesii, abysii, horikoshii), Thermococcus species (kodakaraensis KOD1, litoralis, species 9 degrees North-7, species JDF-3, gorgonarius), Pyrodictium occultum, and Archaeoglobus fulgidus. It is estimated that suitable archaea would exhibit maximal growth temperatures of >80-85° C. or optimal growth temperatures of >70-80° C. Appropriate PCR enzymes from the archaeal pol I DNA polymerase group are commercially available, including Pfu (Stratagene), KOD (Toyobo), Pfx (Life Technologies, Inc.), Vent (New England BioLabs), Deep Vent (New England BioLabs), Tgo (Roche), and Pwo (Roche). Additional archaea related to those listed above are described in the following references: Archaea: A Laboratory Manual (Robb, F. T. and Place, A. R., eds.), Cold Spring Harbor Laboratory Press, Cold Spring Harbor, N.Y., 1995

As used herein, “mutant” polymerase refers to an archaeal DNA polymerase, as defined herein, comprising one or more mutations that alter one or more activities of the DNA polymerase, for example, DNA polymerization, 3′-5′ exonuclease activity or base analog detection activities. In one embodiment, the “mutant” polymerase of the invention refers to a DNA polymerase containing one or more mutations that reduce one or more base analog detection activities of the DNA polymerase. In a preferred embodiment, the “mutant” polymerase of the invention has a reduced uracil detection activity. In a preferred embodiment, the “mutant” polymerase of the invention has a reduced inosine detection activity. In another preferred embodiment, the “mutant” polymerase of the invention has a reduced uracil and inosine detection activity. A “mutant” polymerase as defined herein, includes a polymerase comprising one or more amino acid substitutions, one or more amino acid insertions, a truncation or an internal deletion.

A “mutant” polymerase as defined herein also includes a chimeric polymerase wherein any of the single, double or triple mutant archael DNA polymerases described herein, any mutant archael DNA polymerases comprising an insertion, described herein, or any of the truncated, or deleted mutant archael DNA polymerases described herein, occur in combination with a polypeptide that increases processivity and or salt resistance, thereby forming a chimera, as defined herein. A polypeptide that increases processivity and or salt resistance is described in WO 01/92501 A1 and Pavlov et al., 2002, Proc. Natl. Acad. Sci. USA, 99:13510-13515, herein incorporated by reference in their entirety.

A “chimera” as defined herein, is a fusion of a first amino acid sequence (protein) comprising a wild type or mutant archael DNA polymerase of the invention, joined to a second amino acid sequence defining a polypeptide that increases processivity and or increases salt resistance, wherein the first and second amino acids are not found in the same relationship in nature. A “chimera” according to the invention contains two or more amino acid sequences (for example a sequence encoding a wild type or mutant archael DNA polymerase and a polypeptide that increases processivity and/or salt resistance) from unrelated proteins, joined to form a new functional protein. A chimera of the invention may present a foreign polypeptide which is found (albeit in a different protein) in an organism which also expresses the first protein, or it may be an “interspecies”, “intergenic”, etc. fusion of protein structures expressed by different kinds of organisms. The invention encompasses chimeras wherein the polypeptide that increases processivity and/or salt resistance is joined N-terminally or C-terminally to a wild-type archael DNA polymerase or to any of the mutant archael DNA polymerases described herein.

As used herein, “polypeptide that increases processivity and/or salt resistance” refers to a domain that is a protein or a region of a protein or a protein complex, comprising a polypeptide sequence, or a plurality of peptide sequences wherein that region increases processivity, as defined herein, or increases salt resistance, as defined herein. A “polypeptide that increases processivity and/or salt resistance useful according to the invention includes but is not limited to any of the domains included in Pavlov et al., supra or WO 01/92501, for example Sso7d, Sac7d, HMF-like proteins, PCNA homologs, and helix-hairpin-helix domains, for example derived from Topoisomerase V.

As used herein, “joined” refers to any method known in the art for functionally connecting polypeptide domains, including without limitation recombinant fusion with or without intervening domains, intein-mediated fusion, non-covalent association, and covalent bonding, including disulfide bonding, hydrogen bonding, electrostatic bonding, and conformational bonding.

As used herein, “processivity” refers to the ability of a nucleic acid modifying enzyme, for example a polymerase, to remain attached to the template or substrate and perform multiple modification reactions. “Modification reactions” include but are not limited to polymerization, and exonucleolytic cleavage. “Processivity” also refers to the ability of a nucleic acid modifying enzyme, for example a polymerase, to modify relatively long (for example 0.5-1 kb, 1-5 kb or 5 kb or more) tracts of nucleotides. “Processivity” also refers to the ability of a nucleic acid modifying enzyme, for example a DNA polymerase, to perform a sequence of polymerization steps without intervening dissociation of the enzyme from the growing DNA chains. “Processivity” can depend on the nature of the polymerase, the sequence of a DNA template, and reaction conditions, for example, salt concentration, temperature or the presence of specific proteins.

As used herein, “increased processivity” refers to an increase of 5-10%, preferably 10-50%, more preferably 50-100% or more, as compared to a wild type or mutant archael DNA polymerase that lacks a polypeptide that increases processivity and/or salt resistance as defined herein. Processivity and increased processivity can be measured according the methods defined herein and in Pavlov et al., supra and WO 01/92501 A1. A polymerase with increased processivity that is a chimera comprising a polypeptide that increases processivity, as defined herein, is described in Pavlov et al. supra and WO 01/92501 A1.

As used herein, “increased salt resistance” refers to a polymerase that exhibits >50% activity at a salt concentration that is know to be greater than the maximum salt concentration at which the wild-type polymerase is active. The maximum salt concentration differs for each polymerase and is known in the art, or can be experimentally determined according to methods in the art. For example, Pfu is inhibited at 30 mM (in PCR) so a Pfu enzyme with increased salt resistance would have significant activity (>50%) at salt concentrations above 30 mM. A polymerase with increased salt resistance that is a chimera comprising a polypeptide that increases salt resistance, as defined herein, is described in Pavlov et al. supra and WO 01/92501 A1.

As used herein, a DNA polymerase with a “reduced DNA polymerization activity” is a DNA polymerase mutant comprising a DNA polymerization activity which is lower than that of the wild-type enzyme, e.g., comprising less than 10% DNA (e.g., less than 8%, 6%, 4%, 2% or less than 1%) polymerization activity of that of the wild-type enzyme. Methods used to generate characterize Pfu DNA polymerases with reduced DNA polymerization activity are disclosed in the pending U.S. patent application Ser. No. 10/035,091 (Hogrefe, et al.; filed: Dec. 21, 2001); the pending U.S. patent application Ser. No. 10/079,241 (Hogrefe, et al.; filed Feb. 20, 2002); the pending U.S. patent application Ser. No. 10/208,508 (Hogrefe et al.; filed Jul. 30, 2002); and the pending U.S. patent application Ser. No. 10/227,110 (Hogrefe et al.; filed Aug. 23, 2002), the contents of which are hereby incorporated in their entirety.

As used herein, “3′ to 5′ exonuclease deficient” or “3′ to 5′ exo-” refers to an enzyme that substantially lacks the ability to remove incorporated nucleotides from the 3′ end of a DNA polymer. DNA polymerase exonuclease activities, such as the 3′ to 5′ exonuclease activity exemplified by members of the Family B polymerases, can be lost through mutation, yielding an exonuclease-deficient polymerase. As used herein, a DNA polymerase that is deficient in 3′ to 5′ exonuclease activity substantially lacks 3′ to 5′ exonuclease activity. “Substantially lacks” encompasses a complete lack of activity, for example, 0.03%, 0.05%, 0.1%, 1%, 5%, 10%, 20% or even up to 50% of the exonuclease activity relative to the parental enzyme. Methods used to generate and characterize 3′-5′ exonuclease DNA polymerases including the D141A and E143A mutations as well as other mutations that reduce or eliminate 3′-5′ exonuclease activity are disclosed in the pending U.S. patent application Ser. No. 09/698,341 (Sorge et al; filed Oct. 27, 2000). Additional mutations that reduce or eliminate 3′ to 5′ exonuclease activity are known in the art and contemplated herein.

As used herein, “mutation” refers to a change introduced into a parental or wild type DNA sequence that changes the amino acid sequence encoded by the DNA, including, but not limited to, substitutions, insertions, deletions or truncations. The consequences of a mutation include, but are not limited to, the creation of a new character, property, function, or trait not found in the protein encoded by the parental DNA, including, but not limited to, N terminal truncation, C terminal truncation or chemical modification.

As used herein, “thermostable” refers to an enzyme which is stable and active at temperatures as great as preferably between about 90-100° C. and more preferably between about 70-98° C. to heat as compared, for example, to a non-thermostable form of an enzyme with a similar activity. For example, a thermostable nucleic acid polymerase derived from thermophilic organisms such as P. furiosus, M. jannaschii, A. fulgidus or P. horikoshii are more stable and active at elevated temperatures as compared to a nucleic acid polymerase from E. coli. A representative thermostable nucleic acid polymerase isolated from P. furiosus (Pfu) is described in Lundberg et al., 1991, Gene, 108:1-6. Additional representative temperature stable polymerases include, e.g., polymerases extracted from the thermophilic bacteria Thermus flavus, Thermus ruber, Thermus thermophilus, Bacillus stearothermophilus (which has a somewhat lower temperature optimum than the others listed), Thermus lacteus, Thermus rubens, Thermotoga maritima, or from thermophilic archaea Thermococcus litoralis, and Methanothermus fervidus.

Temperature stable polymerases are preferred in a thermocycling process wherein double stranded nucleic acids are denatured by exposure to a high temperature (about 95° C.) during the PCR cycle.

As used herein, the term “template DNA molecule” refers to that strand of a nucleic acid from which a complementary nucleic acid strand is synthesized by a DNA polymerase, for example, in a primer extension reaction.

As used herein, the term “template dependent manner” is intended to refer to a process that involves the template dependent extension of a primer molecule (e.g., DNA synthesis by DNA polymerase). The term “template dependent manner” refers to polynucleotide synthesis of RNA or DNA wherein the sequence of the newly synthesized strand of polynucleotide is dictated by the well-known rules of complementary base pairing (see, for example, Watson, J. D. et al., In: Molecular Biology of the Gene, 4th Ed., W. A. Benjamin, Inc., Menlo Park, Calif. (1987)).

The term “fidelity” as used herein refers to the accuracy of DNA polymerization by template-dependent DNA polymerase. The fidelity of a DNA polymerase is measured by the error rate (the frequency of incorporating an inaccurate nucleotide, i.e., a nucleotide that is not incorporated at a template-dependent manner). The accuracy or fidelity of DNA polymerization is maintained by both the polymerase activity and the 3′-5′ exonuclease activity of a DNA polymerase. The term “high fidelity” refers to an error rate of 5×10⁻⁶ per base pair or lower. The fidelity or error rate of a DNA polymerase may be measured using assays known to the art. For example, the error rates of DNA polymerase mutants can be tested using the lacI PCR fidelity assay described in Cline, J., Braman, J. C., and Hogrefe, H. H. (96) NAR 24:3546-3551. Briefly, a 1.9 kb fragment encoding the lacIOlacZα target gene is amplified from pPRIAZ plasmid DNA using 2.5 U DNA polymerase (i.e. amount of enzyme necessary to incorporate 25 nmoles of total dNTPs in 30 min. at 72° C.) in the appropriate PCR buffer. The lacI-containing PCR products are then cloned into lambda GT10 arms, and the percentage of lacI mutants (MF, mutation frequency) is determined in a color screening assay, as described (Lundberg, K. S., Shoemaker, D. D., Adams, M. W. W., Short, J. M., Sorge, J. A., and Mathur, E. J. (1991) Gene 180:1-8). Error rates are expressed as mutation frequency per bp per duplication (MF/bp/d), where bp is the number of detectable sites in the lacI gene sequence (349) and d is the number of effective target doublings. For each DNA polymerase mutant, at least two independent PCR amplifications are performed.

As used herein, an “amplified product” refers to the double strand polynucleotide population at the end of a PCR amplification reaction. The amplified product contains the original polynucleotide template and polynucleotide synthesized by DNA polymerase using the polynucleotide template during the PCR reaction.

As used herein, “polynucleotide template” or “target polynucleotide template” or “template” refers to a polynucleotide containing an amplified region. The “amplified region,” as used herein, is a region of a polynucleotide that is to be either synthesized by polymerase chain reaction (PCR). For example, an amplified region of a polynucleotide template resides between two sequences to which two PCR primers are complementary to.

As used herein, the term “primer” refers to a single stranded DNA or RNA molecule that can hybridize to a polynucleotide template and prime enzymatic synthesis of a second polynucleotide strand. A primer useful according to the invention is between 10 to 100 nucleotides in length, preferably 17-50 nucleotides in length and more preferably 17-45 nucleotides in length.

“Complementary” refers to the broad concept of sequence complementarity between regions of two polynucleotide strands or between two nucleotides through base-pairing. It is known that an adenine nucleotide is capable of forming specific hydrogen bonds (“base pairing”) with a nucleotide which is thymine or uracil. Similarly, it is known that a cytosine nucleotide is capable of base pairing with a guanine nucleotide.

The term “wild-type” refers to a gene or gene product which has the characteristics of that gene or gene product when isolated from a naturally occurring source. In contrast, the term “modified” or “mutant” refers to a gene or gene product which displays altered characteristics when compared to the wild-type gene or gene product. For example, a mutant DNA polymerase in the present invention is a DNA polymerase which exhibits a reduced uracil detection activity.

As used herein “FEN-1 nuclease” refers to thermostable FEN-1 endonucleases useful according to the invention and include, but are not limited to, FEN-1 endonuclease purified from the “hyperthermophiles”, e.g., from M. jannaschii, P. furiosus and P. woesei. See U.S. Pat. No. 5,843,669, hereby incorporated by reference.

According to the methods of the present invention, the addition of FEN-1 in the amplification reaction dramatically increases the efficiency of the multi-site mutagenesis. 400 ng to 4000 ng of FEN-1 may be used in each amplification reaction. Preferably 400-1000 ng, more preferably, 400-600 ng of FEN-1 is used in the amplification reaction. In a preferred embodiment of the invention, 400 ng FEN-1 is used.

As used herein, “Thermus DNA ligase” refers to a thermostable DNA ligase that is used in the multi-site mutagenesis amplification reaction to ligate the mutant fragments synthesized by extending each mutagenic primer so to form a circular mutant strand. Tth and Taq DNA ligase require NAD as a cofactor.

Preferably, 1-20 U DNA ligase is used in each amplification reaction, more preferably, 2-15 U DNA ligase is used in each amplification reaction.

In a preferred embodiment, 15 U Taq DNA ligase is used in an amplification reaction. Taq DNA ligase cofactor NAD is used at a concentration of 0-1 mM, preferably between 0.02-0.2 mM, more preferably at 0.1 mM.

As used herein, a “PCR enhancing factor” or a “Polymerase Enhancing Factor” (PEF) refers to a complex or protein possessing polynucleotide polymerase enhancing activity including, but not limited to, PCNA, RFC, helicases etc (Hogrefe et al., 1997, Strategies 10:93-96; and U.S. Pat. No. 6,183,997, both of which are hereby incorporated by reference).

The invention also contemplates mutant archael DNA polymerases in combination with accessory factors, for example as described in U.S. Pat. No. 6,333,158, and WO 01/09347 A2, hereby incorporated by reference in its entirety.

As used herein, a mutant archaeal or Pfu DNA polymerase comprising a truncation refers to a truncated DNA polymerase with reduced base analog detection activity, preferably reduced uracil detection activity, that is 3′-5′ exonuclease deficient and comprises an N terminal truncation from amino acid 1-4, preferably amino acid 1-93 or most preferably amino acid 1-337.

In one embodiment, a mutant archaeal or Pfu DNA polymerase comprising a truncation refers to a truncated DNA polymerase with reduced base analog detection activity, preferably reduced uracil detection activity, that is 3′-5′ exonuclease deficient and comprises an N terminal truncation wherein at least the first N terminal amino acid is removed and wherein no more than the first 337 N terminal amino acids are removed or wherein at least the first 1-7 N-terminal amino acids are removed and wherein no more than the first 337 N-terminal amino acids are removed.

In one embodiment, a mutant archaeal or Pfu DNA polymerase comprising a truncation is a truncated DNA polymerase with 3′-5′ exonuclease activity and reduced base analog detection activity, preferably reduced uracil detection activity, that comprises an N terminal truncation from amino acid 1-7, preferably 1-38, more preferably 1-93, more preferably 1-116 or most preferably amino acid 1 to amino acid 136.

In another embodiment, a mutant archaeal or Pfu DNA polymerase comprising a truncation is a truncated DNA polymerase with 3′-5′ exonuclease activity and reduced base analog detection activity, preferably reduced uracil detection activity, that comprises an N terminal truncation wherein at least the first 1 to 7 N-terminal amino acid is/are removed and wherein no more than the first 136 N-terminal amino acids are removed.

As used herein, a mutant archaeal or Pfu DNA polymerase with an internal deletion refers to a mutant archaeal or Pfu DNA polymerase with reduced base analog detection activity, preferably reduced uracil detection activity, that contains an internal deletion of 1 amino acid, 2-4 amino acids, 5-10 amino acids, 10-25 amino acids, 25-50 amino acids, 50-75 amino acids, 75-100 amino acids, or most preferably 136 amino acids within the first N terminal 136 amino acids of the mutant archaeal or Pfu DNA polymerase.

In another embodiment, a mutant archaeal or Pfu DNA polymerase with an internal deletion refers to a mutant archaeal or Pfu DNA polymerase with reduced base analog detection activity, preferably reduced uracil detection activity, that is 3′-5′ exonuclease deficient and comprises an internal deletion of 1 amino acid, 1-5 amino acids, 5-10 amino acids, 10-25 amino acids, 25-50 amino acids, 50-100 amino acids, 100-150 amino acids, 150-200 amino acids, preferably 200-250 amino acids, preferably 250-300 amino acids, or most preferably 337 amino acids within the first N terminal 337 amino acids of the mutant archacal or Pfu DNA polymerase.

In another embodiment, the mutant archaeal or Pfu DNA polymerase with an internal deletion is a DNA polymerase with 3′-5′ exonuclease activity and reduced base analog detection activity, preferably reduced uracil detection activity, and comprises an internal deletion of one or more amino acids in the regions of amino acids 6-8, amino acids 36-38, amino acids 90-97 and amino acids 111-116.

In another embodiment, the mutant archaeal or Pfu DNA polymerase with an internal deletion is a DNA polymerase with reduced base analog detection activity, preferably reduced uracil detection activity, that is 3′-5′ exonuclease deficient and comprises an internal deletion of one or more amino acids in the regions of amino acids 6-8, amino acids 36-38, amino acids 90-97 and amino acids 111-116.

### BRIEF DESCRIPTION OF THE DRAWINGS

FIG. 1: Oligonucleotide Primers for QuikChange Mutagenesis (SEQ ID Nos: 6-14)

FIG. 2: (a) dUTP incorporation of V93E and V93R mutants compared to wild type Pfu DNA polymerase.

(b) PCR Amplification of Pfu V93R mutant extract in the presence of 100% dUTP.

FIG. 3: Protein concentration, unit concentration, and specific activity of the purified Pfu V93R and V93E mutants.

FIG. 4: Comparison of the efficacy of PCR amplification of Pfu DNA polymerase mutants and wt enzyme in the presence of different TTP:dUTP concentration ratios.

FIG. 5: Comparison of the efficacy of “long” PCR amplification of Pfu DNA polymerase mutants and wt enzyme.

FIG. 6: 6A. DNA sequence of mutant archeael DNA polymerases

6B. Amino acid sequence of mutant archeael DNA polymerases

6C. DNA and Amino acid sequence of mutant Tgo DNA polymerase

FIG. 7: DNA and Amino acid sequence of wild type Pfu DNA polymerase

FIG. 8: dUTP incorporation of Pfu mutants compared to wild type Pfu DNA polymerase

8A. dUTP incorporation of Pfu mutants V93W, V93Y, V93M, V93K and V93R compared to wild type Pfu DNA polymerase

8B. dUTP incorporation of the Pfu V93D and V93R mutants compared to wild type Pfu DNA polymerase.

8C. dUTP incorporation of the Pfu V93N and V93G mutant compared to wild type Pfu DNA polymerase

FIG. 9: DNA polymerase activity of N-terminal Pfu DNA polymerase truncation mutants.

### DETAILED DESCRIPTION

Base deamination and other base modifications greatly increase as a consequence of PCR reaction conditions, for example, elevated temperature. This results in the progressive accumulation of base analogs (for example uracil or inosine) in the PCR reaction that ultimately inhibit archaeal proofreading DNA polymerases, such as Pfu, Vent and Deep Vent DNA polymerases, severely limiting their efficiency.

The present invention provides a remedy to the problem of base analog contamination of PCR reactions by disclosing methods for the isolation and characterization of archaeal DNA polymerases with reduced base analog detection activities.

The mutant archael DNA polymerases of the invention may provide for the use of fewer units of polymerase, may allow assays to be done using shorter extension times and/or may provide greater success in achieving higher yields and or longer products.

Archaeal DNA Polymerases

There are 2 different classes of DNA polymerases which have been identified in archaea: 1. Family B/pol I type (homologs of Pfu from Pyrococcus furiosus) and 2. pol II type (homologs of P. furiosus DP1/DP2 2-subunit polymerase). DNA polymerases from both classes have been shown to naturally lack an associated 5′ to 3′ exonuclease activity and to possess 3′ to 5′ exonuclease (proofreading) activity. Suitable DNA polymerases (pol I or pol II) can be derived from archaea with optimal growth temperatures that are similar to the desired assay temperatures.

Thermostable archaeal DNA polymerases isolated from Pyrococcus species (furiosus, species GB-D, woesii, abysii, horikoshii), Thermococcus species (kodakaraensis KOD1, litoralis, species 9 degrees North-7, species JDF-3, gorgonarius), Pyrodictium occultum, and Archaeoglobus fulgidus. It is estimated that suitable archaea would exhibit maximal growth temperatures of >80-85° C. or optimal growth temperatures of >70-80° C. Appropriate PCR enzymes from the archaeal pol I DNA polymerase group are commercially available, including Pfu (Stratagene), KOD (Toyobo), Pfx (Life Technologies, Inc.), Vent (New England BioLabs), Deep Vent (New England BioLabs), Tgo (Roche), and Pwo (Roche).

Additional archaea DNA polymerases related to those listed above are described in the following references: Archaea: A Laboratory Manual (Robb, F. T. and Place, A. R., eds.), Cold Spring Harbor Laboratory Press, Cold Spring Harbor, N.Y., 1995 and Thermophilic Bacteria (Kristjansson, J. K., ed.) CRC Press, Inc., Boca Raton, Fla., 1992.

The invention therefore provides for thermostable archaeal DNA polymerases of either Family B/pol I type or pol II type with a reduced base analog detection activity.

TABLE 1
ACCESSION INFORMATION FOR CLONED FAMILY B
POLYMERASES

Vent Thermococcus litoralis
ACCESSION AAA72101
PID g348689
VERSION AAA72101.1 GI:348689
DBSOURCE locus THCVDPE accession M74198.1
THEST THERMOCOCCUS SP. (STRAIN TY)
ACCESSION O33845
PID g3913524
VERSION O33845 GI:3913524
DBSOURCE swissprot: locus DPOL_THEST, accession O33845
Pab Pyrococcus abyssi
ACCESSION P77916
PID g3913529
VERSION P77916 GI:3913529
DBSOURCE swissprot: locus DPOL_PYRAB, accession P77916
PYRHO Pyrococcus horikoshii
ACCESSION O59610
PID g3913526
VERSION O59610 GI:3913526
DBSOURCE swissprot: locus DPOL_PYRHO, accession O59610
PYRSE PYROCOCCUS SP. (STRAIN GE23)
ACCESSION P77932
PID g3913530
VERSION P77932 GI:3913530
DBSOURCE swissprot: locus DPOL_PYRSE, accession P77932
Deep Vent Pyrococcus sp.
ACCESSION AAA67131
PID g436495
VERSION AAA67131.1 GI:436495
DBSOURCE locus PSU00707 accession U00707.1
Pfu Pyrococcus furiosus
ACCESSION P80061
PID g399403
VERSION P80061 GI:399403
DBSOURCE swissprot: locus DPOL_PYRFU, accession P80061
JDF-3 Thermococcus sp.
Unpublished
Baross gi|2097756|pat|US|5602011|12 Sequence 12 from patent US
5602011 9degN THERMOCOCCUS SP. (STRAIN 9ON-7).
ACCESSION Q56366
PID g3913540
VERSION Q56366 GI:3913540
DBSOURCE swissprot: locus DPOL_THES9, accession Q56366
KOD Pyrococcus sp.
ACCESSION BAA06142
PID g1620911
VERSION BAA06142.1 GI:1620911
DBSOURCE locus PYWKODPOL accession D29671.1
Tgo Thermococcus gorgonarius.
ACCESSION 4699806
PID g4699806
VERSION GI:4699806
DBSOURCE pdb: chain 65, release Feb. 23, 1999
THEFM Thermococcus fumicolans
ACCESSION P74918
PID g3913528
VERSION P74918 GI:3913528
DBSOURCE swissprot: locus DPOL_THEFM, accession P74918
METTH Methanobacterium thermoautotrophicum
ACCESSION O27276
PID g3913522
VERSION O27276 GI:3913522
DBSOURCE swissprot: locus DPOL_METTH, accession
O27276
Metja Methanococcus jannaschii
ACCESSION Q58295
PID g3915679
VERSION Q58295 GI:3915679
DBSOURCE swissprot: locus DPOL_METJA, accession Q58295
POC Pyrodictium occultum
ACCESSION B56277
PID g1363344
VERSION B56277 GI:1363344
DBSOURCE pir: locus B56277
Apel Aeropyrum pernix
ACCESSION BAA81109
PID g5105797
VERSION BAA81109.1 GI:5105797
DBSOURCE locus AP000063 accession AP000063.1
ARCFU Archaeoglobus fulgidus
ACCESSION O29753
PID g3122019
VERSION O29753 GI:3122019
DBSOURCE swissprot: locus DPOL_ARCFU, accession O29753
Desulfurococcus sp. Tok.
ACCESSION 6435708
PID g64357089
VERSION GT:6435708
DBSOURCE pdb. chain 65, release Jun. 2, 1999

II. Preparing Mutant DNA Polymerase with Reduced Base Analog Detection Activity

Cloned wild-type DNA polymerases may be modified to generate forms exhibiting reduced base analog detection activity by a number of methods. These include the methods described below and other methods known in the art. Any proofreading archaeal DNA polymerase can be used to prepare for DNA polymerase with reduced base analog detection activity in the invention.

Genetic Modifications—Mutagenesis

Direct comparison of DNA polymerases from diverse organisms indicates that the domain structure of these enzymes is highly conserved and in many instances, it is possible to assign a particular function to a well-defined domain of the enzyme. For example, the six most conserved C-terminal regions, spanning approximately 340 amino acids, are located in the same linear arrangement and contain highly conserved motifs that form the metal and dNTP binding sites and the cleft for holding the DNA template and are therefore essential for the polymerization function. In another example, the three amino acid regions containing the critical residues in the E. coli DNA polymerase I involved in metal binding, single-stranded DNA binding, and catalysis of the 3′->5′ exonuclease reaction are located in the amino-terminal half and in the same linear arrangement in several prokaryotic and eukaryotic DNA polymerases. The location of these conserved regions provides a useful model to direct genetic modifications for preparing DNA polymerase with reduced base analog detection activity whilst conserving essential functions e.g. DNA polymerization and proofreading activity.

The preferred method of preparing a DNA polymerase with reduced base analog detection activity is by genetic modification (e.g., by modifying the DNA sequence of a wild-type DNA polymerase). A number of methods are known in the art that permit the random as well as targeted mutation of DNA sequences (see for example, Ausubel et. al. Short Protocols in Molecular Biology (1995)₃rd Ed. John Wiley & Sons, Inc.). In addition, there are a number of commercially available kits for site-directed mutagenesis, including both conventional and PCR-based methods. Examples include the EXSITE™ PCR-Based Site-directed Mutagenesis Kit available from Stratagene (Catalog No. 200502) and the QUIKCHANGE™ Site-directed mutagenesis Kit from Stratagene (Catalog No. 200518), and the CHAMELEON® double-stranded Site-directed mutagenesis kit, also from Stratagene (Catalog No. 200509).

In addition DNA polymerases with reduced base analog detection activity may be generated by insertional mutation or truncation (N-terminal, internal or C-terminal) according to methodology known to a person skilled in the art.

Older methods of site-directed mutagenesis known in the art rely on sub-cloning of the sequence to be mutated into a vector, such as an M13 bacteriophage vector, that allows the isolation of single-stranded DNA template. In these methods, one anneals a mutagenic primer (i.e., a primer capable of annealing to the site to be mutated but bearing one or mismatched nucleotides at the site to be mutated) to the single-stranded template and then polymerizes the complement of the template starting from the 3′ end of the mutagenic primer. The resulting duplexes are then transformed into host bacteria and plaques are screened for the desired mutation.

More recently, site-directed mutagenesis has employed PCR methodologies, which have the advantage of not requiring a single-stranded template. In addition, methods have been developed that do not require sub-cloning. Several issues must be considered when PCR-based site-directed mutagenesis is performed. First, in these methods it is desirable to reduce the number of PCR cycles to prevent expansion of undesired mutations introduced by the polymerase. Second, a selection must be employed in order to reduce the number of non-mutated parental molecules persisting in the reaction. Third, an extended-length PCR method is preferred in order to allow the use of a single PCR primer set. And fourth, because of the non-template-dependent terminal extension activity of some thermostable polymerases it is often necessary to incorporate an end-polishing step into the procedure prior to blunt-end ligation of the PCR-generated mutant product.

The protocol described below accommodates these considerations through the following steps. First, the template concentration used is approximately 1000-fold higher than that used in conventional PCR reactions, allowing a reduction in the number of cycles from 25-30 down to 5-10 without dramatically reducing product yield. Second, the restriction endonuclease Dpn I (recognition target sequence: 5-Gm6ATC-3, where the A residue is methylated) is used to select against parental DNA, since most common strains of E. coli Dam methylate their DNA at the sequence 5-GATC-3. Third, Taq Extender is used in the PCR mix in order to increase the proportion of long (i.e., full plasmid length) PCR products. Finally, Pfu DNA polymerase is used to polish the ends of the PCR product prior to intramolecular ligation using T4 DNA ligase.

A non-limiting example for the isolation of mutant archaeal DNA polymerases exhibiting reduced uracil detection activity is described in detail as follows:

Plasmid template DNA (approximately 0.5 pmole) is added to a PCR cocktail containing: 1× mutagenesis buffer (20 mM Tris HCl, pH 7.5; 8 mM MgCl₂; 40 μg/ml BSA); 12-20 pmole of each primer (one of skill in the art may design a mutagenic primer as necessary, giving consideration to those factors such as base composition, primer length and intended buffer salt concentrations that affect the annealing characteristics of oligonucleotide primers; one primer must contain the desired mutation, and one (the same or the other) must contain a 5′ phosphate to facilitate later ligation), 250 μM each dNTP, 2.5 U Taq DNA polymerase, and 2.5 U of Taq Extender (Available from Stratagene; See Nielson et al. (1994) Strategies 7: 27, and U.S. Pat. No. 5,556,772). Primers can be prepared using the triester method of Matteucci et al., 1981, J. Am. Chem. Soc. 103:3185-3191, incorporated herein by reference. Alternatively automated synthesis may be preferred, for example, on a Biosearch 8700 DNA Synthesizer using cyanoethyl phosphoramidite chemistry.

The PCR cycling is performed as follows: 1 cycle of 4 min at 94° C., 2 min at 50° C. and 2 min at 72° C.; followed by 5-10 cycles of 1 min at 94° C., 2 min at 54° C. and 1 min at 72° C. The parental template DNA and the linear, PCR-generated DNA incorporating the mutagenic primer are treated with DpnI (10 U) and Pfu DNA polymerase (2.5 U). This results in the DpnI digestion of the in vivo methylated parental template and hybrid DNA and the removal, by Pfu DNA polymerase, of the non-template-directed Taq DNA polymerase-extended base(s) on the linear PCR product. The reaction is incubated at 37° C. for 30 min and then transferred to 72° C. for an additional 30 min. Mutagenesis buffer (115 ul of 1×) containing 0.5 mM ATP is added to the DpnI-digested, Pfu DNA polymerase-polished PCR products. The solution is mixed and 10 ul are removed to a new microfuge tube and T4 DNA ligase (2-4 U) is added. The ligation is incubated for greater than 60 min at 37° C. Finally, the treated solution is transformed into competent E. coli according to standard methods.

Methods of random mutagenesis, which will result in a panel of mutants bearing one or more randomly situated mutations, exist in the art. Such a panel of mutants may then be screened for those exhibiting reduced uracil detection activity relative to the wild-type polymerase (e.g., by measuring the incorporation of 10 nmoles of dNTPs into polymeric form in 30 minutes in the presence of 200 μM dUTP and at the optimal temperature for a given DNA polymerase). An example of a method for random mutagenesis is the so-called “error-prone PCR method”. As the name implies, the method amplifies a given sequence under conditions in which the DNA polymerase does not support high fidelity incorporation. The conditions encouraging error-prone incorporation for different DNA polymerases vary, however one skilled in the art may determine such conditions for a given enzyme. A key variable for many DNA polymerases in the fidelity of amplification is, for example, the type and concentration of divalent metal ion in the buffer. The use of manganese ion and/or variation of the magnesium or manganese ion concentration may therefore be applied to influence the error rate of the polymerase.

Genes for desired mutant DNA polymerases generated by mutagenesis may be sequenced to identify the sites and number of mutations. For those mutants comprising more than one mutation, the effect of a given mutation may be evaluated by introduction of the identified mutation to the wild-type gene by site-directed mutagenesis in isolation from the other mutations borne by the particular mutant. Screening assays of the single mutant thus produced will then allow the determination of the effect of that mutation alone.

In a preferred embodiment, the enzyme with reduced uracil detection activity is derived from archaeal DNA polymerase containing one or more mutations.

In a preferred embodiment, the enzyme with reduced uracil detection activity is derived from Pfu DNA polymerase.

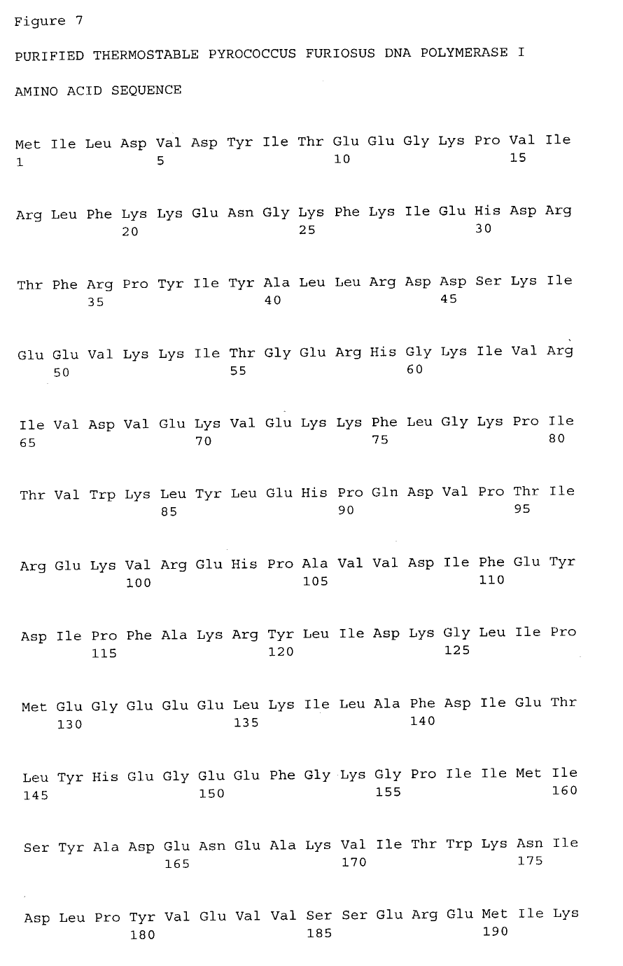

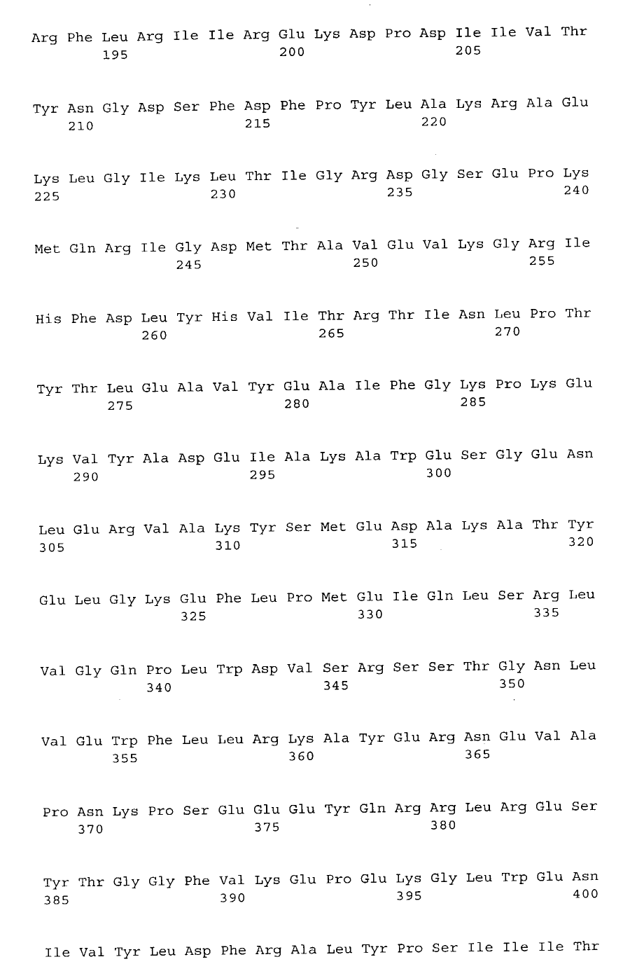

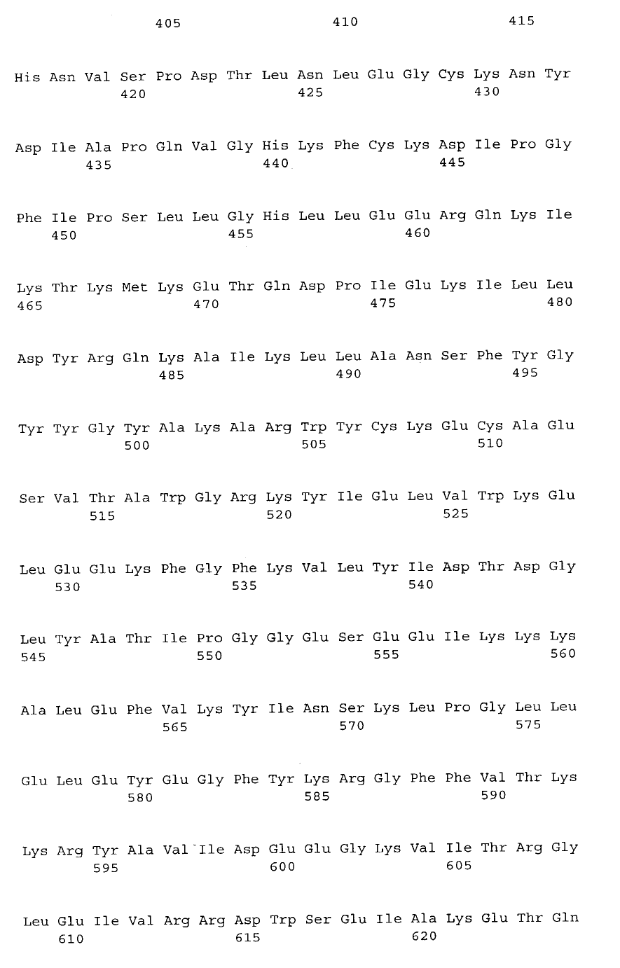

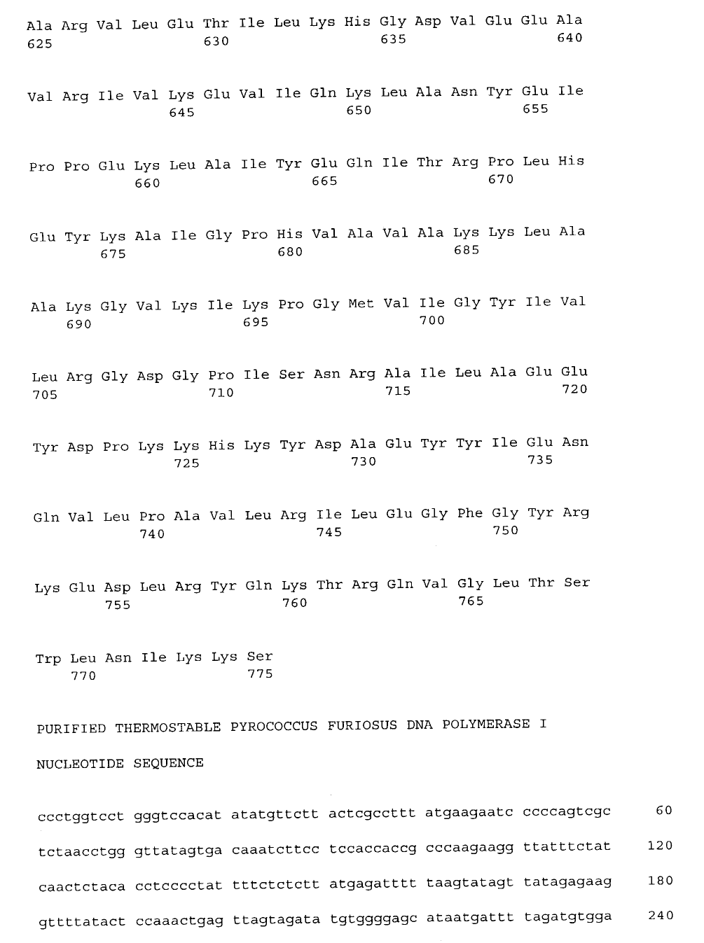

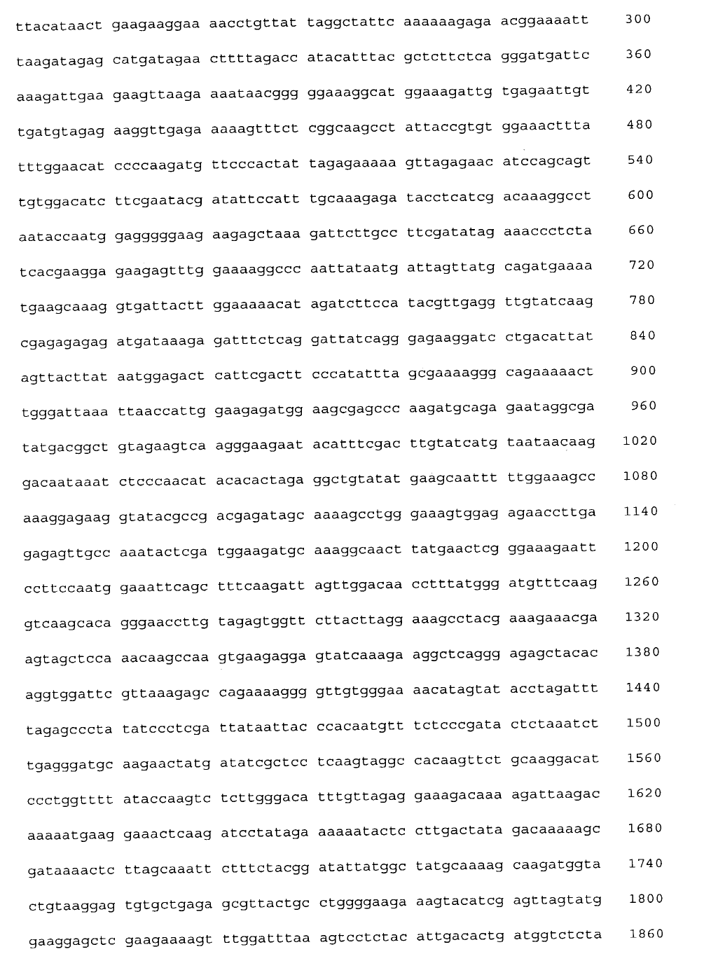

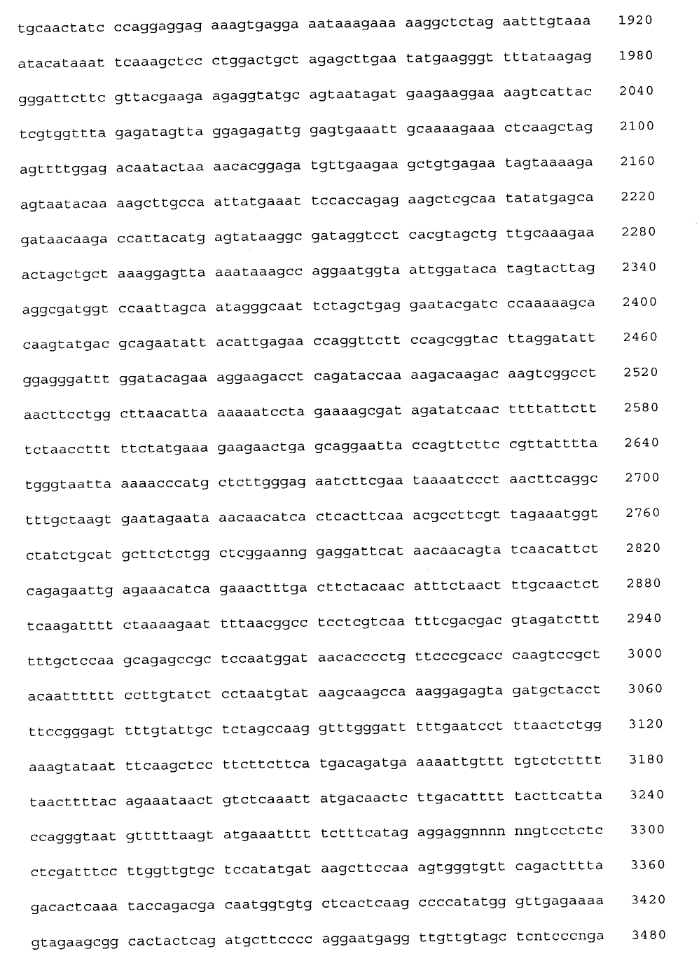

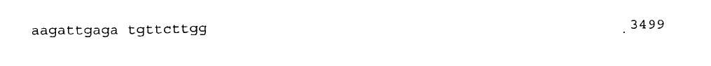

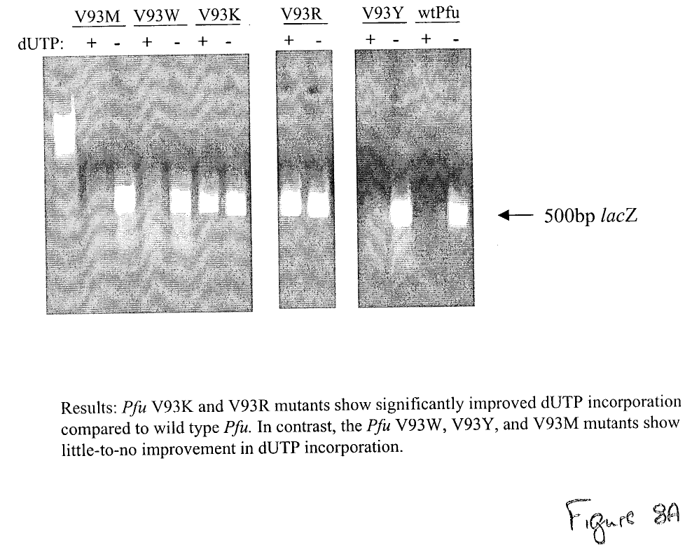

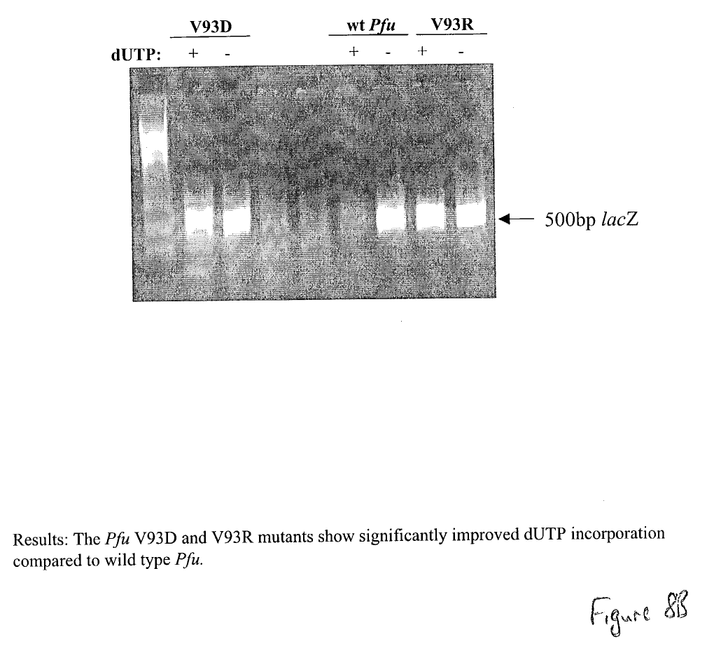

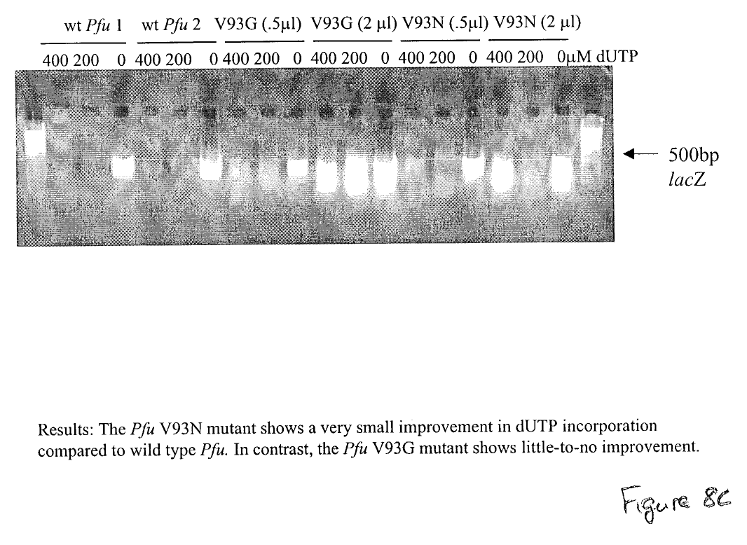

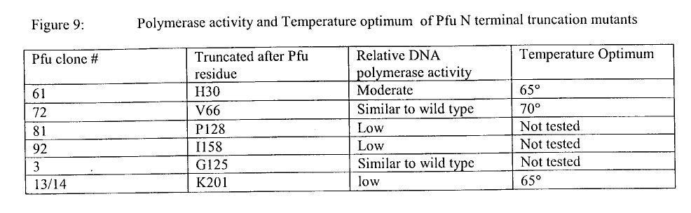

The amino acid and DNA coding sequence of a wild-type Pfu DNA polymerase are shown in FIG. 7 (Genbank Accession # P80061). A detailed description of the structure and function of Pfu DNA polymerase can be found, among other places in U.S. Pat. Nos. 5,948,663; 5,866,395; 5,545,552; 5,556,772, all of which thereby incorporated by references. A non-limiting detailed procedure for preparing Pfu DNA polymerase with reduced uracil detection activity is provided in Example 1.

A person of average skill in the art having the benefit of this disclosure will recognize that polymerases with reduced uracil detection activity derived from other exo⁺ DNA polymerases including Vent DNA polymerase, JDF-3 DNA polymerase, Tgo DNA polymerase, and the like may be suitably used in the subject compositions.

The enzyme of the subject composition may comprise DNA polymerases that have not yet been isolated.

In preferred embodiments of the invention, the mutant Pfu DNA polymerase harbors an amino acid substitution at amino acid position, V93. In a preferred embodiment, the mutant Pfu DNA polymerase of the invention contains a Valine to Arginine, Valine to Glutamic acid, Valine to Lysine, Valine to Aspartic Acid or Valine to Asparagine substitution at amino acid position 93.

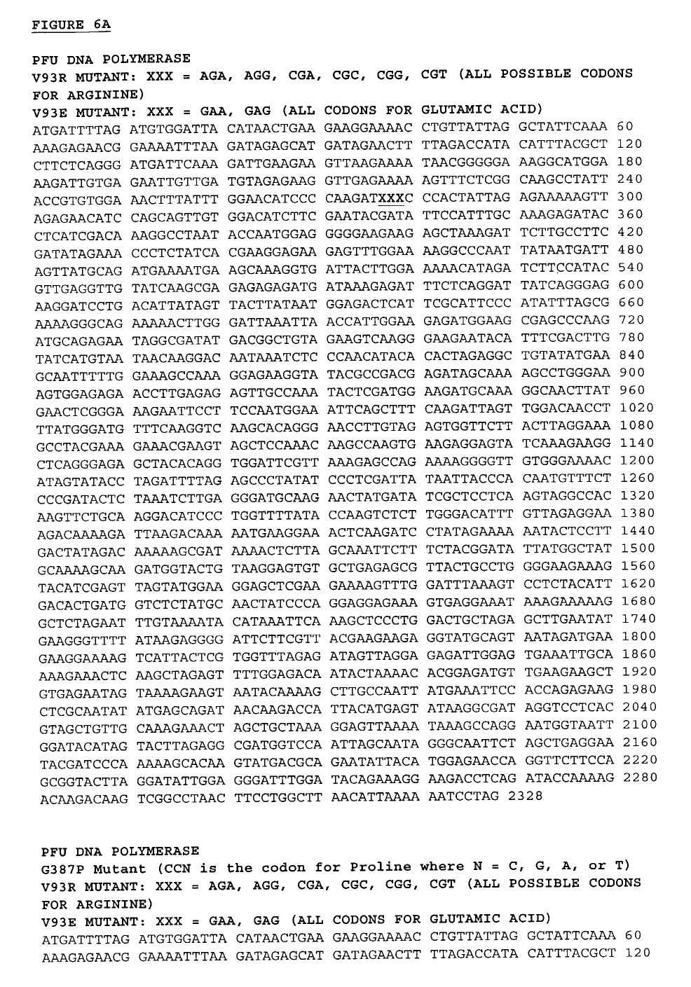

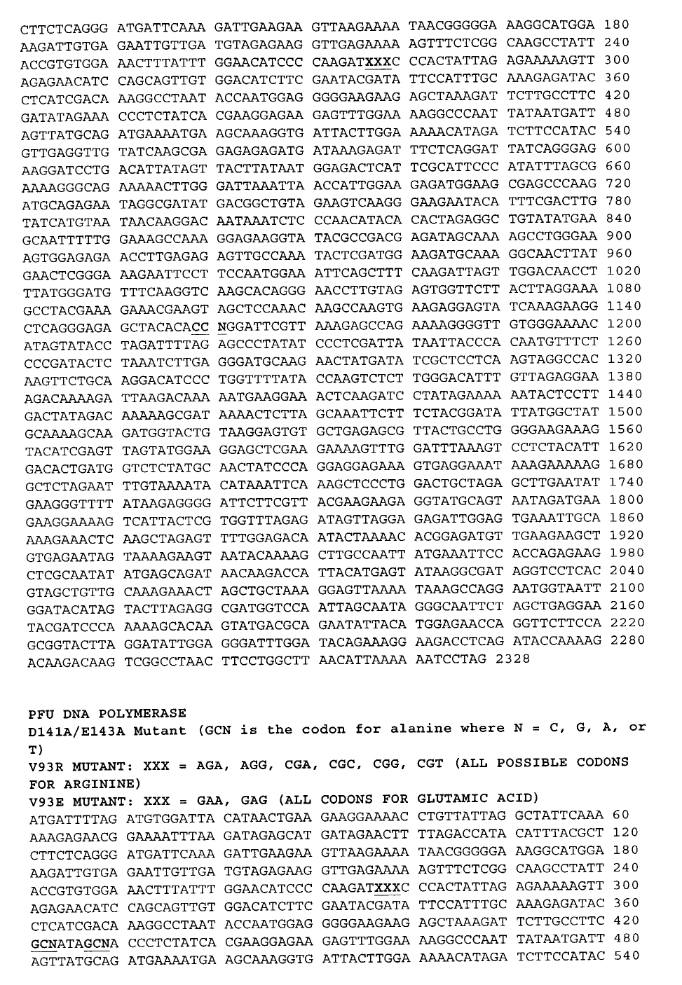

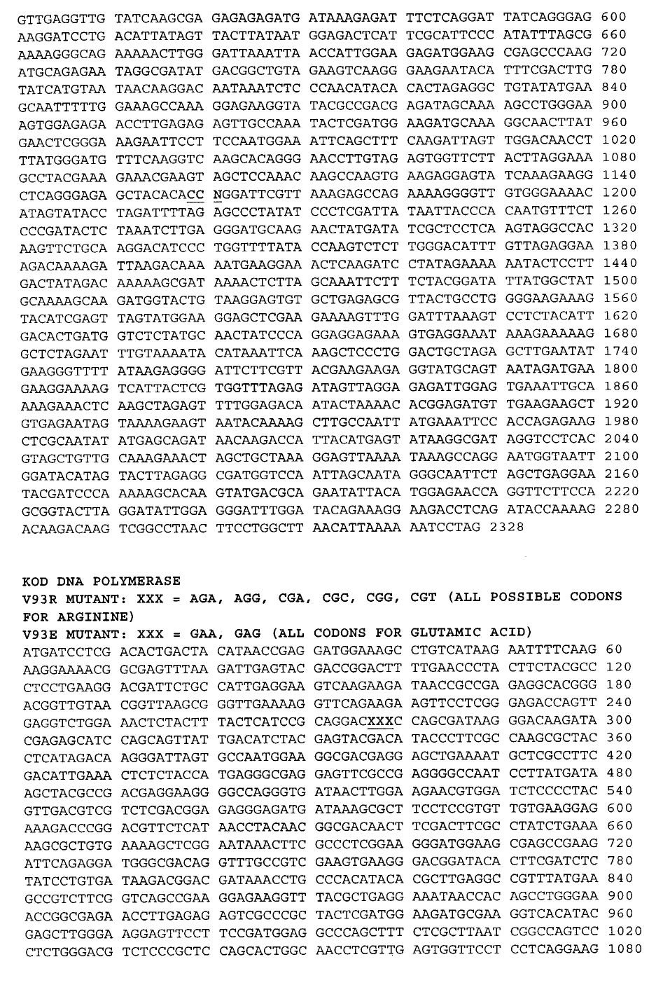

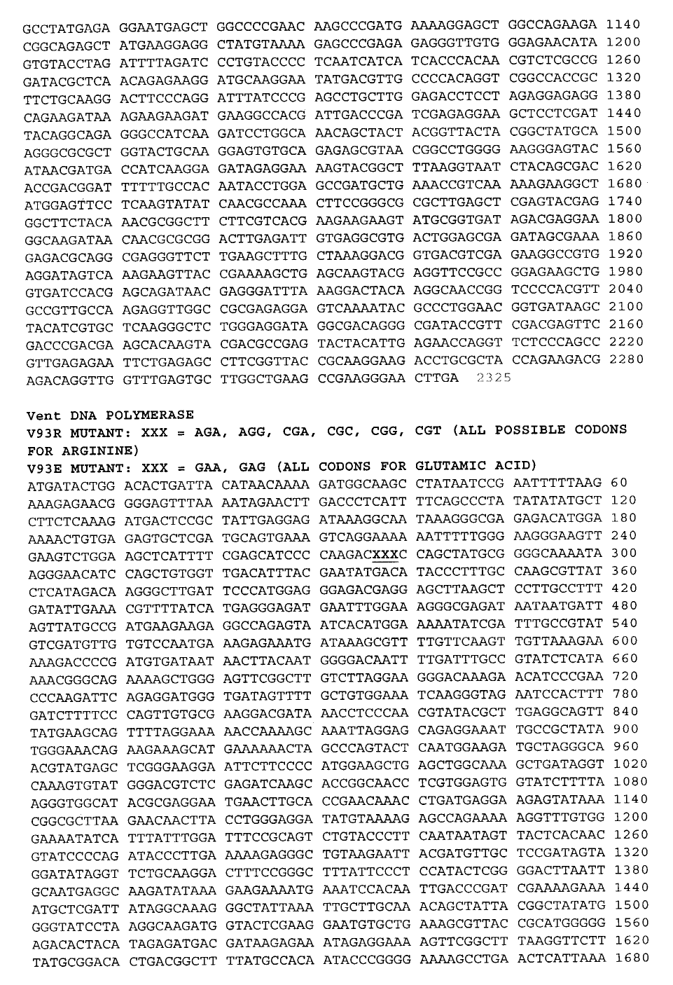

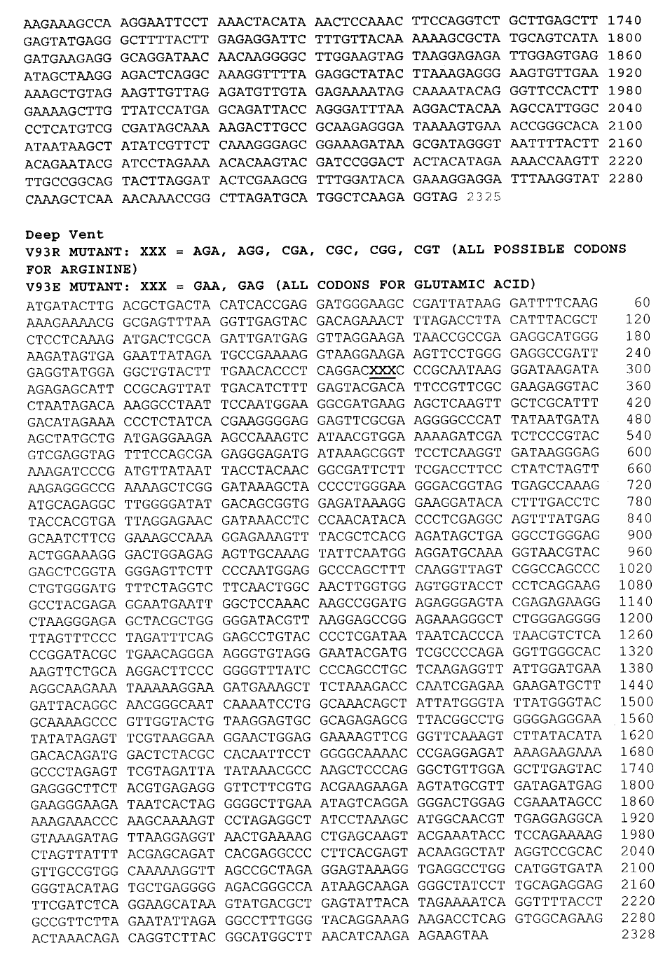

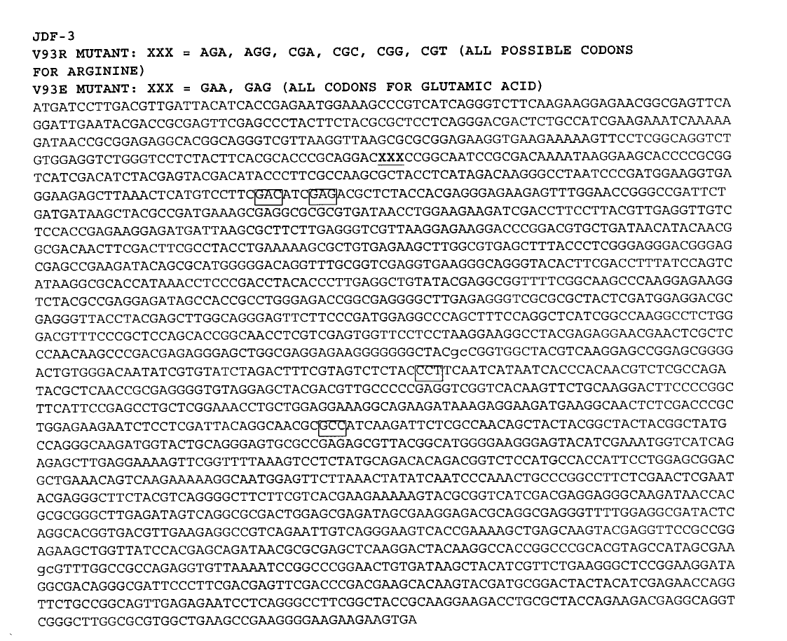

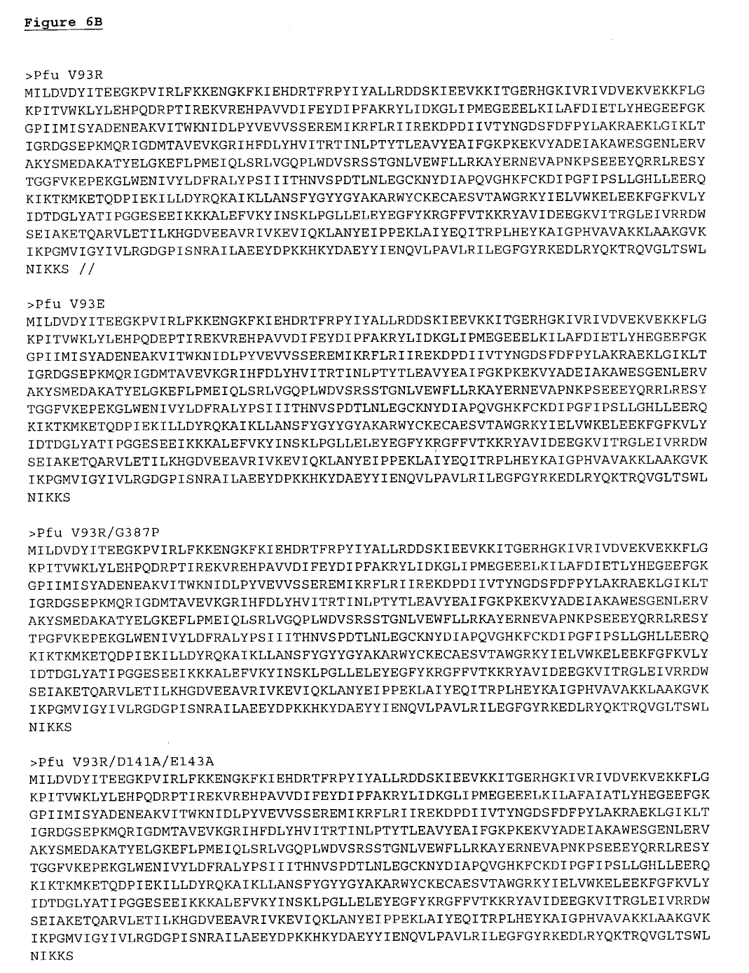

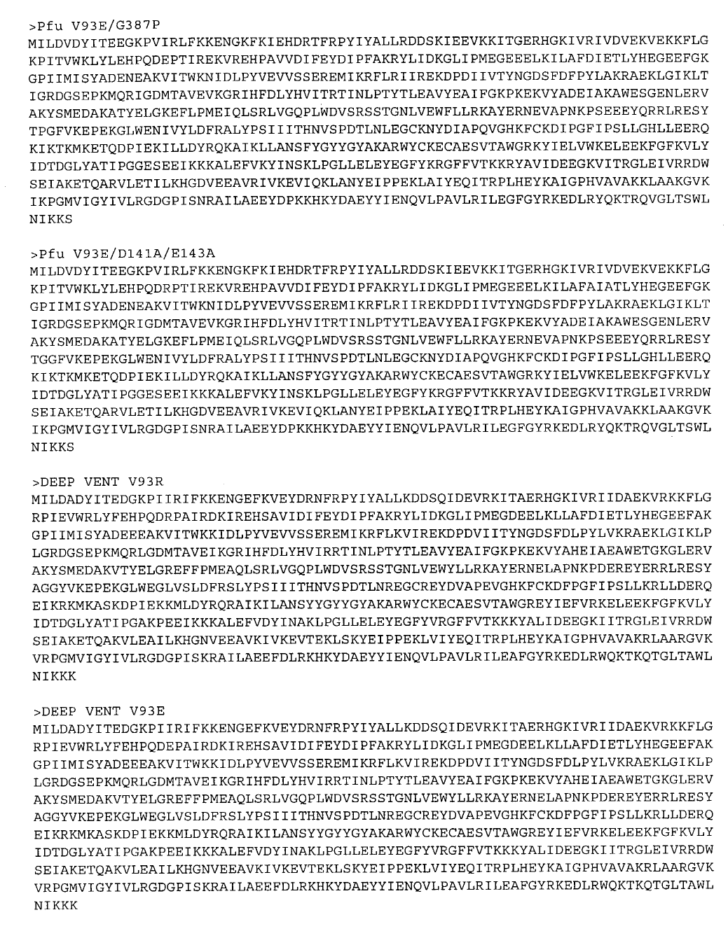

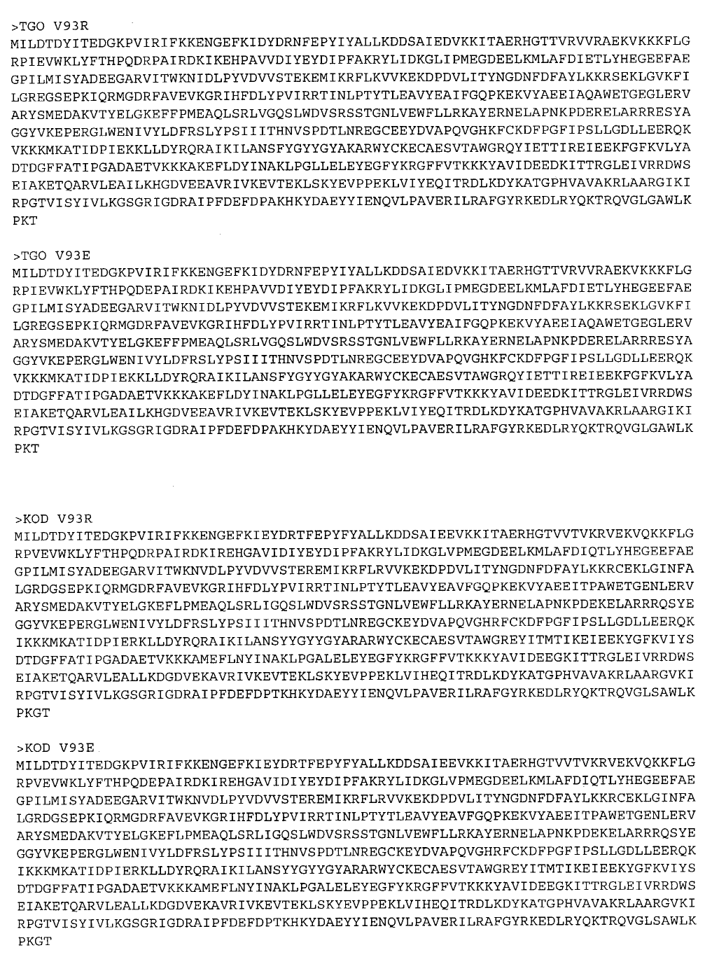

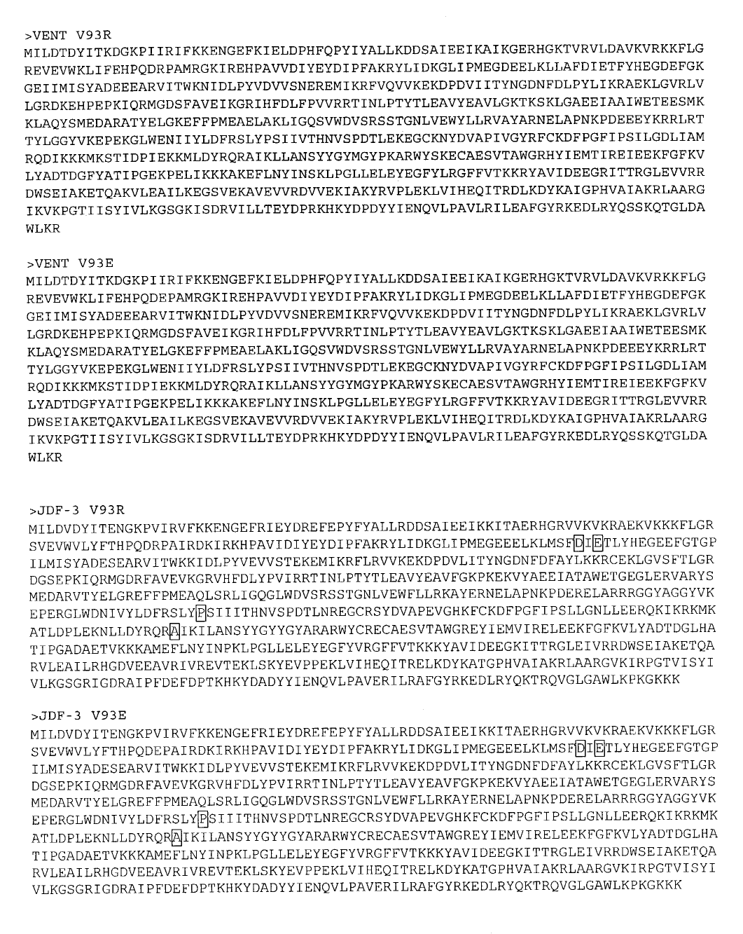

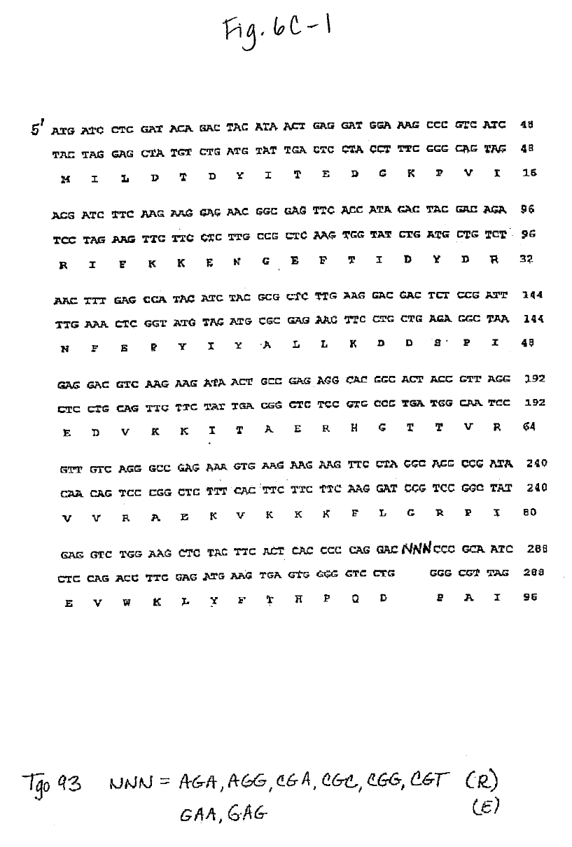

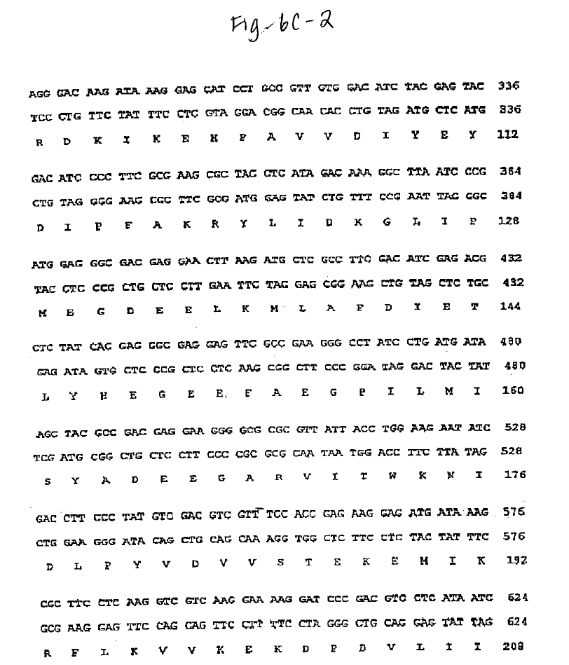

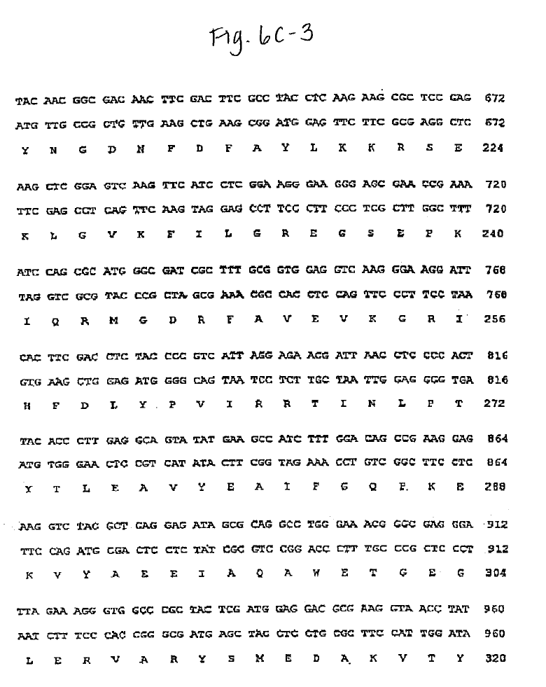

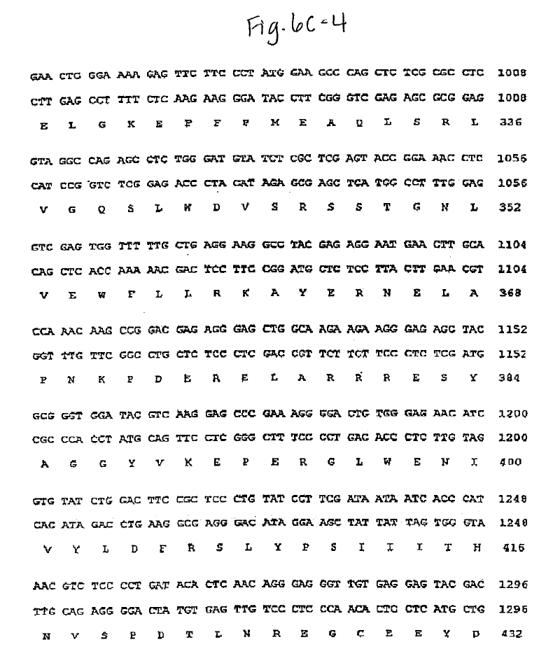

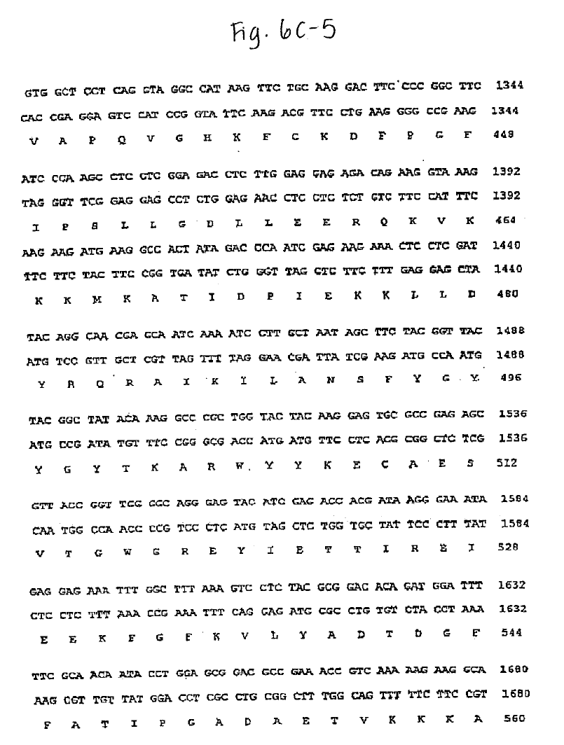

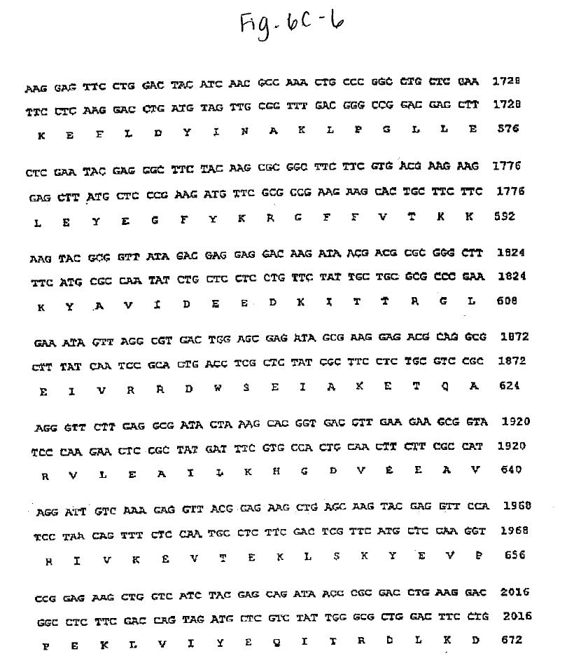

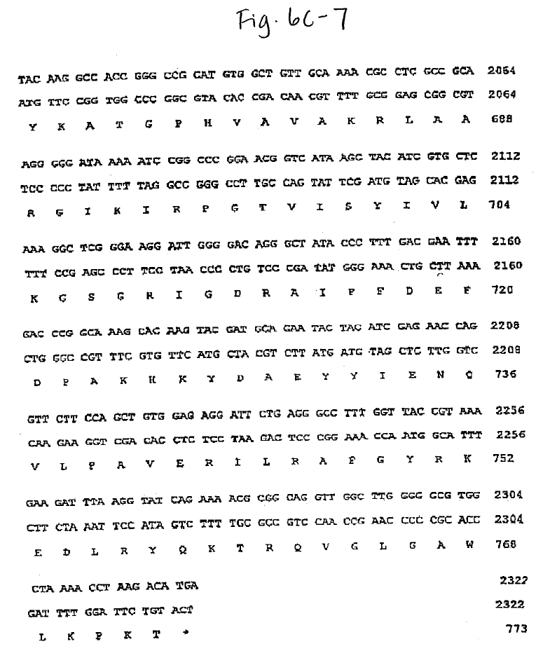

The invention further provides for mutant archaeal DNA polymerases with reduced base analog detection activity that contains a Valine to Arginine, Valine to Glutamic acid, Valine to Lysine, Valine to Aspartic Acid or Valine to Asparagine substitution at amino acid position 93 (see FIG. 6).

According to the invention, V93 mutant Pfu DNA polymerases with reduced uracil detection activity may contain one or more additional mutations that reduce or abolish one or more additional activities of V93 Pfu DNA polymerases, e.g., DNA polymerization activity or 3′-5′ exonuclease activity. In one embodiment, the V93 mutant Pfu DNA polymerase according to the invention contains one or more mutations that renders the DNA polymerase 3′-5′ exonuclease deficient. In another embodiment, the V93 mutant Pfu DNA polymerase according to the invention contains one or more mutations that the DNA polymerization activity of the V93 Pfu DNA polymerase.

In another embodiment, a mutant archael DNA polymerase is a chimera that further comprises a polypeptide that increases processivity and/or increases salt resistance. A polypeptide useful according to the invention and methods of preparing chimeras are described in WO 01/92501 A1 and Pavlov et al., 2002, Proc. Natl. Acad. Sci USA, 99:13510-13515. Both references are herein incorporated in their entirety.

The invention provides for V93R mutant Pfu DNA polymerases with reduced uracil detection activity containing one or mutations that reduce DNA polymerization as disclosed in the pending U.S. patent application Ser. No. 10/035,091 (Hogrefe, et al.; filed: Dec. 21, 2001); the pending U.S. patent application Ser. No. 10/079,241 (Hogrefe, et al.; filed Feb. 20, 2002); the pending U.S. patent application Ser. No. 10/208,508 (Hogrefe et al.; filed Jul. 30, 2002); and the pending U.S. patent application Ser. No. 10/227,110 (Hogrefe et al.; filed Aug. 23, 2002), the contents of which are hereby incorporated in their entirety.

In a preferred embodiment, the invention provides for a V93R/G387P, V93E/G387P, V93D/G387P, V93K/G387P and V93N/G387P double mutant Pfu DNA polymerase with reduced DNA polymerization activity and reduced uracil detection activity.

The invention further provides for V93R, V93E, V93D, V93K and V93N mutant Pfu DNA polymerases with reduced uracil detection activity containing one or mutations that reduce or eliminate 3′-5′ exonuclease activity as disclosed in the pending U.S. patent application Ser. No. 09/698,341 (Sorge et al; filed Oct. 27, 2000).

In a preferred embodiment, the invention provides for a V93R/D141A/E143A triple mutant Pfu DNA polymerase with reduced 3′-5′ exonuclease activity and reduced uracil detection activity.

The invention further provides for combination of one or more mutations that may increase or eliminate base analog detection activity of an archaeal DNA polymerase.

DNA polymerases containing additional mutations are generated by site directed mutagenesis using the Pfu DNA polymerase or Pfu V93R cDNA as a template DNA molecule, according to methods that are well known in the art and are described herein.

Methods used to generate Pfu DNA polymerases with reduced DNA polymerization activity are disclosed in the pending U.S. patent application Ser. No. 10/035,091 (Hogrefe, et al.; filed: Dec. 21, 2001); the pending U.S. patent application Ser. No. 10/079,241 (Hogrefe, et al.; filed Feb. 20, 2002); the pending U.S. patent application Ser. No. 10/208,508 (Hogrefe et al.; filed Jul. 30, 2002); and the pending U.S. patent application Ser. No. 10/227,110 (Hogrefe et al.; filed Aug. 23, 2002), the contents of which are hereby incorporated in their entirety.

Methods used to generate 3′-5′ exonuclease deficient JDF-3 DNA polymerases including the D141A and E143A mutations are disclosed in the pending U.S. patent application Ser. No. 09/698,341 (Sorge et al; filed Oct. 27, 2000). A person skilled in the art in possession of the V93 Pfu DNA polymerase cDNA and the teachings of the pending U.S. patent application Ser. No. 09/698,341 (Sorge et al; filed Oct. 27, 2000) would have no difficulty introducing both the corresponding D141A and E143A mutations or other 3′-5′ exonuclease mutations into the V93 Pfu DNA polymerase cDNA, as disclosed in the pending U.S. patent application Ser. No. 09/698,341, using established site directed mutagenesis methodology.

Methods of preparing chimeras according to the invention are described in WO 01/92501 A1 and Pavlov et al., 2002, Proc. Natl. Acad. Sci USA, 99:13510-13515. Both references are herein incorporated in their entirety.

In one embodiment, the Pfu mutants are expressed and purified as described in U.S. Pat. No. 5,489,523, hereby incorporated by reference in its entirety.

III. Methods of Evaluating Mutants for Reduced Base Analog Detection Activity.

Random or site-directed mutants generated as known in the art or as described herein and expressed in bacteria may be screened for reduced uracil detection activity by several different assays. Embodiments for the expression of mutant and wild type enzymes is described herein. In one method, exo+DNA polymerase proteins expressed in lytic lambda phage plaques generated by infection of host bacteria with expression vectors based on, for example, Lambda ZapII®, are transferred to a membrane support. The immobilized proteins are then assayed for polymerase activity on the membrane by immersing the membranes in a buffer containing a DNA template and the unconventional nucleotides to be monitored for incorporation.

Mutant polymerase libraries may be screened using a variation of the technique used by Sagner et al (Sagner, G., Ruger, R., and Kessler, C. (1991) Gene 97:119-123). For this approach, lambda phage clones are plated at a density of 10-20 plaques per square centimeter and replica plated. Proteins present in the plaques are transferred to filters and moistened with polymerase screening buffer (50 mM Tris (pH 8.0), 7 mM MgCl2, 3 mM β-ME). The filters are kept between layers of plastic wrap and glass while the host cell proteins are heat-inactivated by incubation at 65° C. for 30 minutes. The heat-treated filters are then transferred to fresh plastic wrap and approximately 35 μl of polymerase assay cocktail are added for every square centimeter of filter. The assay cocktail consists of 1× cloned Pfu (cPfu) magnesium free buffer (1× buffer is 20 mM Tris-HCl (pH 8.8), 10 mM KCl, 10 mM (NH4)₂SO₄, 100 μg/ml bovine serum albumin (BSA), and 0.1% Triton X-100; Pfu Magnesium-free buffer may be obtained from Stratagene (Catalog No. 200534)), 125 ng/ml activated calf thymus or salmon sperm DNA, 200 μM dATP, 2001M dGTP, 200 μM dCTP and 5 μCi/ml α-³³P dCTP and 200 μM dUTP or 200 μM dTTP. The filters, in duplicate, are placed between plastic wrap and a glass plate and then incubated at 65° C. for one hour, and then at 70° C. for one hour and fifteen minutes. Filters are then washed three times in 2×SSC for five minutes per wash before rinsing twice in 100% ethanol and vacuum drying. Filters are then exposed to X-ray film (approximately 16 hours), and plaques that incorporate label in the presence of 200 μM dUTP or 200 μM dTTP are identified by aligning the filters with the original plate bearing the phage clones. Plaques identified in this way are re-plated at more dilute concentrations and assayed under similar conditions to allow the isolation of purified plaques.

In assays such as the one described above, the signal generated by the label is a direct measurement of the polymerization activity of the polymerase in the presence of 200 μM dUTP as compared to the polymerase activity of the same mutant polymerase in the presence of 200 μM dTTP. A plaque comprising a mutant DNA polymerase with reduced uracil detection activity as compared to that of the wild-type enzyme can then be identified and further tested in primer extension assays in which template dependent DNA synthesis is measured in the presence of 200 μM dUTP. For example, 1 μl of appropriately diluted bacterial extract (i.e., heat-treated and clarified extract of bacterial cells expressing a cloned polymerase or mutated cloned polymerase) is added to 10 μl of each nucleotide cocktail (200 μM dATP, 200 μM dGTP, 200 μM dCTP and 5 μCi/ml α-³³P dCTP, 3H-dCTP and 200 μM dUTP or 200 μM dTTP, activated calf thymus DNA, 1× appropriate buffer (see above)), followed by incubation at the optimal temperature for 30 minutes (e.g., 73° C. for Pfu DNA polymerase), for example, as described in Hogrefe et al., 2001, Methods in Enzymology, 343:91-116. Extension reactions are then quenched on ice, and 5 μl aliquots are spotted immediately onto DE81 ion-exchange filters (2.3 cm; Whatman #3658323). Unincorporated label is removed by 6 washes with 2×SCC (0.3M NaCl, 30 mM sodium citrate, pH 7.0), followed by a brief wash with 100% ethanol. Incorporated radioactivity is then measured by scintillation counting. Reactions that lack enzyme are also set up along with sample incubations to determine “total cpms” (omit filter wash steps) and “minimum cpms”(wash filters as above). Cpms bound is proportional to the amount of polymerase activity present per volume of bacterial extract. Mutants that can incorporate significant radioactivity in the presence of dUTP are selected for further analysis.

Mutant DNA polymerases with reduced uracil recognition can also be identified as those that can synthesize PCR products in the presence of 100% dUTP (See Example 3).

The “uracil detection” activity can also be determined using the long range primer extension assay on single uracil templates as described by Greagg et al., (1999) Proc. Natl. Acad. Sci. 96, 9045-9050. Briefly, the assay requires a 119-mer template that is generated by PCR amplification of a segment of pUC19 spanning the polylinker cloning site. PCR primer sequences are:

| | | |
|---|---|---|
| A, | GACGTTGTAAAACGACGGCCAGU; | (SEQ ID NO:3) |
| B, | ACGTTGTAAAACGACGGCCAGT: AND | (SEQ ID NO:4) |
| C, | CAATTTCACACAGGAAACAGCTATGACCATG. | (SEQ ID NO:5) |

The 119-mer oligonucleotide incorporating either a U or T nucleotide 23 bases from the terminus of one strand, was synthesized by using Taq polymerase under standard PCR conditions, using primer C and either primer A or primer B. PCR products are then purified on agarose gels and extracted by using Qiagen columns.

For long range primer extension, primer C is annealed to one strand of the 119-bp PCR product by heating to 65° C. in reaction buffer and cooling to room temperature. The dNTPs, [α-[³²P] dATP, and 5 units of DNA polymerase (Pfu, Taq and mutant Pfu DNA polymerase to be tested) are added in polymerase reaction buffer (as specified by the suppliers of each polymerase) to a final volume of 20 μl, and the reaction is allowed to proceed for 60 min at 55° C. Reaction products are subjected to electrophoresis in a denaturing acrylamide gel and scanned and recorded on a Fuji FLA-2000 phosphorimager. The ability of the DNA polymerases from the thermophilic archaea Pyrococcus furiosus (Pfu) and the test mutant Pfu DNA polymerase to extend a primer across a template containing a single deoxyuridine can then be determined and directly compared.

IV. Expression of Wild-Type or Mutant Enzymes According to the Invention

Methods known in the art may be applied to express and isolate the mutated forms of DNA polymerase (i.e., the second enzyme) according to the invention. The methods described here can be also applied for the expression of wild-type enzymes useful (e.g., the first enzyme) in the invention. Many bacterial expression vectors contain sequence elements or combinations of sequence elements allowing high level inducible expression of the protein encoded by a foreign sequence. For example, as mentioned above, bacteria expressing an integrated inducible form of the T7 RNA polymerase gene may be transformed with an expression vector bearing a mutated DNA polymerase gene linked to the T7 promoter. Induction of the T7 RNA polymerase by addition of an appropriate inducer, for example, isopropyl-β-D-thiogalactopyranoside (IPTG) for a lac-inducible promoter, induces the high level expression of the mutated gene from the T7 promoter.

Appropriate host strains of bacteria may be selected from those available in the art by one of skill in the art. As a non-limiting example, E. coli strain BL-21 is commonly used for expression of exogenous proteins since it is protease deficient relative to other strains of E. coli. BL-21 strains bearing an inducible T7 RNA polymerase gene include WJ56 and ER2566 (Gardner & Jack, 1999, supra). For situations in which codon usage for the particular polymerase gene differs from that normally seen in E. coli genes, there are strains of BL-21 that are modified to carry tRNA genes encoding tRNAs with rarer anticodons (for example, argU, ileY, leuW, and proL tRNA genes), allowing high efficiency expression of cloned protein genes, for example, cloned archaeal enzyme genes (several BL21-CODON PLUS™ cell strains carrying rare-codon tRNAs are available from Stratagene, for example).

There are many methods known to those of skill in the art that are suitable for the purification of a modified DNA polymerase of the invention. For example, the method of Lawyer et al. (1993, PCR Meth. & App. 2: 275) is well suited for the isolation of DNA polymerases expressed in E. coli, as it was designed originally for the isolation of Taq polymerase. Alternatively, the method of Kong et al. (1993, J. Biol. Chem. 268: 1965, incorporated herein by reference) may be used, which employs a heat denaturation step to destroy host proteins, and two column purification steps (over DEAE-Sepharose and heparin-Sepharose columns) to isolate highly active and approximately 80% pure DNA polymerase. Further, DNA polymerase mutants may be isolated by an ammonium sulfate fractionation, followed by Q Sepharose and DNA cellulose columns, or by adsorption of contaminants on a HiTrap Q column, followed by gradient elution from a HiTrap heparin column.

The invention further provides for mutant V93R, V93E, V93D, V93K or V93N Pfu DNA polymerases that contain one or more additional mutations with improved reverse transcriptase activity.

The invention further provides for compositions in which V93 archaeal or Pfu mutant DNA polymerases with reduced base analog detection activity contain additional mutations that reduced DNA polymerization activity for example, G387P (polymerase minus) or 3′-5′ exonuclease activity, for example, D141A/E143A (3′-5′ exonuclease minus) The invention further provides for compositions comprising mutant archeal polymerases that are chimeras, as described herein.

The invention provides for compositions wherein n the archael or Pfu mutant DNA polymerases are mixed as described in Table 2.

| | | | | | Pfu V93R- |
|---|---|---|---|---|---|
| | | | Pfu-chimera + | Pfu V93R- | chimera + |
| | None | PEF | PEF | chimera | PEF |
| Pfu V93R | Yes | Yes | Yes | Yes | Yes |
| Pfu | | | | Yes | Yes |
| Pfu V93R + | | Yes | Yes | Yes | Yes |
| Pfu G387P | | | | | |
| Pfu V93R + | Yes | Yes | Yes | Yes | Yes |
| Pfu V93R/G387P | | | | | |
| Taq + Pfu | Yes | Yes | Yes | Yes | Yes |
| V93R | | | | | |
| Taq + Pfu | | | | Yes | Yes |
| Taq + Pfu | Yes | Yes | Yes | Yes | Yes |
| V93R + | | | | | |
| Pfu G387P | | | | | |
| Taq + Pfu | Yes | Yes | Yes | Yes | Yes |
| V93R + Pfu | | | | | |
| V93R/G387P | | | | | |
| Taq + Pfu + | | | | Yes | Yes |
| Pfu G387P | | | | | |
| Taq + Pfu + | | | | | |
| Pfu V93R/G387P | | | | | |
| Taq + Pfu | Yes | Yes | | | |
| V93R/G387P | | | | | |
| Pfu | Yes | Yes | | | |
| V93R/D141 | | | | | |
| A/E143A | | | | | |

The invention further provides for compositions in which any of the archaeal or Pfu mutant DNA polymerases with reduced base analog detection activity are mixed with either

a) Pfu G387P (polymerase minus)

b) Taq polymerase

c) PEF

d) a Pfu polymerase chimera (as described in WO 01/92501 A1 or Pavlov et al. supra)

e) a mutant archael Pfu polymerase chimera as described herein

f) Pfu g387P/V93R double mutant.

The invention also provides for mixtures of V93 mutant archaeal or Pfu DNA polymerases, preferably V93R, with additional compositions that include, but are not limited to:

A.) blended with PCR enhancing factor (PEF)

B.) blended with Taq (at any ratio, but preferably a higher ratio of Pfu mutant to Taq) with or without PEF

C.) blended with Pfu G387P, Pfu G387P/V93R, G387P/V93E, Pfu G387P/V93D, Pfu G387P/V93K or G387P/V93N mutants (for higher fidelity PCR)

D.) blended with Thermus DNA ligase and FEN-1 (for multisite site-directed mutagenesis)

E.) blended with a Pfu polymerase chimera (as described in WO 01/92501 A1 or Pavlov et al. supra) or a mutant archael Pfu polymerase chimera as described herein

F.) blended with additives like antibodies for increased specificity (for hot start PCR, described in Borns et al. (2001) Strategies 14, pages 5-8 and also in manual accompanying commercially available kit, Stratagene Catalogue # 600320), DMSO for GC-rich PCR or single stranded DNA binding protein for higher specificity (commercially available, Stratagene Catalog # 600201)

The invention also contemplates a mixture comprising the combination of a mutant archael DNA polymerase of the invention, dUTP and uracil N-glycosylase.

The invention further provides for the archaeal DNA polymerases of the invention with reduced base analog detection activity be combined with the Easy A composition that contains a blend of Taq (5 U/ul), recombinant PEF (4 U/ul), and Pfu G387P/V93R, E, N, D, K or N mutant (40 ng/ul) as disclosed in the pending U.S. patent application Ser. No. 10/035,091 (Hogrefe, et al.; filed: Dec. 21, 2001); the pending U.S. patent application Ser. No. 10/079,241 (Hogrefe, et al.; filed Feb. 20, 2002); the pending U.S. patent application Ser. No. 10/208,508 (Hogrefe et al.; filed Jul. 30, 2002); and the pending U.S. patent application Ser. No. 10/227,110 (Hogrefe et al.; filed Aug. 23, 2002), the contents of which are hereby incorporated in their entirety. With cloned archaeal DNA polymerase with reduced base analog detection activity at 2.5 U/ul i.e. ˜20-50 ng per ul, the ratio of Taq:Pfu is preferably 1:1 or more preferably 2:1 or more.

V. Applications of the Subject Invention

In one aspect, the invention provides a method for DNA synthesis using the compositions of the subject invention. Typically, synthesis of a polynucleotide requires a synthesis primer, a synthesis template, polynucleotide precursors for incorporation into the newly synthesized polynucleotide, (e.g. dATP, dCTP, dGTP, dTTP), and the like. Detailed methods for carrying out polynucleotide synthesis are well known to the person of ordinary skill in the art and can be found, for example, in Molecular Cloning second edition, Sambrook et al., Cold Spring Harbor Laboratory Press, Cold Spring Harbor, N.Y. (1989).

A. Application in Amplification Reactions

“Polymerase chain reaction” or “PCR” refers to an in vitro method for amplifying a specific polynucleotide template sequence. The technique of PCR is described in numerous publications, including, PCR: A Practical Approach, M. J. McPherson, et al., IRL Press (1991 ), PCR Protocols: A Guide to Methods and Applications, by Innis, et al., Academic Press (1990), and PCR Technology: Principals and Applications for DNA Amplification, H. A. Erlich, Stockton Press (1989). PCR is also described in many U.S. patents, including U.S. Pat. Nos. 4,683,195; 4,683,202; 4,800,159; 4,965,188; 4,889,818; 5,075,216; 5,079,352; 5,104,792; 5,023,171; 5,091,310; and 5,066,584, each of which is herein incorporated by reference.

For ease of understanding the advantages provided by the present invention, a summary of PCR is provided. The PCR reaction involves a repetitive series of temperature cycles and is typically performed in a volume of 50-100 μl. The reaction mix comprises dNTPs (each of the four deoxynucleotides dATP, dCTP, dGTP, and dTTP), primers, buffers, DNA polymerase, and polynucleotide template. PCR requires two primers that hybridize with the double-stranded target polynucleotide sequence to be amplified. In PCR, this double-stranded target sequence is denatured and one primer is annealed to each strand of the denatured target. The primers anneal to the target polynucleotide at sites removed from one another and in orientations such that the extension product of one primer, when separated from its complement, can hybridize to the other primer. Once a given primer hybridizes to the target sequence, the primer is extended by the action of a DNA polymerase. The extension product is then denatured from the target sequence, and the process is repeated.

In successive cycles of this process, the extension products produced in earlier cycles serve as templates for DNA synthesis. Beginning in the second cycle, the product of amplification begins to accumulate at a logarithmic rate. The amplification product is a discrete double-stranded DNA molecule comprising: a first strand which contains the sequence of the first primer, eventually followed by the sequence complementary to the second primer, and a second strand which is complementary to the first strand.

Due to the enormous amplification possible with the PCR process, small levels of DNA carryover from samples with high DNA levels, positive control templates or from previous amplifications can result in PCR product, even in the absence of purposefully added template DNA. If possible, all reaction mixes are set up in an area separate from PCR product analysis and sample preparation. The use of dedicated or disposable vessels, solutions, and pipettes (preferably positive displacement pipettes) for RNA/DNA preparation, reaction mixing, and sample analysis will minimize cross contamination. See also Higuchi and Kwok, 1989, Nature, 339:237-238 and Kwok, and Orrego, in: Innis et al. eds., 1990, PCR Protocols: A Guide to Methods and Applications, Academic Press, Inc., San Diego, Calif., which are incorporated herein by reference.

The enzymes provided herein are also useful for dUTP/UNG cleanup methods that require PCR enzymes that incorporate dUTP (Longo et al., Supra).

1. Thermostable Enzymes

For PCR amplifications, the enzymes used in the invention are preferably thermostable. As used herein, “thermostable” refers to an enzyme which is stable to heat, is heat resistant, and functions at high temperatures, e.g., 50 to 90° C. The thermostable enzyme according to the present invention must satisfy a single criterion to be effective for the amplification reaction, i.e., the enzyme must not become irreversibly denatured (inactivated) when subjected to the elevated temperatures for the time necessary to effect denaturation of double-stranded polynucleotides. By “irreversible denaturation” as used in this connection, is meant a process bringing a permanent and complete loss of enzymatic activity. The heating conditions necessary for denaturation will depend, e.g., on the buffer salt concentration and the length and nucleotide composition of the polynucleotides being denatured, but typically range from 85° C., for shorter polynucleotides, to 105° C. for a time depending mainly on the temperature and the polynucleotide length, typically from 0.25 minutes for shorter polynucleotides, to 4.0 minutes for longer pieces of DNA. Higher temperatures may be tolerated as the buffer salt concentration and/or GC composition of the polynucleotide is increased. Preferably, the enzyme will not become irreversibly denatured at 90 to 100° C. An enzyme that does not become irreversibly denatured, according to the invention, retains at least 10%, or at least 25%, or at least 50% or more function or activity during the amplification reaction.

2. PCR Reaction Mixture

In addition to the subject enzyme mixture, one of average skill in the art may also employ other PCR parameters to increase the fidelity of synthesis/amplification reaction. It has been reported PCR fidelity may be affected by factors such as changes in dNTP concentration, units of enzyme used per reaction, pH, and the ratio of Mg²⁺ to dNTPs present in the reaction (Mattila et al., 1991, supra).

Mg²⁺ concentration affects the annealing of the oligonucleotide primers to the template DNA by stabilizing the primer-template interaction, it also stabilizes the replication complex of polymerase with template-primer. It can therefore also increases non-specific annealing and produced undesirable PCR products (gives multiple bands in gel). When non-specific amplification occurs, Mg²⁺ may need to be lowered or EDTA can be added to chelate Mg²⁺ to increase the accuracy and specificity of the amplification.

Other divalent cations such as Mn²⁺, or Co²⁺ can also affect DNA polymerization. Suitable cations for each DNA polymerase are known in the art (e.g., in DNA Replication 2^{nd} edition, supra). Divalent cation is supplied in the form of a salt such MgCl₂, Mg(OAc)₂, MgSO₄, MnCl₂, Mn(OAc)₂, or MnSO₄. Usable cation concentrations in a Tris-HCl buffer are for MnCl₂ from 0.5 to 7 mM, preferably, between 0.5 and 2 mM, and for MgCl₂ from 0.5 to 10 mM. Usable cation concentrations in a Bicine/KOAc buffer are from 1 to 20 mM for Mn(OAc)₂, preferably between 2 and 5 mM.

Monovalent cation required by DNA polymerase may be supplied by the potassium, sodium, ammonium, or lithium salts of either chloride or acetate. For KCl, the concentration is between 1 and 200 mM, preferably the concentration is between 40 and 100 mM, although the optimum concentration may vary depending on the polymerase used in the reaction.

Deoxyribonucleotide triphosphates (dNTPs) are added as solutions of the salts of dATP, dCTP, dGTP, dUTP, and dTTP, such as disodium or lithium salts. In the present methods, a final concentration in the range of 1 μM to 2 mM each is suitable, and 100-600 μM is preferable, although the optimal concentration of the nucleotides may vary in the PCR reaction depending on the total dNTP and divalent metal ion concentration, and on the buffer, salts, particular primers, and template. For longer products, i.e., greater than 1500 bp, 500 μM each dNTP may be preferred when using a Tris-HCl buffer.

dNTPs chelate divalent cations, therefore amount of divalent cations used may need to be changed according to the dNTP concentration in the reaction. Excessive amount of dNTPs (e.g., larger than 1.5 mM) can increase the error rate and possibly inhibit DNA polymerases. Lowering the dNTP (e.g., to 10-50 μM) may therefore reduce error rate. PCR reaction for amplifying larger size template may need more dNTPs.

One suitable buffering agent is Tris-HCl, preferably pH 8.3, although the pH may be in the range 8.0-8.8. The Tris-HCl concentration is from 5-250 mM, although 10-100 mM is most preferred. A preferred buffering agent is Bicine-KOH, preferably pH 8.3, although pH may be in the range 7.8-8.7. Bicine acts both as a pH buffer and as a metal buffer. Tricine may also be used.

PCR is a very powerful tool for DNA amplification and therefore very little template DNA is needed. However, in some embodiments, to reduce the likelihood of error, a higher DNA concentration may be used, though too many templates may increase the amount of contaminants and reduce efficiency.

Usually, up to 3 μM of primers may be used, but high primer to template ratio can results in non-specific amplification and primer-dimer formation. Therefore it is usually necessary to check primer sequences to avoid primer-dimer formation.

The invention provides for Pfu V93R, V93E, V93K, V93D, or V93N DNA polymerases with reduced uracil detection activity that enhance PCR of GC rich DNA templates by minimizing the effect of cytosine deamination in the template and by allowing the use of higher denaturation times and denaturation temperatures.

3. Cycling Parameters

Denaturation time may be increased if template GC content is high. Higher annealing temperature may be needed for primers with high GC content or longer primers. Gradient PCR is a useful way of determining the annealing temperature. Extension time should be extended for larger PCR product amplifications. However, extension time may need to be reduced whenever possible to limit damage to enzyme.

The number of cycle can be increased if the number of template DNA is very low, and decreased if high amount of template DNA is used.

4. PCR Enhancing Factors and Additives

PCR enhancing factors may also be used to improve efficiency of the amplification. As used herein, a “PCR enhancing factor” or a “Polymerase Enhancing Factor” (PEF) refers to a complex or protein possessing polynucleotide polymerase enhancing activity (Hogrefe et al., 1997, Strategies 10:93-96; and U.S. Pat. No. 6,183,997, both of which are hereby incorporated by references). For Pfu DNA polymerase, PEF comprises either P45 in native form (as a complex of P50 and P45) or as a recombinant protein. In the native complex of Pfu P50 and P45, only P45 exhibits PCR enhancing activity. The P50 protein is similar in structure to a bacterial flavoprotein. The P45 protein is similar in structure to dCTP deaminase and dUTPase, but it functions only as a dUTPase converting dUTP to dUMP and pyrophosphate. PEF, according to the present invention, can also be selected from the group consisting of: an isolated or purified naturally occurring polymerase enhancing protein obtained from an archeabacteria source (e.g., Pyrococcus furiosus); a wholly or partially synthetic protein having the same amino acid sequence as Pfu P45, or analogs thereof possessing polymerase enhancing activity; polymerase-enhancing mixtures of one or more of said naturally occurring or wholly or partially synthetic proteins; polymerase-enhancing protein complexes of one or more of said naturally occurring or wholly or partially synthetic proteins; or polymerase-enhancing partially purified cell extracts containing one or more of said naturally occurring proteins (U.S. Pat. No. 6,183,997, supra). The PCR enhancing activity of PEF is defined by means well known in the art. The unit definition for PEF is based on the dUTPase activity of PEF (P45), which is determined by monitoring the production of pyrophosphate (PPi) from dUTP. For example, PEF is incubated with dUTP (10 mM dUTP in 1× cloned Pfu PCR buffer) during which time PEF hydrolyzes dUTP to dUMP and PPi. The amount of PPi formed is quantitated using a coupled enzymatic assay system that is commercially available from Sigma (#P7275). One unit of activity is functionally defined as 4.0 nmole of PPi formed per hour (at 85° C.).

Other PCR additives may also affect the accuracy and specificity of PCR reaction. EDTA less than 0.5 mM may be present in the amplification reaction mix. Detergents such as Tween-20™ and Nonidet™ P-40 are present in the enzyme dilution buffers. A final concentration of non-ionic detergent approximately 0.1% or less is appropriate, however, 0.01-0.05% is preferred and will not interfere with polymerase activity. Similarly, glycerol is often present in enzyme preparations and is generally diluted to a concentration of 1-20% in the reaction mix. Glycerol (5-10%), formamide (1-5%) or DMSO (2-10%) can be added in PCR for template DNA with high GC content or long length (e.g., >1 kb). These additives change the Tm (melting temperature) of primer-template hybridization reaction and the thermostability of polymerase enzyme. BSA (up to 0.8 μg/μl) can improve efficiency of PCR reaction. Betaine (0.5-2M) is also useful for PCR over high GC content and long fragments of DNA. Tetramethylammonium chloride (TMAC, >50 mM), Tetraethylammonium chloride (TEAC), and Trimethlamine N-oxide (TMANO) may also be used. Test PCR reactions may be performed to determine optimum concentration of each additive mentioned above.

The invention provides for additive including, but not limited to antibodies (for hot start PCR) and ssb (higher specificity). The invention also contemplates mutant archael DNA polymerases in combination with accessory factors, for example as described in U.S. Pat. No. 6,333,158, and WO 01/09347 A2, hereby incorporated by reference in its entirety.

Various specific PCR amplification applications are available in the art (for reviews, see for example, Erlich, 1999, Rev Immunogenet., 1:127-34; Prediger 2001, Methods Mol. Biol. 160:49-63; Jurecic et al., 2000, Curr. Opin. Microbiol. 3:316-21; Triglia, 2000, Methods Mol. Biol. 130:79-83; MaClelland et al., 1994, PCR Methods Appl. 4:S66-81; Abramson and Myers, 1993, Current Opinion in Biotechnology 4:41-47; each of which is incorporated herein by references).

The subject invention can be used in PCR applications including, but are not limited to, i) hot-start PCR which reduces non-specific amplification; ii) touch-down PCR which starts at high annealing temperature, then decreases annealing temperature in steps to reduce non-specific PCR product; iii) nested PCR which synthesizes more reliable product using an outer set of primers and an inner set of primers; iv) inverse PCR for amplification of regions flanking a known sequence. In this method, DNA is digested, the desired fragment is circularized by ligation, then PCR using primer complementary to the known sequence extending outwards; v) AP-PCR (arbitrary primed)/RAPD (random amplified polymorphic DNA). These methods create genomic fingerprints from species with little-known target sequences by amplifying using arbitrary oligonucleotides; vi) RT-PCR which uses RNA-directed DNA polymerase (e.g., reverse transcriptase) to synthesize cDNAs which is then used for PCR. This method is extremely sensitive for detecting the expression of a specific sequence in a tissue or cells. It may also be use to quantify mRNA transcripts; vii) RACE (rapid amplification of cDNA ends). This is used where information about DNA/protein sequence is limited. The method amplifies 3′ or 5′ ends of cDNAs generating fragments of cDNA with only one specific primer each (plus one adaptor primer). Overlapping RACE products can then be combined to produce full length cDNA; viii) DD-PCR (differential display PCR) which is used to identify differentially expressed genes in different tissues. First step in DD-PCR involves RT-PCR, then amplification is performed using short, intentionally nonspecific primers; ix) Multiplex-PCR in which two or more unique targets of DNA sequences in the same specimen are amplified simultaneously. One DNA sequence can be use as control to verify the quality of PCR; x) Q/C-PCR (Quantitative comparative) which uses an internal control DNA sequence (but of different size) which compete with the target DNA (competitive PCR) for the same set of primers; xi) Recusive PCR which is used to synthesize genes. Oligonucleotides used in this method are complementary to stretches of a gene (>80 bases), alternately to the sense and to the antisense strands with ends overlapping (˜20 bases); xii) Asymmetric PCR; xiii) In Situ PCR; xiv) Site-directed PCR Mutagenesis.

It should be understood that this invention is not limited to any particular amplification system. As other systems are developed, those systems may benefit by practice of this invention.

B. Application in Direct Cloning of PCR Amplified Product

It is understood that the amplified product produced using the subject enzyme can be cloned by any method known in the art. In one embodiment, the invention provides a composition which allows direct cloning of PCR amplified product.

The most common method for cloning PCR products involves incorporation of flanking restriction sites onto the ends of primer molecules. The PCR cycling is carried out and the amplified DNA is then purified, restricted with an appropriate endonuclease(s) and ligated to a compatible vector preparation.

A method for directly cloning PCR products eliminates the need for preparing primers having restriction recognition sequences and it would eliminate the need for a restriction step to prepare the PCR product for cloning. Additionally, such method would preferably allow cloning PCR products directly without an intervening purification step.

U.S. Pat. Nos. 5,827,657 and 5,487,993 (hereby incorporated by their entirety) disclose methods for direct cloning of PCR products using a DNA polymerase which takes advantage of the single 3′-deoxy-adenosine monophosphate (dAMP) residues attached to the 3′ termini of PCR generated nucleic acids. Vectors are prepared with recognition sequences that afford single 3′-terminal deoxy-thymidine monophosphate (dTMP) residues upon reaction with a suitable restriction enzyme. Thus, PCR generated copies of genes can be directly cloned into the vectors without need for preparing primers having suitable restriction sites therein.

Taq DNA polymerase exhibits terminal transferase activity that adds a single dATP to the 3′ ends of PCR products in the absence of template. This activity is the basis for the TA cloning method in which PCR products amplified with Taq are directly ligated into vectors containing single 3′dT overhangs. Pfu DNA polymerase, on the other hand, lacks terminal transferase activity, and thus produces blunt-ended PCR products that are efficiently cloned into blunt-ended vectors.

In one embodiment, the invention provides for a PCR product, generated in the presence of a mutant DNA polymerase with reduced uracil detection activity, that is subsequently incubated with Taq DNA polymerase in the presence of dATP at 72° C. for 15-30 minutes. Addition of 3′-dAMP to the ends of the amplified DNA product then permits cloning into TA cloning vectors according to methods that are well known to a person skilled in the art.

C. Application in DNA Sequencing

The invention further provides for dideoxynucleotide DNA sequencing methods using thermostable DNA polymerases having a reduced base analog detection activity to catalyze the primer extension reactions. Methods for dideoxynucleotide DNA sequencing are well known in the art and are disclosed in U.S. Pat. Nos. 5,075,216, 4,795,699 and 5,885,813, the contents of which are hereby incorporated in their entirety.

D. Application in Mutagenesis

The mutant archaeal DNA polymerases of the invention, preferably V93R Pfu DNA polymerase, also provide enhanced efficacy for PCR-based or linear amplification-based mutagenesis. The invention therefore provides for the use of the mutant archaeal DNA polymerases with reduced base analog detection activity for site-directed mutagenesis and their incorporation into commercially available kits, for example, QuikChange Site-directed Mutagenesis, QuikChange Multi-Site-Directed Mutagenesis (Stratagene). Site-directed mutagenesis methods and reagents are disclosed in the pending U.S. patent application Ser. No. 10/198,449 (Hogrefe et al.; filed Jul. 18, 2002), the contents of which are hereby incorporated in its entirety.

VI. Kits

The invention herein also contemplates a kit format which comprises a package unit having one or more containers of the subject composition and in some embodiments including containers of various reagents used for polynucleotide synthesis, including synthesis in PCR. The kit may also contain one or more of the following items: polynucleotide precursors, primers, buffers, instructions, and controls. Kits may include containers of reagents mixed together in suitable proportions for performing the methods in accordance with the invention. Reagent containers preferably contain reagents in unit quantities that obviate measuring steps when performing the subject methods.

The invention contemplates a kit comprising a combination of a mutant archael DNA polymerase of the invention, dUTP and uracil N-glycosylase.

### VII. EXAMPLES

### Example 1

Construction of Pfu DNA Polymerase Mutants with Reduced Uracil Detection

Mutations were introduced into Pfu DNA polymerase that were likely to reduce uracil detection, while having minimal effects on polymerase or proofreading activity. The DNA template used for mutagenesis contained the Pfu pol gene, cloned into pBluescript (pF72 clone described in U.S. Pat. No. 5,489,523). Point mutations were introduced using the QuikChange or the QuikChange Multi Site-Directed Mutagenesis Kit (Stratagene). With the QuikChange kit, point mutations are introduced using a pair of mutagenic primers (V93E, H, K, R, and N). With the QuikChange Multi kit, specific point mutations are introduced by incorporating one phosphorylated mutagenic primer or by selecting random mutants from a library of Pfu V93 variants, created by incorporating a degenerate codon (V93G and L). Clones were sequenced to identify the incorporated mutations.

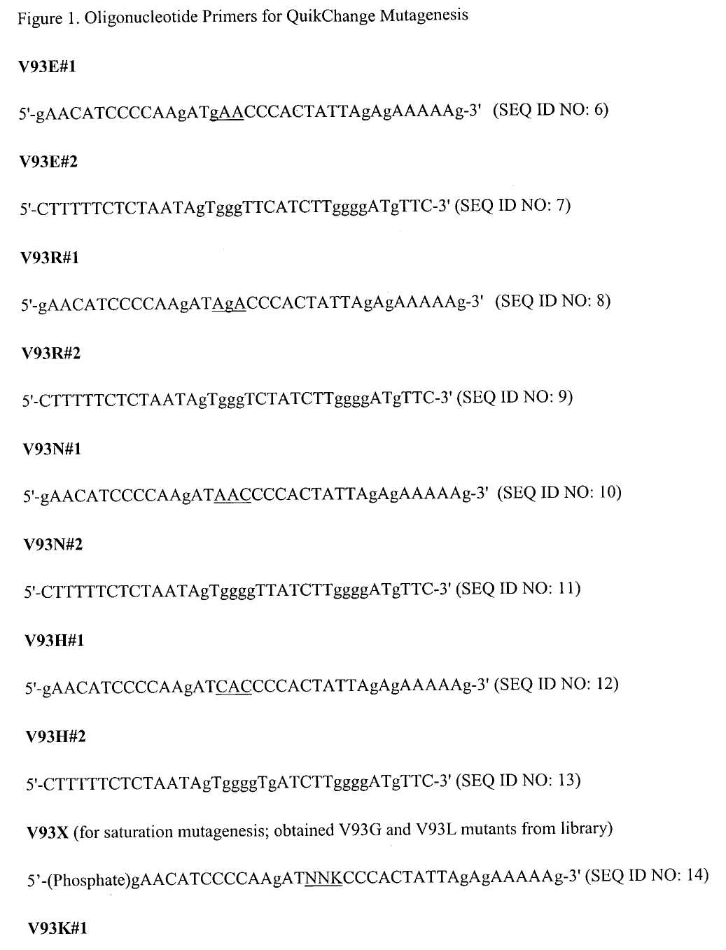

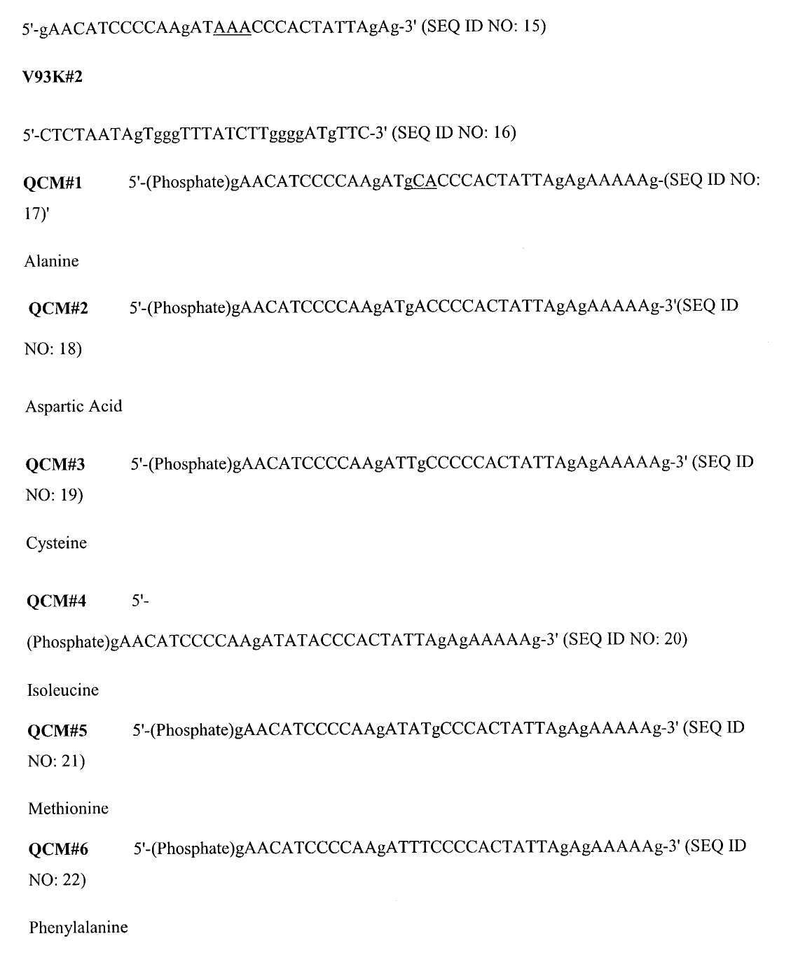

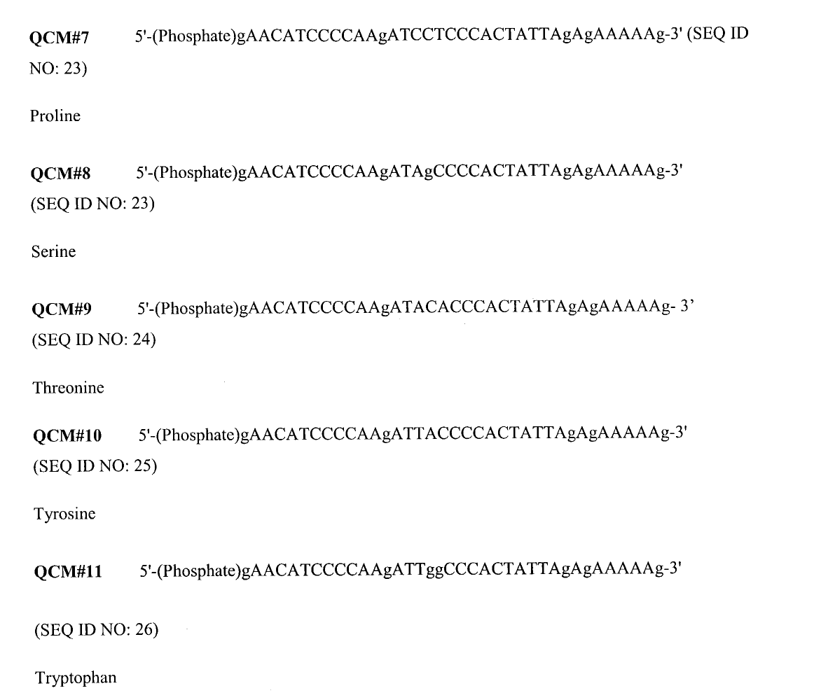

Results. Valine 93 in Pfu DNA polymerase was substituted with Glycine (G), asparagine (N), arginine [R], glutamic acid (E), histidine (H), and leucine (L) using the QuikChange primer sequences listed in FIG. 1.

### Example 2

Preparation of Bacterial Extracts Containing Mutant Pfu DNA Polymerases

Plasmid DNA was purified with the StrataPrep® Plasmid Miniprep Kit (Stratagene), and used to transform BL26-CodonPlus-RIL cells. Ampicillin resistant colonies were grown up in 1-5 liters of LB media containing Turbo Amp™ (100 μg/μl) and chloramphenicol (30 μg/μl) at 30° C. with moderate aeration. The cells were collected by centrifugation and stored at −80° C. until use.

Cell pellets (12-24 grams) were resuspended in 3 volumes of lysis buffer (buffer A: 50 mM Tris HCl (pH 8.2), 1 mM EDTA, and 10 mM βME). Lysozyme (1 mg/g cells) and PMSF (1 mM) were added and the cells were lysed for 1 hour at 4° C. The cell mixture was sonicated, and the debris removed by centrifugation at 15,000 rpm for 30 minutes (4° C.). Tween 20 and Igepal CA-630 were added to final concentrations of 0.1% and the supernatant was heated at 72° C. for 10 minutes. Heat denatured E. coli proteins were then removed by centrifugation at 15,000 rpm for 30 minutes (4° C.).

### Example 3

Assessment of dUTP Incorporation by PCR

Partially-purified Pfu mutant preparations (heat-treated bacterial extracts) were assayed for dUTP incorporation during PCR. In this example, a 2.3 kb fragment containing the Pfu pol gene was from plasmid DNA using PCR primers: (FPfuLIC) 5′-gACgACgACAAgATgATTTTAgATgTggAT-3′ and (RPfuLIC) 5′-ggAACAAgACCCgTCTAggATTTTTTAATg-3′. Amplification reactions consisted of 1× cloned Pfu PCR buffer, 7 ng plasmid DNA, 100 ng of each primer, 2.5 U of Pfu mutant (or wild type Pfu), and 200 μM each dGTP, dCTP, and dATP. To assess relative dUTP incorporation, various amounts of dUTP (0-400 μM) and/or TTP (0-200 μM) were added to the PCR reaction cocktail. The amplification reactions were cycled as described in example 6.

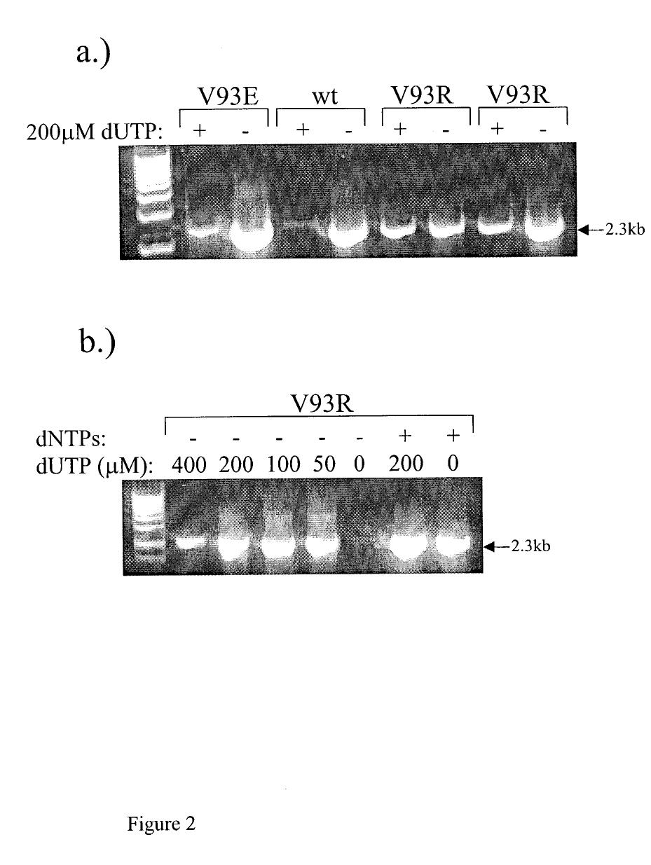

Results. Partially-purified preparations of the V93E and V93R mutants showed improved dUTP incorporation compared to wild type Pfu (FIG. 2a). Each mutant successfully amplified a 2.3 kb target in the presence of 200 μM dUTP (plus 200M each TTP, dATP, dCTP, dGTP). In contrast, extracts containing the Pfu V93N, V93G, V93H, and V93L mutants showed little-to-no amplification in the presence of 200 μM dUTP, similar to wild type Pfu (data not shown). Additional testing showed that the Pfu V93R mutant extract amplified the 2.3 kb target in the presence of 100% dUTP (0% TTP)(FIG. 2b).

### Example 4

Purification of Pfu DNA Polymerase Mutants

Bacterial expression of Pfu mutants. Pfu mutants can be purified as described in U.S. Pat. No. 5,489,523 (purification of the exo⁻ Pfu D141A/E143A DNA polymerase mutant) or as follows. Clarified, heat-treated bacterial extracts were chromatographed on a Q-Sepharose™ Fast Flow column (˜20 ml column), equilibrated in buffer B (buffer A plus 0.1% (v/v) Igepal CA-630, and 0.1% (v/v) Tween 20). Flow-through fractions were collected and then loaded directly onto a P11 Phosphocellulose column (˜20 ml), equilibrated in buffer C (same as buffer B, except pH 7.5). The column was washed and then eluted with a 0-0.7M KCl gradient/Buffer C. Fractions containing Pfu DNA polymerase mutants (95 kD by SDS-PAGE) were dialyzed overnight against buffer D (50 mM Tris HCl (pH 7.5), 5 mM βME, 5% (v/v) glycerol, 0.2% (v/v) Igepal CA-630, 0.2% (v/v) Tween 20, and 0.5M NaCl) and then applied to a Hydroxyapatite column (˜5 ml), equilibrated in buffer D. The column was washed and Pfu DNA polymerase mutants were eluted with buffer D2 containing 400 mM KPO₄, (pH 7.5), 5 mM βME, 5% (v/v) glycerol, 0.2% (v/v) Igepal CA-630, 0.2% (v/v) Tween 20, and 0.5 M NaCl. Purified proteins were spin concentrated using Centricon YM30 devices, and exchanged into Pfu final dialysis buffer (50 mM Tris-HCl (pH 8.2), 0.1 mM EDTA, 1 mM dithiothreitol (DTT), 50% (v/v) glycerol, 0.1% (v/v) Igepal CA-630, and 0.1% (v/v) Tween 20).

Protein samples were evaluated for size, purity, and approximate concentration by SDS-PAGE using Tris-Glycine 4-20% acrylamide gradient gels. Gels were stained with silver stain or Sypro Orange (Molecular Probes). Protein concentration was determined relative to a BSA standard (Pierce) using the BCA assay (Pierce).

Results: Pfu mutants V93E and V93R were purified to 90% purity as determined by SDS-PAGE.

### Example 5

Determining Pfu Mutant Polymerase Unit Concentration and Specific Activity

The unit concentration of purified Pfu mutant preparations was determined by PCR. In this assay, a 500 bp lacZ target is amplified from transgenic mouse genomic DNA using the forward primer: 5′-GACAGTCACTCCGGCCCG-3′ and the reverse primer: 5′ -CGACGACTCGTGGAGCCC-3′. Amplification reactions consisted of 1× cloned Pfu PCR buffer, 100 ng genomic DNA, 150 ng each primer, 200 μM each dNTP, and varying amounts of either wild type Pfu (1.25 U to 5 U) or Pfu mutant (0.625-12.5 U). Amplification was performed using a RoboCycler® temperature cycler (Stratagene) with the following program: (1 cycle) 95° C. for 2 minute; (30 cycles) 95° C. for 1 minute, 58° C. for 1 minute, 72° C. for 1.5 minutes; (1 cycle) 72° C. for 7 minutes. PCR products were examined on 1% agarose gels containing ethidium bromide.

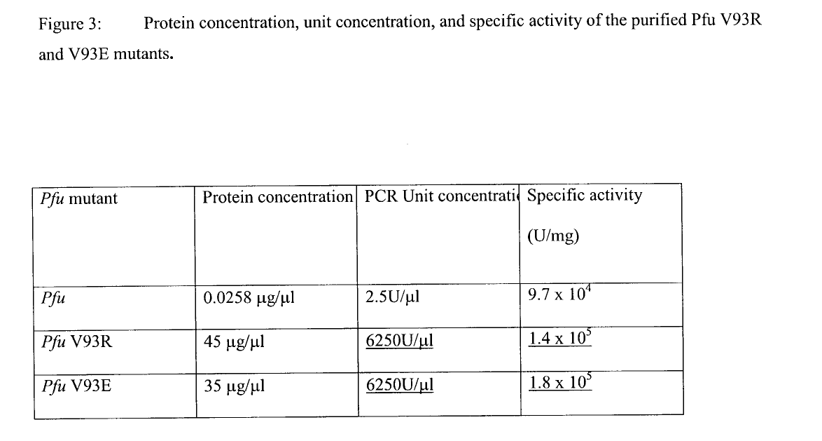

Results: FIG. 3 contains a table listing the protein concentration, unit concentration, and specific activity of the purified Pfu V93R and V93E mutants.

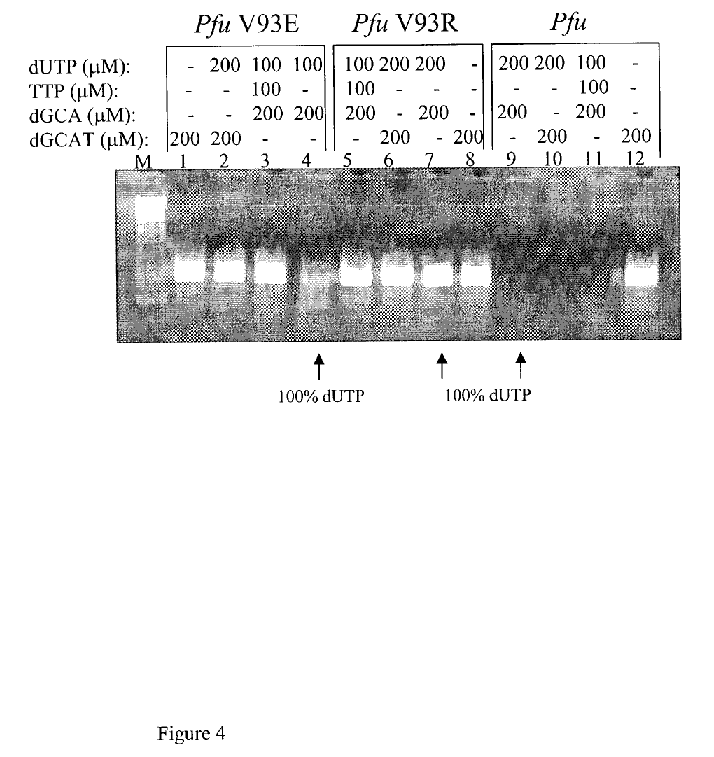

The purified mutants were also re-assayed to assess dUTP incorporation during PCR, according to the method described in Example 3. FIG. 4 shows that the Pfu V93R mutant produces similar yields of the 500 bp amplicon in the presence of 100% TTP (lane 8), 50% TTP:50% dUTP (lane 5), and 100% dUTP (lane 7), while the Pfu V93E mutant produces high yields in the presence of 100% TTP (lane 1) and 50% TTP:50% dUTP (lane 3) and lower yields in the presence of 100% dUTP (lane 4). In contrast, cloned Pfu can only amplify in the presence of 100% TTP (lane 12). These results indicate that the V93R and V93E mutations significantly improve dUTP incorporation compared to wild type Pfu, and that the V93R mutation appear to be superior to the V93E mutation with respect to reducing uracil detection.

### Example 6

PCR Amplification with Purified Pfu Mutants

PCR reactions are conducted under standard conditions in cloned Pfu PCR buffer (10 mM KCl, 10 mM (NH₄)₂SO₄, 20 mM Tris HCl (pH 8.8), 2 mM Mg SO₄, 0.1% Triton X-100, and 100 μg/ml BSA) with various amounts of cloned Pfu, PfuTurbo, or mutant Pfu DNA polymerase. For genomic targets 0.3-9 kb in length, PCR reactions contained 100 ng of human genomic DNA, 200 μM each dNTP, and 100 ng of each primer. For genomic targets >9 kb in length, PCR reactions contained 250 ng of human genomic DNA, 500 μM each dNTP, and 200 ng of each primer.

Cycling Conditions:

| Target | | |
|---|---|---|
| size | | |
| (kb) | Target gene | Cycling Parameters |
| 0.5 | LacZ | RoboCycler |
| | (transgenic mouse | (1 cycle) 95° C. 2 min |
| | genomic DNA) | (30 cycles) 95° C. 1 min, 58° C. 1 min, |
| | | 72° C. 1.5 min |
| | | (1 cycle) 72° C. 7 min |
| 2.3 | Pfu pol | RoboCycler |
| | (5 ng plasmid | (1 cycle) 95° C. 1 min |
| | DNA) | (30 cycles) 95° C. 1 min, 56° C. 1 min, |
| | | 72° C. 4 min |
| | | (1 cycle) 72° C. 10 min |
| 12 | Hα1AT | Perkin/Elmer 9600 |
| | | (1 cycle) 92° C. 2 min |
| | | (10 cycles) 92° C. 10 sec, 58° C. 30 sec, |
| | | 68° C. 18 min |
| | | (20 cycles) 92° C. 10 sec, 58° C. 30 sec, |
| | | 68° C. 24 min |
| | | (1 cycle) 68° C. 10 min |

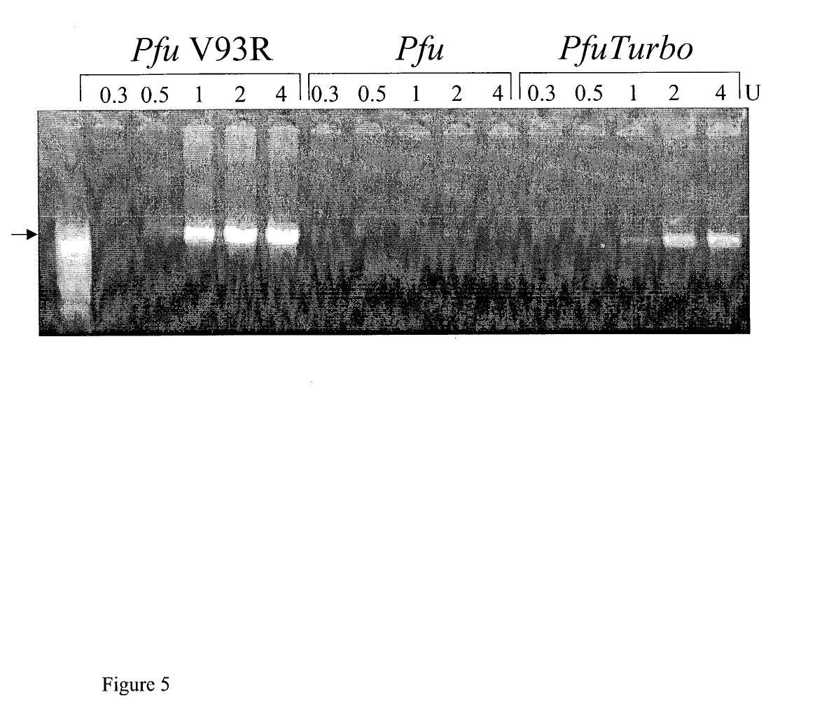

Results. Comparisons were carried out to determine if mutations that improve dUTP incorporation, and hence reduce uracil detection, also improve PCR performance. In FIG. 5, a 12 kb target was amplified from human genomic DNA using 2 min per kb extension times. Under these conditions, 1 U, 2 U, and 4 U of the Pfu V93R mutant successfully amplified the target, while the same amount of cloned Pfu could not. In comparison, PfuTurbo successfully amplified the long target; however, PCR product yields were significantly lower than those produced with the V93R mutant (FIG. 5). Similar experiments employing 1 min per kb extension times showed that the 12 kb target could be amplified in high yield with 5 U and 10 U of Pfu V93R and amplified in low yield with 10 U of PfuTurbo (data not shown). In total, these results demonstrate that the V93R mutation dramatically improves the PCR performance of Pfu DNA polymerase.

Similar testing of the purified Pfu V93E mutant showed that although the V93E mutation improves dUTP incorporation (FIG. 2), this mutant is not robust enough to amplify the long 12 kb amplicon when assayed using enzyme amounts between 0.6 U and 10 U (data not shown). In comparison, the product was successfully amplified using 10 U of PfuTurbo (data not shown).

All patents, patent applications, and published references cited herein are hereby incorporated by reference in their entirety. While this invention has been particularly shown and described with references to preferred embodiments thereof, it will be understood by those skilled in the art that various changes in form and details may be made therein without departing from the scope of the invention encompassed by the appended claims.

What is claimed is:

1. A mutant archaeal DNA polymerase with a reduced base analog detection activity, wherein said mutant archaeal DNA polymerase comprises a mutation at position V93, wherein said mutation is a Valine to Arginine substitution, a Valine to Glutamic acid substitution, a Valine to Lysine substitution, a Valine to Aspartic acid substitution or a Valine to Asparagine substitution.

2. A mutant Pfu DNA polymerase with a reduced base analog detection activity, wherein said mutant Pfu DNA polymerase comprises a Valine to Arginine substitution or a Valine to Glutamic acid substitution or a Valine to Lysine substitution or a Valine to Aspartic acid substitution or a Valine to Asparagine substitution at amino acid position V93.

3. The mutant DNA polymerases of claim 1 or 2, wherein said mutant DNA polymerase further comprises a Glycine to Proline substitution at amino acid position 387 (G387P) that confers a reduced DNA polymerization phenotype to said mutant Pfu DNA polymerases.

4. The mutant DNA polymerases of claim 1 or 2, wherein said mutant DNA polymerase further comprises an Aspartate to alanine substitution at amino acid 141 (D141 A) and a Glutamic acid to Alanine substitution at amino acid position 143 (D141A/E143A) that renders said mutant DNA polymerase 3′-5′ exonuclease deficient.

5. The mutant DNA polymerases of claim 1 or 2, wherein said mutant DNA polymerase is a chimera that further comprises a polypeptide that increases processivity and/or salt resistance.

6. A mutant archael DNA polymerase with a reduced base analog detection activity comprising a deletion or an insertion.

7. An isolated polynucleotide comprising a nucleotide sequence encoding a mutant archacal DNA polymerase having a reduced base analog detection activity, wherein said mutant archaeal DNA polymerase comprises a mutation at position V93, wherein said mutation is a Valine to Arginine substitution, a Valine to Glutamic acid substitution, a Valine to Lysine substitution, a Valine to Aspartic acid substitution or a Valine to Asparagine substitution.

8. An isolated polynucleotide comprising a nucleotide sequence encoding a mutant Pfu DNA polymerase having a reduced base analog detection activity, wherein said mutant Pfu DNA polymerase comprises a Valine to Arginine substitution or a Valine to Glutamic acid substitution or Valine to Lysine substitution or Valine to Aspartic acid substitution or Valine to Asparagine substitution at amino acid position V93.

9. The isolated polynucleotide of claim 7 or 8, wherein said nucleotide sequence further comprises a Glycine to Proline substitution at amino acid position 387 (G387P) that confers a reduced DNA polymerization phenotype to said mutant Pfu DNA polymerases.

10. The isolated polynucleotide of claim 7 or 8, further comprising a nucleotide sequence encoding an Aspartate to alanine substitution at amino acid 141 (D141A) and a Glutamic acid to Alanine substitution at amino acid position 143 (E143A) that confers a 3′-5′ exonuclease deficient phenotype to said mutant Pfu DNA polymerases.

11. The isolated polynucleotide of claims 7 or 8, further comprising a nucleotide sequence encoding a chimera that further encodes a polypeptide that increases processivity and/or salt resistance.

12. An isolated polynucleotide comprising a nucleotide sequence encoding a mutant archael DNA polymerase comprising an insertion or a deletion.

13. A composition comprising a mutant archaeal DNA polymerase having a reduced base analog detection activity, wherein said mutant archaeal DNA polymerase comprises a mutation at position V93, wherein said mutation is a Valine to Arginine substitution or a Valine to Glutamic acid substitution or Valine to Lysine substitution or Valine to Aspartic acid substitution or Valine to Asparagine substitution.

14. A composition comprising a mutant Pfu DNA polymerase having a reduced base analog detection activity, wherein said mutant Pfu DNA polymerase comprises a Valine to Arginine substitution or a Valine to Glutamic acid substitution or Valine to Lysine substitution or Valine to Aspartic acid substitution or Valine to Asparagine substitution at amino acid position V93.

15. A composition comprising a mutant archeal DNA polymerase having a reduced base analog detection activity wherein said mutant DNA polymerase is a chimera that comprises a polypeptide that increases processivity and/or salt resistance.

16. A composition comprising a mutant archael DNA polymerase having reduced base analog detection activity wherein said mutant DNA polymerase comprises an insertion or a deletion.

17. The composition of claim 13 or 14, further comprising Taq DNA polymerase.

18. The composition of claim 17, wherein said Taq DNA polymerase is at a 2 fold, 5 fold, 10 fold or 100 fold lower concentration than said mutant Pfu DNA polymerase.

19. The composition of claim 13, 14, 15, or 16, further comprising a PCR enhancing factor and/or an additive.

20. The composition of claim 13 or 14, further comprising a PfuG387P DNA polymerase or a Pfu G387P/V93R or G387P/V93E or G387P/V93 K, G387P/V93 D, or G387P/V93N double mutant DNA polymerase.

21. The composition of claim 13 or 14 further comprising Taq and a mutant archael DNA polymerase selected from the group consisting of G387P/V93R, G387P/V93D, G387P/V93E, G387P/V93K and G387P/V93N.

22. The composition of claim 21, further comprising PEF.

23. The composition of claim 20, further comprising a PCR enhancing factor and/or an additive.

24. The composition of claim 14, further comprising a Pfu V93R/D141A/E143A, a V93E/D141A/E143A, a V93 K/D141A/E143A, a V93 D/D141A/E143A, or a V93N/D141A/E143A triple mutant.

25. The composition of claim 24, further comprising a PCR enhancing factor and/or an additive.

26. The composition of claim 14, further comprising a Pfu V93R/D141A/E143A, a V93E/D141A/E143A, a V93 K/D141A/E143A, a V93 D/D141A/E143A, or a V93N/D141A/E143A triple mutant

27. The composition of claims 13 or 14, further comprising a Thermus DNA ligase or a FEN-1 nuclease.

28. The composition of claim 27, further comprising a PCR enhancing factor and/or an additive.

29. A kit comprising a mutant archacal DNA polymerase having a reduced base analog detection activity, wherein said mutant archaeal DNA polymerase comprises a mutation at position V93, wherein said mutation is a Valine to Arginine substitution or a Valine to Glutamic acid substitution or Valine to Lysine substitution or Valine to Aspartic acid substitution or Valine to Asparagine substitution, and packaging materials therefor.

30. A kit comprising a mutant Pfu DNA polymerase having a reduced base analog detection activity, wherein said mutant Pfu DNA polymerase comprises a Valine to Arginine substitution or a Valine to Glutamic acid substitution or Valine to Lysine substitution or Valine to Aspartic acid substitution or Valine to Asparagine substitution at amino acid position V93.

31. The kit of claim 29 or 30, further comprising a PCR enhancing factor and/or an additive.

32. The kit of claim 29 or 30, further comprising Taq DNA polymerase.

33. The kit of claim 32, wherein said Taq DNA polymerase is at a 2 fold, 5 fold, 10 fold or 100 fold lower concentration than said mutant Pfu DNA polymerase.

34. The kit of claim 32, further comprising a PCR enhancing factor and/or an additive.

35. The kit of claim 29 or 30, further comprising a Pfu G387 single mutant or a Pfu G387P/V93R or G387P/V93 E or G387P/V93 K or G387P/V93 D or G387P/V93N double mutant DNA polymerase.

36. The kit of claim 35, further comprising a PCR enhancing factor and/or an additive.

37. The kit of claim 30, wherein said mutant Pfu DNA polymerase further comprises a D141A/E143A mutation.

38. The kit of claim 37, further comprising a PCR enhancing factor and/or an additive.

39. The kit of claim 29 or 30, further comprising Thermus DNA ligase, FEN-1 nuclease or a PCR enhancing factor and/or an additive and packaging materials therefor.

40. A kit comprising a mutant archael DNA polymerase with a reduced base analog detection activity wherein said mutant DNA polymerase is a chimera that comprises a polypeptide that increases processivity and/or salt resistance.

41. The kit of claim 40, further comprising a PCR enhancing factor and/or an additive.

42. The kit of claim 40, further comprising Thermus DNA ligase, FEN-1 nuclease or a PCR enhancing factor and/or an additive and packaging materials therefor.

43. A method for DNA synthesis comprising:
(a) providing a mutant archaeal DNA polymerase having a reduced base analog detection activity, wherein said mutant archaeal DNA polymerase comprises a mutation at position V93, wherein said mutation is a Valine to Arginine substitution or a Valine to Glutamic acid substitution or Valine to Lysine substitution or Valine to Aspartic acid substitution or Valine to Asparagine substitution; and contacting said enzyme with a nucleic acid template, wherein said enzyme permits DNA synthesis.

44. A method for DNA synthesis comprising:
(a) providing a mutant Pfu DNA polymerase having a reduced base analog detection activity, wherein said mutant Pfu DNA polymerase comprises a Valine to Arginine substitution or a Valine to Glutamic acid substitution or Valine to Lysine substitution or Valine to Aspartic acid substitution or Valine to Asparagine substitution at amino acid position V93; and
(b) contacting said enzyme with a nucleic acid template, wherein said enzyme permits DNA synthesis.

45. The method of claim 43 or 44, wherein said DNA synthesis is performed in the presence of dUTP.

46. A method for cloning of a DNA synthesis product comprising:
(a) providing a mutant archaeal DNA polymerase having a reduced base analog detection activity, wherein said mutant archaeal DNA polymerase comprises a mutation at position V93, wherein said mutation is a Valine to Arginine substitution or a Valine to Glutamic acid substitution or Valine to Lysine substitution or Valine to Aspartic acid substitution or Valine to Asparagine substitution; and
(b) contacting said mutant archaeal DNA polymerase with a nucleic acid template, wherein said mutant archaeal DNA polymerase permits DNA synthesis to generate a synthesized DNA product; and
(c) inserting said synthesized DNA product into a cloning vector.

47. A method for cloning of a DNA synthesis product comprising:
(a) providing a mutant Pfu DNA polymerase having a reduced base analog detection activity, wherein said mutant Pfu DNA polymerase comprises a Valine to Arginine substitution or a Valine to Glutamic acid substitution or Valine to Lysine substitution or Valine to Aspartic acid substitution or Valine to Asparagine substitution at amino acid position V93;
(b) contacting said mutant Pfu DNA polymerase with a nucleic acid template, wherein said mutant Pfu DNA polymerase permits DNA synthesis to generate a synthesized DNA product; and
(c) inserting said synthesized DNA product into a cloning vector.

48. The method of claim 43, 44, 46 or 47, further comprising a Thermus DNA ligase or a FEN-1 nuclease.

49. A method for sequencing DNA comprising the step of:
(a) providing a mutant archaeal DNA polymerase having a reduced base analog detection activity, wherein said mutant archaeal DNA polymerase comprises a mutation at position V93, wherein said mutation is a Valine to Arginine substitution-or a Valine to Glutamic acid substitution or Valine to Lysine substitution or Valine to Aspartic acid substitution or Valine to Asparagine substitution;
(b) generating chain terminated fragments from the DNA template to be sequenced with said mutant archaeal DNA polymerase in the presence of at least one chain terminating agent and one or more nucleotide triphosphates, and
(c) determining the sequence of said DNA from the sizes of said fragments.

50. A method for sequencing DNA comprising the step of:
(a) providing a mutant Pfu DNA polymerase having a reduced base analog detection activity, wherein said mutant Pfu DNA polymerase comprises a Valine to Arginine substitution or a Valine to Glutamic acid substitution or Valine to Lysine substitution or Valine to Aspartic acid substitution or Valine to Asparagine substitution at amino acid position V93;
(b) generating chain terminated fragments from the DNA template to be sequenced with said mutant Pfu DNA polymerase in the presence of at least one chain terminating agent and one or more nucleotide triphosphates, and
(c) determining the sequence of said DNA from the sizes of said fragments.

51. The method of claim 43, 44, 46, 47, 49 or 50, further providing Taq DNA polymerase.

52. The method of claim 44, wherein said Taq DNA polymerase is at a 2 fold, 5 fold, 10 fold or 100 fold lower concentration than said mutant Pfu DNA polymerase.

53. The method of claim 43, 44, 46, 47, 49 or 50, further comprising a PCR enhancing factor and/or an additive.

54. The method of claim 43, 44, 46 or 47, further providing a Pfu G387P single mutant, a Pfu G387P/V93R or G387P/V93 E or G387P/V93 K or G387P/V93 D or G387P/V93N double mutant DNA polymerase or an archeal DNA polymerase mutant that is a chimera comprising a polypeptide that increases processivity and/or salt resistance.

55. The method of claim 54, further comprising a PCR enhancing factor and/or an additive.

56. The method of claim 43, 44, 46, 47, 49 or 50, further providing a Pfu D141A/E143A double mutant DNA polymerase.

57. The method of claim 56, further comprising a PCR enhancing factor and/or an additive.
